(19) United States
(12) Patent Application Publication
(10) Pub. No.: US 2023/0355763 A1
(43) Pub. Date: Nov. 9, 2023

(54) NANOPARTICLE VACCINES AND USES THEREOF FOR PROPHYLAXIS AND TREATMENT OF ATHEROSCLEROSIS USING PEPTIDE 210 AS AN ANTIGEN

(71) Applicants: CEDARS-SINAI MEDICAL CENTER, Los Angeles, CA (US); University of Southern California, Los Angeles, CA (US)

(72) Inventors: Prediman K. Shah, Los Angeles, CA (US); Kuang-Yuh Chyu, Los Angeles, CA (US); Eun Ji Chung, Rancho Palos Verdes, CA (US); Noah Trac, Covina, CA (US)

(73) Assignees: CEDARS-SINAI MEDICAL CENTER, Los Angeles, CA (US); University of Southern California, Los Angeles, CA (US)

(21) Appl. No.: 18/312,194

(22) Filed: May 4, 2023

Related U.S. Application Data
(60) Provisional application No. 63/338,321, filed on May 4, 2022.

(51) Int. Cl. A61K 39/00; A61P 37/04; B82Y 5/00; A61K 39/385

(52) U.S. Cl. CPC A61K 39/4634; A61P 37/04; B82Y 5/00; A61K 39/385

(57) ABSTRACT

Described herein are nanoparticle bound self-antigens as an immune and vaccine formulations to elicit self-regulations and reduce atherosclerosis. The use of apolipoprotein B100 (ApoB-100) peptide P210 was investigated in self-assembling peptide amphiphile micelles (P210-PAM) as a vaccine formulation to reduce atherosclerosis in ApoE^{-/-} mice. Demonstrated herein, P210 provided T cell activation and memory response in peripheral blood mononuclear cells of human subjects with atherosclerotic cardiovascular disease, and dendritic cell uptake of P210-PAM and its co-staining with major histocompatibility complex class I (MHC-I) molecules supported its use as an immunogenic composition. In ApoE^{-/-} mice, immunization with P210-PAM dampened P210-specific CD4⁺ T cell proliferative response and CD8⁺ T cell cytolytic response, modulated macrophage phenotype, and significantly reduced aortic atherosclerosis. P210-PAM immunization also reduced atherosclerosis in chimeric mice with human MHC-I allele.

### CROSS-REFERENCE TO RELATED APPLICATIONS

This application includes a claim of priority under 35 U.S.C. § 119(e) to U.S. provisional patent application No. 63/338,321, filed May 4, 2022, the entirety of which is hereby incorporated by reference.

### STATEMENT REGARDING FEDERALLY SPONSORED RESEARCH OR DEVELOPMENT

This invention was made with Government support under grant nos. HL124279 and DK121328 awarded by the National Institutes of Health. The Government has certain rights in the invention.

### REFERENCE TO SEQUENCE LISTING

This application contains a Sequence Listing submitted as an electronic file named “065472_000894USPT_SequenceListing.xml”, having a size in bytes of 42,205 bytes, and created on May 3, 2023 (production date noted as 2023-05-04). The information contained in this electronic file is hereby incorporated by reference in its entirety.

### FIELD OF INVENTION

This invention relates to atherogenic peptide-presenting nano-structures for use as vaccines or therapeutic treatment against ischemic cardiovascular diseases including atherosclerosis.

### BACKGROUND

Adaptive immune responses against self-antigens such as low-density lipoprotein (LDL), apolipoprotein B100 (ApoB-100) or certain ApoB-100 related peptide epitopes is a hallmark of experimental and human atherosclerosis. Within these adaptive immune responses, antigen specificity against one of the ApoB-100 peptides, P210, is present and plays a crucial role in atherosclerosis. In hypercholesterolemic mice, splenic CD8+ T cells specifically reactive to P210 peptide fragments can be detected using peptide-loaded synthetic soluble MHC-I pentamers (Dimayuga, P. C. et al., J. Am. Heart Assoc. 6:doi: 10.1161/JAHA.116.005318). These P210-specific CD8+ T cells increased in response to atherogenic diet, correlated with the extent of atherosclerosis, and localized to atherosclerotic plaques. In humans, P210 fragments and P210-specific antibodies have been detected in plaques and circulation of patients with atherosclerotic cardiovascular disease (ASCVD) (Per Sjogren et al., European Heart Journal (2008) 29, 2218-2226).

The effective use of peptide antigens for immunization strategies depends on the formulation. In preclinical studies, immunogenic peptides are often conjugated as haptens to carrier molecules along with an adjuvant such as mineral salt to provoke an immune response to establish vaccine efficacy. However, such formulations have their limitations in clinical translation. For example, aluminum adjuvants have limitations including local reactions, augmentation of IgE antibody responses, ineffectiveness for some antigens, and inability to augment cell-mediated immune responses, especially cytotoxic T-cell responses. Traditional aluminum salt based vaccines are known to induce weak cell-mediated immune responses, limiting their clinical application and choice of antigens. Vaccines against infections are formulated to trigger immune responses against the infectious agent and induce immunologic memory for future infectious challenges. On the other hand, vaccines targeting non-infectious inflammatory conditions induced by self-antigens are formulated towards induction of self-regulation.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

A specific class of peptide-amphiphile complex is provided, which are amphiphilic molecules each containing a peptide portion and a lipophilic portion, wherein the peptide portion is covalently bonded, complexed, or otherwise bonded to the lipophilic portion.

In various embodiments, the peptide portion contains ApoB-100 peptide 210 (P210), KTTKQSFDLSVKAQYKKNKH (SEQ ID NO:1), in a peptidyl form covalently bonded to a lipophilic molecule, such as a lipid moiety. The P210 is a hydrophilic peptide, due to the presence of multiple lysine residues, and hence a complex comprising the P210 and a lipid moiety or a lipophilic molecule forms an amphiphilic molecule. The P210 is capable of binding a human leukocyte antigen (HLA). In some embodiments, the peptide portion is P210, in a peptidyl form covalently bonded to a lipid moiety or another lipophilic molecule. In further embodiments, the peptide portion contains a fragment of P210, which is covalently or otherwise bonded to a lipid moiety or another lipophilic molecule. In yet another embodiment, the peptide portion is a fragment of P210, and the fragment is capable of binding an HLA.

In various embodiments, the lipophilic portion of the peptide-amphiphile complex includes one or two, or more hydrocarbyl groups, e.g., C₆-C₂₀ hydrocarbyl groups. In some embodiments, the lipophilic portion of the peptide-amphiphile complex includes one, two, or more alkyl chains. In some embodiments, the lipophilic portion includes two or more linear alkyl chains. In some embodiments, the lipophilic portion includes two or more alkyl chains each having 6 to 20 carbon atoms, or C₆-C₂₀ alkyl chains. In some embodiments, the lipophilic portion includes two or more linear alkyl chains each having 6 to 20 carbon atoms.

In some embodiments, a peptide-amphiphile complex having the following structure is provided:

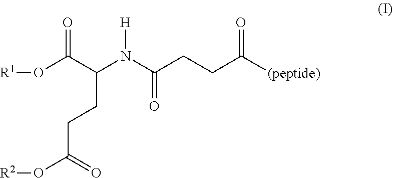

wherein:
(a) R¹ and R² are each independently C₆-C₂₀ (can be any integer between 6 and 20) hydrocarbyl groups; and
(b) the (peptide) refers to a sequence of KTTKQSFDLSVKAQYKKNKH (SEQ ID NO:1) or a fragment of SEQ ID NO:1 capable of binding a human leukocyte antigen (HLA).

In some embodiments, a peptide-amphiphile complex has a structure (II):

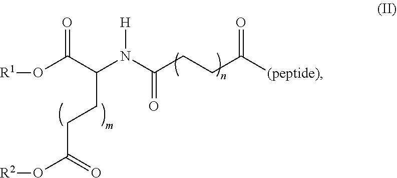

or a variant of (II), wherein the variant has any one or more of —O— or ═O in (II) BE independently substituted with S or another atom;
and wherein R¹ and R² are each independently C₆-C₂₀ (can be any integer between 6 and 20) substituted or unsubstituted hydrocarbyl groups; m and n are independently an integer (e.g., from 0 to 20) representing the number of repeats of unsubstituted or substituted —CH₂—CH₂—; and the (peptide) refers to a sequence of KTTKQSFDLSVKAQYKKNKH (SEQ ID NO:1) or a fragment of SEQ ID NO:1 capable of binding a human leukocyte antigen (HLA).

In some embodiments, R¹ and R² are independently C₁₂-C₁₆, C₈-C₁₂, C₁₆-C₂₀, C₂₀-C₃₀ substituted or unsubstituted hydrocarbyl groups. In some embodiments, R¹ and R² are independently C₆, C₇, C₈, C₉, C₁₀, C₁₁, C₁₂, C₁₃, C₁₄, C₁₅, C₁₆, C₁₇, C₁₈, C₁₉, or C₂₀ substituted or unsubstituted, alkyl or heteroalkyl groups. In some embodiments, m and n are independently selected from 1, 2, 3, 4, 5, 6, and 0.

In various embodiments, the peptide-amphiphile complex is in the form of micelles or vesicles in an aqueous medium, preferably presenting the peptide portion at the surface of the micelles or vesicles, hence forming P210-presenting (or HLA-binding) nanoparticles. In some embodiments, the micelles are in the form of nanofibers. In various embodiments, a pharmaceutical composition is provided, which includes an excipient and nanoparticles or micelles comprising a quantity of the peptide-amphiphile complex. In some embodiments, the pharmaceutical composition further includes one or more of an adjuvant, a filler, and/or a detectable label. In some embodiments, detectable label is covalently bonded to the peptide-amphiphile complex. In some embodiments, the pharmaceutical composition doesn't include a MHC molecule such as an HLA; in some embodiments, the peptide-amphiphile complex doesn't include a MHC molecule such as an HLA.

Immunogenic compositions are also provided for eliciting an immune response in a mammal (e.g., human) having an ischemic cardiovascular disease, wherein the immunogenic compositions include or are the pharmaceutical compositions disclosed herein. In some embodiments, the immunogenic compositions elicit an athero-protective effect (e.g., the generation of anti-P210 antibody) in a subject receiving the immunogenic compositions. In some embodiments, the immunogenic composition elicit a therapeutic treatment in a subject receiving the immunogenic compositions.

Methods for eliciting an immune response are provided, which includes administering an immunogenically effective amount of a peptide-amphiphile complex or a pharmaceutical composition thereof to a subject who does not have an acute coronary syndrome or a cardiovascular disease, but who may be suspected or at risk of developing atherosclerosis or an ischemic cardiovascular disease. In some embodiments, the methods are for eliciting an immune response in a subject who has had atherosclerosis or an ischemic cardiovascular disease, so as to reduce the likelihood of recurrence of atherosclerosis or the ischemic cardiovascular disease.

Methods of treating a subject with atherosclerosis or an ischemic cardiovascular disease are also provided, which includes administering to the subject a therapeutically effective amount of a peptide-amphiphile complex or a pharmaceutical composition thereof. In some embodiments, the amount is effective for reducing the amount of plaques in the cardiac blood vessels (or cardiovasculature). In some embodiments, the amount is effective for reducing cytolytic activity of CD8⁺ T cell, reducing proliferative activity of CD4⁺ T cell, reducing aortic atherosclerosis, or a combination thereof,

In some embodiments, the immunogenic composition is administered in one dose. In some embodiments, the immunogenic composition is administered in a series of doses to a subject.

Further embodiments provide methods for preparing a composition including micelles formed with a peptide-amphiphile complex disclosed herein. These methods include the steps of dissolving the peptide-amphiphile complex in an organic solvent, followed by hydrating in an aqueous medium at an increased temperature to obtain hydrated lipid suspension, and performing sonication, extrusion or another micronization technique to obtain the micellar composition composed of the peptide-amphiphile complex.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1K depict intrinsic T cell response to P210 peptide in human PBMCs. Human peripheral blood mononuclear cells from control subjects or from acute coronary syndrome (ACS) patients were cultured for 16 hours for the activation-induced marker (AIM) assay with no stimulation or stimulated with P210 peptide or CMV pooled peptides. (1A-1D) Activation state of PBMCs without peptide stimulation from control subjects or from ACS patients; CD4⁺CD25⁺ (FIG. 1A), CD4⁺CD69⁺ (FIG. 1B), CD8⁺CD25⁺ (FIG. 1C), CD8⁺CD69⁺ (FIG. 1D). (FIG. 1E-FIG. 1F) AIM cells in response to P210 or CMV peptide pool; CD4⁺CD69⁺CD134⁺ (FIG. 1E), CD8⁺CD69⁺CD134⁺ (FIG. 1F). (FIG. 1G-FIG. 1J) PBMCs stimulated with P210 peptide for 72 h and cells were stained for T effector markers (FIG. 1G, FIG. 1I) or T effector memory markers (FIG. 1H, FIG. 1J). (FIG. 1K) Gating scheme for T effector and memory analysis. Mann-Whitney test except for (1C & 1E), T-test. ‡P=0.07; †P=0.05; *P<0.05. (1A-1F) Control N=7-8, ACS N=12; some samples/treatments did not have detectable AIM(+) cells so ratio could not be determined. (1G-1J) Control N=14, ACS N=13.

FIG. 1L depicts stimulation of human PBMCs with 0.5×PMA/ionomycin cocktail served as positive control for the AIM assay. *P<0.05 Mann-Whitney. FIG. 1M depicts markers for other AIM(+) cells were not different compared to Controls after P210 stimulation.

FIG. 2A-2E depict P210-FITC uptake by mouse BMDCs. Confocal microscopy of BMDCs incubated with (FIG. 2A) FITC only or (FIG. 2B) P210-FITC. Same magnification in FIGS. 2A and 2B and bar=5 μm in FIG. 2B. (FIG. 2C) FITC internalization was quantified using flow cytometry of CD11c-stained cells. Cells were size gated and then gated on CD11c (FIG. 2C, top panel). CD11c+ cells were then analyzed on CD11c/FITC quadrants and the results plotted on a scatter graph indicating the mean percentage of FITC+ cells on the CD11c+ gate (FIG. 2C, bottom panel; N=8 each). (FIG. 2D) Heparin binds P210-FITC (No heparin N=12; 100 U heparin N=10). (FIG. 2E) proteoglycan inhibitor p-Nitrophenyl β-D xylopyranoside (pNP-xyl) blocks proteoglycan-mediated uptake of P210-FITC (N=5 each). *P<0.05, T test.

FIG. 3A-3M depict P210-PAM nanoparticles. (FIG. 3A) Majority of P210-PAM are between 15-25 nm in size (N=3). Transmission electron microscopy of P210-PAM at low (FIG. 3B) and high (FIG. 3C) magnification. (FIG. 3D) Light microscopy of Giemsa-stained mouse BMDCs. Fixed BMDCs stained with (FIG. 3E) CD11c PE, (FIG. 3F) MHC-I APC, (FIG. 3G) FITC P210-PAM and (FIG. 3H) DAPI. (FIG. 3I) Color overlay and arrows indicating costaining. The last lysine of the P210 peptide was FITC labeled prior to PAM assembly. Experiment was replicated with similar results. (FIG. 3J) CD4⁺ central memory (CM) T cells, (FIG. 3K) CD4⁺ effector memory (EM) T cells, (FIG. 3L) CD8⁺ CM T cells and (FIG. 3M) CD8⁺ EM T cells from spleens of 25 weeks old ApoE^{-/-} mice fed high fat diet for 16 weeks. Splenocytes were collected after 48 h treatment with 20 μg/ml MSA-PAM or P210-PAM. N=5 each, P<0.05 by T test.

FIG. 4A-4F depict PAM imaging and retention in vivo. In vivo imaging of P210-PAM (FIG. 4A, FIG. 4C) or MSA-PAM (FIG. 4D, FIG. 4F) retention at injection site of C57BL/6J mice over 168 h. Percent of signal intensity relative to time zero (immediately after injection) of P210-PAM (FIG. 4B) or MSA-PAM (FIG. 4E). N=4 each. Colocalization of fluorescently labelled P210-PAM (4C) or MSA-PAM (4F) with F4/80+ macrophages and CD11c+ dendritic cells at the injection site at 48 hours.

FIG. 5A-5P depict P210-PAM immunization in ApoE^{-/-} mice. (FIG. 5A-FIG. 5C) Immune regulatory profile of CD4⁺ T cells by (FIG. 5A) PD-1⁺, (FIG. 5B) CTLA4⁺, (FIG. 5C) Foxp3⁺, and (FIG. 5D & FIG. 5E) of CD8⁺ T cells by (FIG. 5D) PD-1⁺, (FIG. 5E) CTLA4⁺, in splenocytes of immunized mice 1 week after second booster. (FIG. 5F & FIG. 5G) Splenic (FIG. 5F) CD4⁺ T cell and (FIG. 5G) CD8⁺ T cell proliferation of immunized mice in response to P210 peptide or (FIG. 5H & FIG. 5I) Con A stimulation assessed by BrdU staining for (FIG. 511) CD4⁺ T cell and (FIG. 5I) CD8⁺ T cell. (FIG. 5J) CD107a to assess CD8⁺ T cell cytolytic activity in splenocytes of immunized mice. (FIG. 5K) Representative photographs of aortic en face staining with Oil red-O at 25 weeks of age. (FIG. 5L) Atherosclerosis measured as percentage of whole aorta stained by Oil red-O. Splenic mRNA expression of (FIG. 5M) IL-1β, (FIG. 5N) IL-1R1, (FIG. 5O) IL-6 and (FIG. 5P) IL-17a. Number of mice used in each group is represented by the number of dots in individual figure. *P<0.05; †P=0.05, T test except for (L) ANOVA with Holm-Sidak multiple comparisons test.

FIG. 6A-6K depict macrophage phenotype in P210-PAM immunized ApoE^{-/-} mice. Splenic IL-1R1 expression measured by MFI in (FIG. 6A) F4/80⁺ monocyte/macrophage cells, (FIG. 6B) CD4⁺ T cells, (FIG. 6C) CD8⁺ T cells, and (FIG. 6D) dendritic cells. Macrophages isolated from peritoneal cavity of immunized mice elicited by thioglycolate injection assessed for (FIG. 6E) iNOS and (FIG. 6F) arginase 1 mRNA expression. (FIG. 6G) Ratio of arginase 1 to iNOS mRNA expression. Macrophages were further phenotyped using mRNA expression of (FIG. 6H) IL-6, (FIG. 6I) MCP-1, (FIG. 6J) IL-12, and (FIG. 6K) IL-10. Number of mice used in each group is represented by the number of dots in individual figure. *P<0.05 Mann-Whitney for all figures except for (FIG. 6J) and (FIG. 6K) which were analyzed by T test.

FIG. 7A-7I depict ApOB_{KTTKQSFDL} (SEQ ID NO:2) Pentamer. (FIG. 7A) Binding scores of P210 epitope sequences listed in Table 2 from REVEAL binding assay. Representative plot of PBMCs from HLA-A*02:01(+) volunteer stained with (FIG. 7B) HLA-A*02:01 control pentamer or (FIG. 7C) ApOB_{KTTKQSFDL} (SEQ ID NO:2) pentamer, (FIG. 7D) with backgating in magenta. (FIG. 7E) ApOB_{KTTKQSFDL} (SEQ ID NO:2) pentamer⁺CD8⁺ T cells in PBMCs of HLA-A*02:01⁺ volunteers compared to control HLA-A*02:01 pentamer (N=10). (FIG. 7F) Aliquots available from 8 of the same volunteers were stimulated with 20 μg/ml P210 peptide or vehicle (sterile ddH₂O) for 5 days. Representative scatter plot of vehicle (FIG. 7G) or P210 peptide (FIG. 7H) sample stained with ApOB_{KTTKQSFDL} (SEQ ID NO:2) pentamer. (FIG. 7I) The P210 stimulated samples were also stained with HLA-A*02:01 control pentamer as reference for pentamer specificity. *P<0.05 by T test.

FIG. 8A-8O depict HLA-A*02:01 transgenic mouse model. (FIG. 8A) Functional test of transgene in A2Kb Tg ApoE^{-/-} mice immunized with A2V7 and the detection of A2V7 Pentamer⁺CD8⁺ T cells. (FIG. 8B) Representative scatter plot of A2V7 pentamer⁺CD8⁺ T cells in adjuvant or A2V7 immunized mice with backgating in magenta. (FIG. 8C) Representative photographs of Oil red-O stained en face aortas from female and male A2Kb Tg ApoE^{-/-} mice fed normal chow (NC) or high cholesterol diet (HC) for 8 or 16 weeks starting at 9 weeks of age. (FIG. 8D) Aortic atherosclerosis in female and (FIG. 8E) male mice at 17 and 25 weeks of age. (FIG. 8F-FIG. 8I) CD4⁺ Memory T cells and (FIG. 8J-FIG. 8M) CD8⁺ Memory T cells in A2Kb Tg ApoE^{-/-} mice. (FIG. 8N) HLA-A*02:01-P210 pentamer⁺CD8⁺ T cells in splenocytes of 17-week-old A2Kb Tg ApoE^{-/-} mice and (FIG. 8O) in plaques of mice aged >63 weeks old after 4 weeks of HC diet feeding; N=4. T test for 2 group comparison; ANOVA with Holm-Sidak multiple comparisons test for more than 2 groups. Number of mice in each group is represented by the number of dots in individual figure. *P<0.05; †P=0.06.

FIG. 9A-9E depicts P210-PAM immunized A2Kb Tg ApoE^{-/-} mice. (FIG. 9A) Detection of ApOB_{KTTKQSFDL} (SEQ ID NO:2) Pentamer(+) cells in splenocytes of A2Kb Tg ApoE^{-/-} mice 13 weeks after second booster injection with either PBS or P210-PAM; †P=0.08, T test. (FIG. 9B) Representative photographs of aortic atherosclerosis in these mice. (FIG. 9C) Measurement of percent aortic atherosclerosis area. (FIG. 9D) Representative photographs of aortic atherosclerosis in a second cohort of mice immunized with either MSA-PAM or P210-PAM. (FIG. 9E) Percent aortic atherosclerosis area measurement. *P<0.05, T test.

FIG. 10 depicts a reaction scheme to synthesize diC₁₆, through intermediate 1′-3′-dihexadecyl L-glutamate and 1′-3′-dihexadecyl N-succinyl-L-glutamate (diC₁₆), as well as 1′-3′-dihexadecyl L-glutamate which is further deprotonated and inserted with a spacer molecule (succinic anhydride) to form the diC₁₆ tail.

FIG. 11 depicts MALDI characterization of p210 peptide (panel A, expected m/z: 3058), MSA peptide (panel B, expected m/z: 2882), and diC16-cy7 (panel C, expected m/z: 1326) amphiphiles.

FIG. 12 depicts lack of effect on DC phenotype by P210-PAM immunization. (panel A) CD11c+CD40+, (panel B) CD11c+CD80+, (panel C) CD11c+CD86+, and (panel D) CD11c+PD-L1+ DCs were not significantly different between MSA-PAM (N=4) and P210-PAM (N=5) immunized mice.

FIG. 13A-13G depict (FIG. 13A) Serum cholesterol, (FIG. 13B) serum LDL, (FIG. 13C) serum HDL, (FIG. 13D) anti-P210 IgM, and (FIG. 13E) anti-P210 IgG in immunized ApoE^{-/-} mice. Anti-P210 isotypes (FIG. 13F) IgG1 and (FIG. 13G) IgG2b isotypes were further characterized. *P<0.05 Kruskal-Wallis followed by Dunn's multiple comparisons test except for (13D); Chi-square for IgG1 to compare presence or absence of detectable anti-P210 IgG1; P<0.0001.

FIG. 14 depicts representative photos of Oil Red-O (ORO) stained aortic sinus plaques from (panel A) PBS, (panel B) MSA-PAM and (panel C) P210-PAM immunized ApoE^{-/-} mice. (panel D) Plaque size and (panel E) ORO percent stained area measurements. Representative photos of CD68 stained aortic sinus plaques from (panel F) PBS, (panel G) MSA-PAM and (panel H) P210-PAM mice. (panel I) CD68 percent stained area measurements. Bar=0.1 mm.

FIG. 15 depicts (panel A) body weight of female A2Kb Tg ApoE^{-/-} and (panel B) male A2Kb Tg ApoE^{-/-} mice at 17 weeks or 25 weeks of age fed with normal chow (NC) or high cholesterol diet (HC) for 8 or 16 weeks. (panel C) Aortic sinus plaque size in female A2Kb Tg ApoE^{-/-} mice and (panel D) male A2Kb Tg ApoE^{-/-} mice at 17 weeks or 25 weeks of age fed with normal chow (NC) or high cholesterol diet (HC) for 8 or 16 weeks. ANOVA with Holm-Sidak multiple comparisons test. *P<0.05.

FIG. 16 depicts (in panels A & C) serum total cholesterol and (in panels B & D) serum LDL levels in immunized A2Kb Tg ApoE^{-/-} mice. *P<0.05. (panels E-H) Aortas from 25 weeks old immunized A2Kb Tg ApoE^{-/-} mice of each group were subjected to enzymatic digestion and the recovered cells stained for (panel E) CD3, (panel F) CD4, (panel G) CD8, and (panel H) F4/80 for monocyte/macrophage.

FIG. 17A depicts PCR products of A2Kb chimeric gene (Lane 1: A2Kb product; Lane 2: 1 kb plus DNA ladder). FIG. 17B depicts Cloning of A2Kb chimeric gene into pCR-XL-TOPO T vector. FIG. 17C depicts A2Kb fragments obtained by digesting recombinant plasmids with Hind III, BamH I and Hinc II (Lane1: 1 kb plus DNA ladder; Lane 2: Products of digesting recombinants with Hinc II; Lane 3 & 4: Products of digesting recombinants with Hind III, BamH I and Hinc II; arrow indicates the fragments purified and used for embryo microinjection). FIG. 17D depicts HLA A*0201 fragments used for PCRs screening A2Kb transgenic ApoE^{-/-} mice. FIG. 17E depicts that chimeras carrying A2Kb gene were screened by PCR detecting HLA A*0201 fragments (Lane1: 100 bp ladder; Lane 2: 148 bp; Lane 3: 309 bp; Lane 4: 252 bp; Lane 5: 195 bp). FIG. 17F depicts flow cytometric analysis of A2Kb expression on PBMCs of A2Kb transgenic ApoE^{-/-} mice. FIG. 17G depicts that RT-PCR detecting a 1092 bp A2Kb mRNA fragment show full-length of A2Kb gene been integrated into the mice genome and efficiently transcribed (Lane 1, 2 & 4: A2Kb (+) offspring; Lane 3: A2Kb (−) offspring).

FIG. 18 is a diagram depicting the study in the Examples.

FIG. 19 depicts an experimental diagram of administering P210-PAM twice and the result showing P210-PAM induced CD4⁺ T cell response in female A2Kb-Tg ApoE^{-/-} mice.

FIG. 20 depicts, in the same experiment of FIG. 19, P210-PAM also induced CD8⁺ T cell response in female A2Kb-Tg ApoE^{-/-} mice.

FIG. 21 depicts, in the same experiment of FIG. 19, P210-PAM significantly reduced CD8⁺ T_{CM} cells in female compared to male mice in both the control and P210-PAM vaccinated mice.

FIG. 22 depicts, in the same experiment of FIG. 19, there was no sex dependent difference in CD4⁺ T_{EM} and T_{CM} cells by P210-PAM immunization.

FIG. 23 depicts an experimental diagram of administering P210-PAM three times and the results that P210-PAM significantly increased splenic CD11b^{hi} monocytes/macrophages in female compared to male mice and that P210-PAM significantly reduced Ly6C⁺CCR2⁺ monocytes in female compared to male mice.

FIG. 24 depicts, in the same experiment of FIG. 23, P210-PAM significantly reduced surface IL-1R1 expression on splenic F4/80+ macrophages in female compared to male mice as determined by MFI.

FIG. 25 depicts an experimental diagram and the results of a lack of effect of P210-PAM immunization on established atherosclerosis in mice with persistent hypercholesterolemia.

FIG. 26 depicts an experimental diagram and the results that P210-PAM immunization reduced established atherosclerosis in female mice with cholesterol lowering by diet.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Peptide amphiphile micelles (PAMs)” generally refers to a nanomaterial comprised of peptide amphiphile (PA) molecules, including a hydrophobic moiety (e.g., lipid tail) attached to a hydrophilic headgroup, which self-assemble into micelles. In some embodiments, a hydrophobic moiety is attached to an ApoB-100 peptide, such as P210, forming an amphiphilic molecule, and a plurality of these molecules assemble into a micelle. The micelles can be in a shape including but not limited to a sphere, a cylinder, an oval, or a prism. In various embodiments, the PAM has a cross-sectional size (e.g., diameter) in the nanometer range, e.g., between 1-100, 100-200, 200-300, 300-400, 400-500, 500-600, 600-700, 700-800, 800-900, or 900-1,000 nm.

“Acute coronary syndrome” (ACS) refers to a heart condition resulting from the sudden reduction of blood flow to the heart, which leads to shortness of breath and sudden chest pain. Examples of acute coronary syndrome include but are not limited to ST-elevation myocardial infarction, non-ST elevation myocardial infarction, and unstable angina. In various embodiments, subjects with ACS have atherosclerotic cardiovascular disease (ASCVD).

“Atherosclerotic cardiovascular disease” involves plaque buildup in arterial walls which includes conditions such as acute coronary syndrome and peripheral artery disease, and can cause a heart attack, stable or unstable angina, stroke, transient ischemic attack (TIA) or aortic aneurysm.

The term “treat,” or “treating” or “treatment” as used herein indicates any activity that is part of a medical care for, or that deals with, a condition medically or surgically. The term “preventing” or “prevention” as used herein indicates any activity, which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject,” “patient,” or “individual” may be used interchangeably unless otherwise noted. It refers to vertebrates such as mammals and more particularly human beings. In some embodiments, the subject has been previously identified as having an increased risk of ischemic vascular disease based on the detection of conditions typically associated with an increased risk of ischemic vascular disease (e.g., atherosclerosis). In some embodiments, the subject has not been identified as having an increased risk of ischemic vascular disease. In some embodiments, no investigation as to the risk for ischemic vascular disease or atherosclerosis in the subject has been performed.

“Lipid moiety” refers to a moiety having at least one lipid. Lipids are small molecules having hydrophobic or amphiphilic properties and are useful for preparation of vesicles, micelles and liposomes. Lipids include, but are not limited to, fats, waxes, fatty acids, cholesterol, phospholipids, monoglycerides, diglycerides and triglycerides. The fatty acids can be saturated, mono-unsaturated or poly-unsaturated. Examples of fatty acids include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26). The lipid moiety can include several fatty acid groups using branching groups such as lysine and other branched amines.

The term “hydrocarbyl” and “hydrocarbyl group” are used interchangeably. The term “hydrocarbyl group” refers to any C₁-C₂₀ (or longer) hydrocarbon group bearing at least one unfilled valence position when removed from a parent compound. Suitable “hydrocarbyl” and “hydrocarbyl groups” may be optionally substituted. The term “hydrocarbyl group having 1 to about 20 carbon atoms” refers to an optionally substituted moiety selected from a linear or branched C₁-C₂₀ alkyl, a C₃-C₂₀ cycloalkyl, a C₆-C₂₀ aryl, a C₂-C₂₀ heteroaryl, a C₁-C₂₀ alkylaryl, a C₇-C₂₀ arylalkyl, and any combinations thereof.

The term “alkyl” refers to a straight chain, branched and/or cyclic (“cycloalkyl”) hydrocarbon having from 1 to 40 (e.g., 1 to 10, 11 to 20, 21 to 30, or 30 to 40) or more carbon atoms, e.g., C₁-C₄₀ (including any integer number of carbon atoms between 1 and 40). Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

The term “alkenyl” refers to a straight chain, branched and/or cyclic hydrocarbon having from 2 to 40 (e.g., 2 to 10 or 11 to 20) or more carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

The term “alkynyl” refers to a straight chain, branched or cyclic hydrocarbon having from 2 to 40 (e.g., 2 to 20 or 21 to 40) or more carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

The term “alkoxy” refers to an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

The term “alkylaryl” or “alkyl-aryl” refers to an alkyl moiety bound to an aryl moiety. The term “arylalkyl” or “aryl-alkyl” means an aryl moiety bound to an alkyl moiety. The term “aryl” refers to an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

The term “heteroalkyl” refers to an alkyl moiety in which at least one of its carbon atoms has been replaced with a heteroatom (e.g., N, O or S).

The term “heteroaryl” refers to an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyridinium, pyrimidinyl, pyrimidyl, pyrrolyl, quinazolinyl, quinolinyl, tetrazolyl, thiazolyl, and triazinyl.

The term “alkylheteroaryl” or “alkyl-heteroaryl” refers to an alkyl moiety bound to a heteroaryl moiety. The term “heteroarylalkyl” or “heteroaryl-alkyl” means a heteroaryl moiety bound to an alkyl moiety.

The term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

The term “heterocyclealkyl” or “heterocycle-alkyl” refers to a heterocycle moiety bound to an alkyl moiety.

The term “substituted,” when used to describe a chemical structure or moiety, refers to a derivative of that structure or moiety wherein one or more of its hydrogen atoms is substituted with a chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino; quaternary tetralkylammonium), aroyl, aryl, heteroaryl, heteroarylalkyl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl3, —CF3, —C(CF3)3), heteroalkyl, hemiacetal, imine (primary and secondary), isocyanate, isothiocyanate, ketone, nitrile, nitro, oxo, phosphodiester, sulfide, sulfonamido (e.g., SO2NH2), sulfone, sulfonyl (including alkyl sulfonyl, aryl sulfonyl and arylalkylsulfonyl), sulfoxide, thiol (e.g., sulfhydryl, thioether) and urea (—NHCONH-alkyl-). Substitutions are optionally functionalized with one or more functional groups of hydroxyl, thiol, thioether, ketone, aldehyde, ester, ether, amine, imine, amide, nitro, carboxylic acid, disulfide, carbonate, isocyanate, carbodiimide, carboalkoxy, peroxo, anhydride, carbamate, and halogen.

The term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, organic salts made from lysine, N,N′-dibenzylethylenediamine, chloroprocaine, choline, diethanolamine, ethylenediamine, meglumine (N-methylglucamine) and procaine, or metallic salts made from aluminum, calcium, lithium, magnesium, potassium, sodium and zinc. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

The term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Atherosclerosis is a disease that causes a thickening of the innermost layer (the intima) of arteries. It decreases-blood flow and may cause ischemia and tissue destruction in organs supplied by the affected vessel. Atherosclerosis is the major cause of cardiovascular disease including myocardial infarction, stroke and peripheral artery disease. Without wishing to be bound by a particular theory, the disease is initiated by accumulation of lipoproteins, primarily low-density lipoprotein (LDL), in the extracellular matrix of the vessel. These LDL particles aggregate and undergo oxidative modification. Oxidized LDL is toxic, causes vascular inflammation/injury, and initiates plaque formation. Atherosclerosis represents in many respects a response to this injury including inflammation and fibrosis. Epitopes in oxidized LDL are recognized by the immune system and give rise to antibody formation.

Modulation of the adaptive immune responses against LDL, ApoB-100 or related peptides via immunization approach has consistently reduced atherosclerosis. We previously demonstrated that P210, a 20 amino acid apoB-100 related peptide, when used in an active immunization strategy, elicited CD8⁺ T cell response to reduce atherosclerosis (Kuang-Yuh Chyu et al., PLoS ONE, February 2012, volume 7, issue 2, e30780). An outcome of experimental strategies for P210 immune modulation is alteration of T cell responses to P210, indicating that the peptide or derivatives thereof are self-antigens that provoke immune responses involved in atherosclerosis. Based on these observations, we conceive that modification of immune response to P210 can be applied in reducing human atherosclerosis.

Nanoparticle based vaccine formulations have the potential to achieve the effect of inducing self-regulation via self-antigen presenting vaccines targeting non-infectious inflammatory conditions, due to the favorable physicochemical properties of nanoparticles to provide size-preferential lymphatic transport, relatively long injection-site retention and circulating time for contact with dendritic cells, acting as adjuvants in subunit vaccines, and the induction of auto-immunity specific regulatory immune responses.

Herein we utilize a peptide amphiphile (PA) nanoparticle platform in which peptide headgroups are chemically conjugated to hydrophobic tails resulting in structures with hydrophobic and hydrophilic regions, facilitating subsequent self-assembly into well-defined peptide amphiphile micelles (PAMs). PAMs are comprised of biocompatible lipids and peptides and are chemically versatile, allowing for the incorporation of multiple modalities such as fluorescence and immunogenicity into a single particle. We have demonstrated this PAM-based platform can be a new immunogenic composition, or in some instances a vaccine formulation, to reduce atherosclerosis in hypercholesterolemic ApoE^{-/-} mice. We have also generated and characterized a humanized mouse model with chimeric HLA-A*02:01/Kb in ApoE^{-/-} background to test the efficacy of PAMs incorporating the P210 peptide (P210-PAMs) immunization as a bridge for future clinical testing. Class-I MHC/CD8⁺ T cell pathway is important in both the intrinsic immune response to P210 as well as potential immune-modulating therapy. HLA-A*02:01 is demonstrated herein as a prototype because the MHC-I allele occurs with the highest frequency in Western populations. Therefore, we have evaluated herein the effects of P210-PAM immunization on immune responses in atherosclerosis and tested the translational application of the P210-PAM formulation as a candidate human vaccine using HLA-A*02:01 transgenic mice. We have demonstrated that P210, when used in an active immunization strategy, elicited CD8⁺ T cell response to reduce atherosclerosis, potentially by shifting the immune-dominant epitope. These experimental observations implicate immune response to P210 in atherogenesis and indicate that modification of the intrinsic immune response to P210 could potentially reduce human atherosclerosis.

Various embodiments provide a peptide-amphiphile complex, which comprises, or consists of, a lipophilic or hydrophobic portion (e.g., tail) and a peptide portion (e.g., head group). In some embodiments, the peptide-amphiphile complex is one amphiphilic molecule, having a peptide (preferably hydrophilic peptide) that is covalently bonded to a lipid moiety or a lipophilic molecule; and hence the whole molecule comprises a peptide portion made up of the (hydrophilic) peptide, and a lipophilic portion made up of the lipid moiety or the lipophilic molecule, thereby the whole molecule being an amphiphilic molecule. In other embodiments, the peptide-amphiphile complex is a complex between a peptide (preferably hydrophilic peptide) and a lipophilic molecule, and hence resulting in an amphiphilic complex. In some aspects, the complex is a noncovalent bonding between the peptide and the lipophilic molecule. In other aspects, the complex is a covalent bonding between the peptide and the lipophilic molecule.

In some embodiments, the lipophilic portion is bonded to the peptide portion at the amino-terminal end of the peptide portion. Amino-terminal end is also referred to as the N-terminus. In other embodiments, the lipophilic portion is bonded to the C-terminus of the peptide portion. In yet another embodiment, the lipophilic portion is covalently linked to an amino acid residue of the peptide other than the N-terminal and C-terminal amino acid residues. In additional embodiments, a peptide-amphiphile complex has one or more lipophilic portions attached to the amino-terminal end, the C-terminus, and/or an amino acid residue of the peptide other than the N-terminal and C-terminal amino acid residues.

In various embodiments, hydrophilic or lipophilic property is in the context of a physiological environment, e.g., an environment that is in an aqueous medium, about physiological pH, and/or about physiological temperature. In some embodiments, a hydrophilic peptide has more than 50%, 60%, 70%, or more of its constituent amino acids being hydrophilic amino acids (e.g., arginine, asparagine, aspartate, glutamine, glutamate, histidine, lysine, serine, and threonine).

In various embodiments, the peptide portion preferably contains a biological function. Hence in various aspects, the peptide-amphiphile complex disclosed herein can be used for eliciting an immune response (e.g., a protective immune response), or for eliciting a therapeutic response, in a mammal, including human, against atherosclerosis or an ischemic cardiovascular disease. For example, the peptide portion comprises a recognition site by an antigen-presenting cell, so that an exemplary antigen-presenting cell, such as a dendritic cell, can uptake the peptide and optionally further present it as a self-peptide to T cells. In further aspects, the peptide is presented on the surface of a dendritic cell (after being uptaken by the dendritic cell), and/or it binds to a major histocompatibility complex (MHC) molecule (e.g., MHC-I, or MHC-II), so as to mediate stimulation of a T cell (e.g., CD8⁺ T cell; or elicit CD4 regulatory T cell response).

In some embodiments, the peptide portion includes no greater than about 30 amino acid residues. In some embodiment, the peptide portion comprises ApoB-100 derived peptide P210 having a sequence of KTTKQSFDLSVKAQYKKNKH (SEQ ID NO:1) or a fragment of SEQ ID NO:1 capable of binding a human leukocyte antigen (HLA) allele, or a variant of SEQ ID NO:1 having at least 95%, 94%, 93%, 92%, 91%, 90%, 85%, or 80% sequence identity to SEQ ID NO:1.

KTTKQSFDLSVKAQYKKNKH (SEQ ID NO:1) is ApoB-100 derived peptide P210 (ApoB-100 3136-3155), which can be synthesized or recombinantly produced.

A fragment of SEQ ID NO:1 capable of binding an HLA allele can be an epitope in the SEQ ID NO:1, which may be detected with a binding affinity to an HLA allele, such as a class-I HLA allele, or a class-II HLA allele. In some embodiments, the epitope of the SEQ ID NO:1 is a 5-, 6-, 7-, 8-, 9-, 10-, 11-, 12-, 13-, or 14-amino acid fragment of the SEQ ID NO:1. In some embodiments, the peptide-amphiphile complex comprises a peptide portion consisting of a fragment of SEQ ID NO:1, wherein the fragment is a 9-amino acid contiguous fragment of SEQ ID NO:1, such as any one of SEQ ID NOs:2-13.

In some embodiments, the P210 or its fragment or variant is in native form. In some embodiments, the P210 or its fragment or variant is in oxidized form, e.g., oxidized by exposure to copper. In some embodiments, the P210 or its fragment or variant is an aldehyde derivative, e.g., modified using malone dealdehyde (MDA). In some embodiments, the P210 or its fragment or variant is in the form of a hydroxynonenal-derivative thereof. In some embodiments, the P210 or its fragment or variant of ApoB-100 is a hapten of an aldehyde.

In various aspects, the lipophilic portion of the peptide-amphiphile complex typically does not detract from the structure of the peptide portion, and it may enhance and/or stabilize the structure of the peptide portion. In some situations, it may provide a hydrophobic surface for self-association (i.e., association without the formation of covalent bonds) and/or interaction with other surfaces. Thus, the lipophilic portion in complex with the hydrophilic peptide portion is also capable of forming a self-assembled structure, such as a micelle.

In some embodiments, the lipophilic portion can be any organic group having a long alkyl group, such as one having at least a branched group covalently coupled to at least two long alkyl groups, that are capable of forming lipid-like structures (e.g., with a hydrophilic peptide as a head, and the lipophilic portion as a tail). In some embodiments, the alkyl groups are linear chains, each having between 6-20, 10-18, or 12-16 carbon atoms in each chain; and/or this organic group also includes suitable functional groups for attachment to the peptide portion. In some embodiments, the lipophilic portion contains a trifunctional amino acid as a branched group, such as glutamate, so that two alkyl groups are each covalently bonded to the trifunctional amino acid and the trifunctional amino acid is further linked directly or indirectly to the peptide portion. In some embodiments, these alkyl groups are attached to the peptide portion through a linker group having suitable functionality such as ester groups, amide groups, and combinations thereof. Suitable lipophilic portions can be derived from compounds such as, for example, dialkylamines, dialkylesters, and phospholipids.

In some embodiments, the lipophilic portion being any organic group having an alkyl group includes a straight-chain, branched or cyclic, substituted or unsubstituted C₁-C₂₀ alkyl, C₂-C₂₀ alkenyl, C₂-C₂₀ alkynyl or aryl. In some embodiments, the lipophilic portion comprises two, three or more straight-chain, branched or cyclic, substituted or unsubstituted C₁-C₂₀ alkyl, C₂-C₂₀ alkenyl, C₂-C₂₀ alkynyl or aryl groups; and the lipophilic portion further contains or is conjugated to a multi-functional branching point (e.g., a multi-arm molecule having two or more functional groups for attachment). In some embodiments, the lipophilic portion comprises one, two, or three straight-chain, substituted or unsubstituted C₁-C₂₀ alkyl, C₂-C₂₀ alkenyl, C₂-C₂₀ alkynyl or aryl groups.

In various embodiments, any suitable covalent linkage is useful for attaching the lipophilic portion (e.g., lipid moiety, hydrophobic polymer) to the peptide. For example, the covalent linkage can be via an ester, amide, ether, thioether or carbon linkage. In some embodiments, the lipid moiety or hydrophobic polymer can be modified with a maleimide that reacts with a sulfhydryl group of the peptide, such as on a cysteine. In some embodiments, the lipid moiety or hydrophobic polymer can be linked to the peptide via click chemistry, by reaction of an azide and an alkyne to form a triazole ring. A number of other linkage strategies are known to those of skill in the art and can be used to synthesize the complex of the present invention. Such strategies are described in “Bioconjugate Techniques”, 2nd edition, G. T. Hermanson, Academic Press, Amsterdam, 2008.

The molecular weight of the lipid moiety or hydrophobic polymer can be chosen so as to tune the assembly and stability of the micelles. In general, the lipid moiety's molecular weight is sufficiently large to stabilize the assembled micelles but not so large as to interfere with the micelle assembly or presentation of the hydrophilic peptide.

In some embodiments, the peptide-amphiphile complex is a peptide-amphiphile molecule exemplified by a long chain dialkylester lipophilic (e.g., lipid) tail bonded to a peptide head group of the following formula:

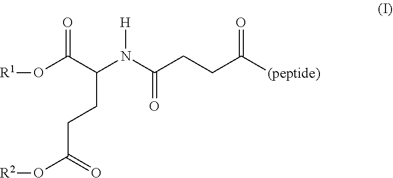

wherein:
(a) R¹ and R² are each independently C₁₀-C₂₀ or C₂₀-C₄₀ hydrocarbyl groups; and
(b) the (peptide) refers to a sequence of KTTKQSFDLSVKAQYKKNKH (SEQ ID NO:1), a fragment of SEQ ID NO:1 capable of binding a HLA, or a variant of SEQ ID NO:1 having at least 95%, 94%, 93%, 92%, 91%, 90%, 85%, or 80% sequence identity to SEQ ID NO:1.

In some embodiments, a peptide-amphiphile complex has a structure (II):

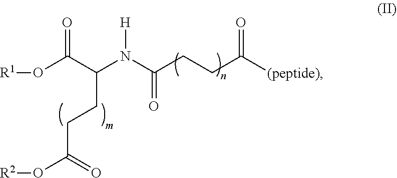

or a variant of (II), wherein the variant has any one or more of —O— or ═O in (II) BE independently substituted with S;
and wherein R¹ and R² are each independently C₆-C₂₀ (can be any integer between 6 and 20) substituted or unsubstituted hydrocarbyl groups; m and n are independently an integer (e.g., from 0 to 20) representing the number of repeats of unsubstituted or substituted —CH₂—CH₂— or ═O; and the (peptide) refers to a sequence of KTTKQSFDLSVKAQYKKNKH (SEQ ID NO:1), a fragment of SEQ ID NO:1 capable of binding an HLA, or a variant of SEQ ID NO:1 having at least 95%, 94%, 93%, 92%, 91%, 90%, 85%, or 80% sequence identity to SEQ ID NO:1.

In some embodiments, the peptide-amphiphile complex includes a detectable label, in the peptide portion, the lipophilic portion, or both. The detectable label can be a fluorophore, a chromogen, or an enzyme.

The complexes of the present invention can be made by a variety of solid-phase or solution techniques. For example, the peptides can be prepared by a solution method and then attached to a support material for subsequent coupling with the lipid; or more preferably, the peptides are prepared using standard solid-phase organic synthesis techniques, such as solid-phase peptide synthesis (SPPS) techniques. After coupling the peptide with a lipid, the peptide is then removed from a support material. Solid-phase peptide synthesis methods using functionalized insoluble support materials as well as removal afterwards are known in the art.

Various embodiments provide that the peptide-amphiphiles disclosed herein may form a structure in solution including micelles and vesicles. They can also be mixed with micelle/vesicle-forming lipids to form stable mixed micelles/vesicles. For example, mixed micelles can include suitable lipid compounds. Suitable lipids can include but are not limited to fats, waxes, sterols, cholesterol, fat-soluble vitamins, monoglycerides, diglycerides, phospholipids, sphingolipids, glycolipids, derivatized lipids, and the like. In some embodiments, suitable lipids can include amphipathic, neutral, non-cationic, anionic, cationic, or hydrophobic lipids. In certain embodiments, lipids can include those typically present in cellular membranes, such as phospholipids and/or sphingolipids. Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI). Non-cationic lipids include but are not limited to dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin. The lipids can also include derivatized lipids, such as PEGylated lipids. Optionally the micelle/vesicle-forming lipids may include a detectable label, such that the mixed micelles/vesicles formed together with a peptide-amphiphile disclosed herein is detectably labeled.

In some embodiments, a cylindrical micelle nanofiber is formed with a quantity of a peptide-amphiphile complex having a structure of:

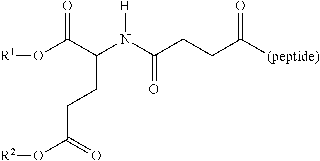

wherein: R¹ and R² are each independently C₁₀-C₂₀ hydrocarbyl groups; and the (peptide) refers to a sequence of KTTKQSFDLSVKAQYKKNKH (SEQ ID NO:1), a fragment of SEQ ID NO:1 capable of binding a HLA, or a variant of SEQ ID NO:1 having at least 95%, 94%, 93%, 92%, 91%, 90%, 85%, or 80% sequence identity to SEQ ID NO:1.

In some aspects the cylindrical micelle nanofiber has a circular cross-section with a diameter between 1-300 nm, 5-10 nm, or about 10-50 nm. In some aspects the cylindrical micelle nanofiber has a length that is at least two times or three times greater than the circular cross-section diameter; for example, the nanofiber may be at least about 70 nm, 80 nm, 90 nm, or 100 nm, or greater than 500 nm, 1 μm, or between 1-10 μm.

The micelles or vesicles based on the peptide-amphiphile complexes can be prepared by a thin film hydration method, typically including the steps of:
(a) drying a liquid film comprising the peptide-amphiphile complex having been dissolved in an organic solvent (e.g., methanol), to result in a lipid film comprising the peptide-amphiphile complex; optionally, the drying includes evaporation under a nitrogen or inert gas flow;
(b) hydrating the lipid film comprising the peptide-amphiphile complex in an aqueous medium (e.g., water, or optionally in combination with buffering salts or the like), wherein the aqueous medium is heated to a temperature (e.g., above a gel-liquid crystal transition temperature of the peptide-amphiphile complex), thereby obtaining a hydrated lipid suspension comprising the peptide-amphiphile complex; and
(c) subjecting the hydrated lipid suspension comprising the peptide-amphiphile complex to sonication or extrusion, so as to obtain a micellar composition composed of the peptide-amphiphile complex.

Gel-liquid crystal transition temperature may be determined by differential scanning calorimetry (DSC).

In some embodiments, the step of hydrating the lipid film in a heated temperature is hydrating the lipid film at a temperature above the phase transition temperature of lipid or lipid-like constituent of the lipophilic portion of the peptide-amphiphile complex. Transition temperatures (or phase transition temperatures) of various lipids or glycerophospholipids are known in the art, and can be accessed via one or more database such as avantilipids.com/tech-support/faqs/transition-temperature.

Pharmaceutical compositions are also provided, including a peptide-amphiphile complex disclosed herein, or a micelle/vesicle formed therefrom, and a pharmaceutically acceptable excipient. Pharmaceutically acceptable excipients may be carriers, innocuous fillers and/or adjuvants.

Immunogenic compositions are also provided, which can be used for eliciting an immune response in a mammal having an ischemic cardiovascular disease. In other embodiments, immunogenic compositions are used for eliciting an immune response against atherosclerosis or an ischemic cardiovascular disease in a subject. Vaccine compositions are also provided for immunization of a mammal including human against an ischemic cardiovascular disease. In some embodiments, the immunogenic composition is a vaccine composition, and the immune response is a protective immune response. In various embodiments, a vaccine composition includes an active component (e.g., a peptide-amphiphile complex, especially micellar nanoparticles formed of the peptide-amphiphile complex) which induces the immune response. In some embodiments, a vaccine composition may include a peptide-amphiphile complex in an amount, for example, ranging from 0.1 μg to 100 mg. A vaccine composition may also contain additional components such as preservatives, additives, adjuvants, carrier, and traces of other components. Examples of adjuvants comprise adjuvants having Th2 effects, carriers having adjuvant properties, e.g., diphtheria toxoid, and adjuvants able to function as carriers, e.g., oil-water emulsions.

Eliciting protective immune response can refer to inducing the production and presence of circulating antibody against the peptide-amphiphile complex (humoral immunity), the actions of sensitized T-lymphocytes (cell-mediated immunity), and the production and presence of secretory IgA on mucosal surface (mucosal immunity), or a combination of these factors; which typically provides protection of the subject prior to occurrence of diseases (e.g., toxin-induced diseases) or viral or bacterial infections, and/or prior to recurrence of the disease.

In other embodiments, the immune response is a therapeutic response and can treat the ischemic cardiovascular disease. The immunogenic compositions include a therapeutically effective amount of a peptide-amphiphile complex disclosed herein, optionally in combination with an adjuvant.

In some embodiments, the immunogenic compositions or the vaccines include a therapeutically effective amount of micelles or vesicles formed from the peptide-amphiphile complex, optionally in combination with an adjuvant. In some embodiments, the peptide-amphiphile complex does not include, or is not co-administered with, an MHC molecule. The MHC molecules are glycoproteins encoded in a large cluster of genes located on chromosome 6, which have potent effect on the immune response; and in humans, these genes are often called human leukocyte antigens (HLAs). MHC is the term for the region located on the short arm of chromosome 6p21.31 in humans and chromosome 17 in mice. For example, the MHC-I region in the chromosome encodes HLA antigens of HLA-A, -B, and -C; the MHC-II region encodes HLA antigens of HLA-DR, -DQ, and -DP; ad the MHC-III region includes several genes involved in the complement cascade (C4A, C4B, C2, and FB). Hence, in some embodiment, the peptide-amphiphile complex does not include, or is not co-administered with, an HLA antigen when formulated for use in a human subject.

Some embodiments provide methods for eliciting an immune response in a subject having atherosclerosis or an ischemic cardiovascular disease, wherein the methods include administering to the subject a pharmaceutical composition including a therapeutically effective amount of micelles or vesicles formed from the peptide-amphiphile complex disclosed herein, or administering to the subject the immunogenic composition disclosed herein.

Some embodiments provide methods for treating, reducing severity, or inhibiting progression of atherosclerosis or an ischemic cardiovascular disease in a subject in need thereof, wherein the methods include administering to the subject a pharmaceutical composition including a therapeutically effective amount of micelles or vesicles formed from the peptide-amphiphile complex disclosed herein, or administering to the subject the immunogenic composition disclosed herein.

Additional embodiments provide methods for eliciting an immune response or therapeutic treatment of a subject against atherosclerosis or an ischemic cardiovascular disease, wherein the methods include administering to the subject a pharmaceutical composition including a therapeutically effective amount of micelles or vesicles formed from the peptide-amphiphile complex disclosed herein, or administering to the subject the immunogenic composition disclosed herein.

In some embodiments, the therapeutically effective amount reduces cytolytic activity of CD8⁺ T cell, reduces proliferative activity of CD4⁺ T cell, and/or reduces aortic atherosclerosis in the subject.

In some embodiments, a method of increasing CD8⁺ T cells in a human subject with atherosclerosis or acute coronary syndrome is provided, which includes administering to the subject a pharmaceutical composition comprising KTTKQSFDLSVKAQYKKNKH (SEQ ID NO:1) or a fragment of SEQ ID NO:1 capable of binding a human leukocyte antigen (HLA). In other embodiments, a method of increasing CD8⁺ T cells in a human subject with atherosclerosis or acute coronary syndrome is provided, which includes administering to the subject a pharmaceutical composition comprising a peptide-amphiphile complex disclosed herein, or a pharmaceutical composition comprising micelle nanoparticles formed from a peptide-amphiphile complex disclosed herein.

In some embodiments, the increased CD8⁺ T cells comprises CD8⁺ T effector cells, CD8⁺ T effector memory cells, or both. In some embodiments, the increased CD8⁺ T cells in the human subject after the administration is compared to the amount of the CD8⁺ T cells in the human subject having the atherosclerosis or acute coronary syndrome but before the administration. In various aspects, a human subject having atherosclerosis or acute coronary syndrome have underlying atherosclerotic vascular disease. In some embodiments, the increased CD8⁺ T cells in the human subject after the administration is compared to the amount of the CD8⁺ T cells in a healthy human subject administered with the pharmaceutical composition.

In some embodiments, a method is provided for reducing atherosclerosis or aortic atherosclerosis in a mammalian subject, preferably human, and the method includes administering to the subject a pharmaceutical composition comprising a peptide-amphiphile complex disclosed herein, or a pharmaceutical composition comprising micelle nanoparticles formed from a peptide-amphiphile complex disclosed herein, wherein the mammalian subject has or is diagnosed with hypercholesterolemia.

In some embodiments, a method is provided for diminishing CD4⁺ T cell proliferation, reducing CD8⁺ T cell cytolytic activity, or both, which includes administering to the subject a pharmaceutical composition comprising a peptide-amphiphile complex disclosed herein, or a pharmaceutical composition comprising micelle nanoparticles formed from a peptide-amphiphile complex disclosed herein, wherein the mammalian subject has or is diagnosed with hypercholesterolemia.

In some embodiments, the subject in need of an immune response or a therapeutic treatment or prophylaxis is one suffering from atherosclerosis. In some embodiments, the subject in need thereof is one having acute coronary syndrome. In some embodiments, the subject in need thereof is one detected with or having an ischemic cardiovascular disease. In further embodiment, the subject suffering from atherosclerosis, having acute coronary syndrome, or having an ischemic cardiovascular disease is a human.

In other embodiments, the subject in need thereof does not have atherosclerosis, acute coronary syndrome, or an ischemic cardiovascular disease at the time of the administration. Hence the provided composition is an immunogenic composition, which may be used in eliciting a protective immune response in the subject against atherosclerosis, acute coronary syndrome, or an ischemic cardiovascular disease.

In some embodiments, the therapeutically or prophylactically effective amount of the peptide-amphiphile complex (or the micelles/vesicles composed of the peptide-amphiphile complex) is any one or more of about 0.01 to 0.05 μg/kg of subject/dose, 1 to 5 μg/kg of subject/dose, 5 to 10 μg/kg of subject/dose, 10 to 20 μg/kg of subject/dose, 20 to 50 μg/kg of subject/dose, 50 to 100 μg/kg of subject/dose, 100 to 150 μg/kg of subject/dose, 150 to 200 μg/kg of subject/dose, 200 to 250 μg/kg of subject/dose, 250 to 300 μg/kg of subject/dose, 300 to 350 μg/kg of subject/dose, 350 to 400 μg/kg of subject/dose, 400 to 500 μg/kg of subject/dose, 500 to 600 μg/kg of subject/dose, 600 to 700 μg/kg of subject/dose, 700 to 800 μg/kg of subject/dose, 800 to 900 μg/kg of subject/dose, 900 to 1000 μg/kg of subject/dose, 0.01 to 0.05 mg/kg of subject/dose, 0.05-0.1 mg/kg of subject/dose, 0.1 to 0.5 mg/kg of subject/dose, 0.5 to 1 mg/kg of subject/dose, 1 to 5 mg/kg of subject/dose, 5 to 10 mg/kg of subject/dose, 10 to 15 mg/kg of subject/dose, 15 to 20 mg/kg of subject/dose, 20 to 50 mg/kg of subject/dose, 50 to 100 mg/kg of subject/dose, 100 to 200 mg/kg of subject/dose, 200 to 300 mg/kg of subject/dose, 300 to 400 mg/kg of subject/dose, 400 to 500 mg/kg of subject/dose, 500 to 600 mg/kg of subject/dose, 600 to 700 mg/kg of subject/dose, 700 to 800 mg/kg of subject/dose, 800 to 900 mg/kg of subject/dose, 900 to 1000 mg/kg of subject/dose or a combination thereof. In some aspects, the method includes one dose of the peptide-amphiphile complex (or micelles/vesicles composed of the peptide-amphiphile complex). In some aspects, the method includes two or more doses of the peptide-amphiphile complex (or micelles/vesicles composed of the peptide-amphiphile complex), with adjacent doses being at least one week, two weeks, one month, two months, three months, four months, five months, or six months apart. In some aspects, the method includes a primary dose followed by one or more booster doses, wherein the booster doses may be one week, two weeks, three weeks, four weeks, one month, two months, three months, four months, five months, six months, or more after an immediate previous dose.

In some embodiments, the pharmaceutical composition, micellar composition, and/or vaccine composition based on the peptide-amphiphile complex is administered subcutaneously. In some embodiments, the pharmaceutical composition, micellar composition, and/or vaccine composition based on the peptide-amphiphile complex is administered intramuscularly. In other embodiments, the pharmaceutical composition, micellar composition, and/or vaccine composition based on the peptide-amphiphile complex is administered via another route of choice.

In some embodiments, the pharmaceutical composition, micellar composition, and/or vaccine composition of the present invention may also be formulated into a solution, a solid preparation or a spray, and suitably use, if desired, excipient, binder, perfume, flavor, sweetener, colorant, preservative, antioxidant, stabilizer, surfactant, and/or the like, in addition to the materials described above.

In some embodiments, the pharmaceutical composition, micellar composition, and/or vaccine composition is administered locally at an injection site to the subject; and at least 50% of the peptide-amphiphile complex in the pharmaceutical composition remains near the injection site 2 days following the administration, at least 10% of said peptide-amphiphile complex remains near the injection site 5 days following the administration, and/or at least 5% of said peptide-amphiphile complex remains near the injection site 7 days following the administration; said nearing to the injection site being within ±30 mm from the injection site.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Intrinsic T Cell Response to ApoB-100 Peptide P210 in ACS Patients

Our previous reports demonstrated that immune modulation of T cells reactive with the ApoB-100 peptide P210 in the ApoE^{-/-} mice reduced atherosclerosis. To evaluate if self-reactive T cell response to P210 is present in humans, we investigated the intrinsic T cell response to P210 in humans by testing peripheral blood mononuclear cells (PBMCs) from acute coronary syndrome (ACS) patients and self-reported healthy volunteers as controls. ACS patients were selected for this exploratory study because of unequivocal ASCVD in these subjects. Patient characteristics are in Table 1.

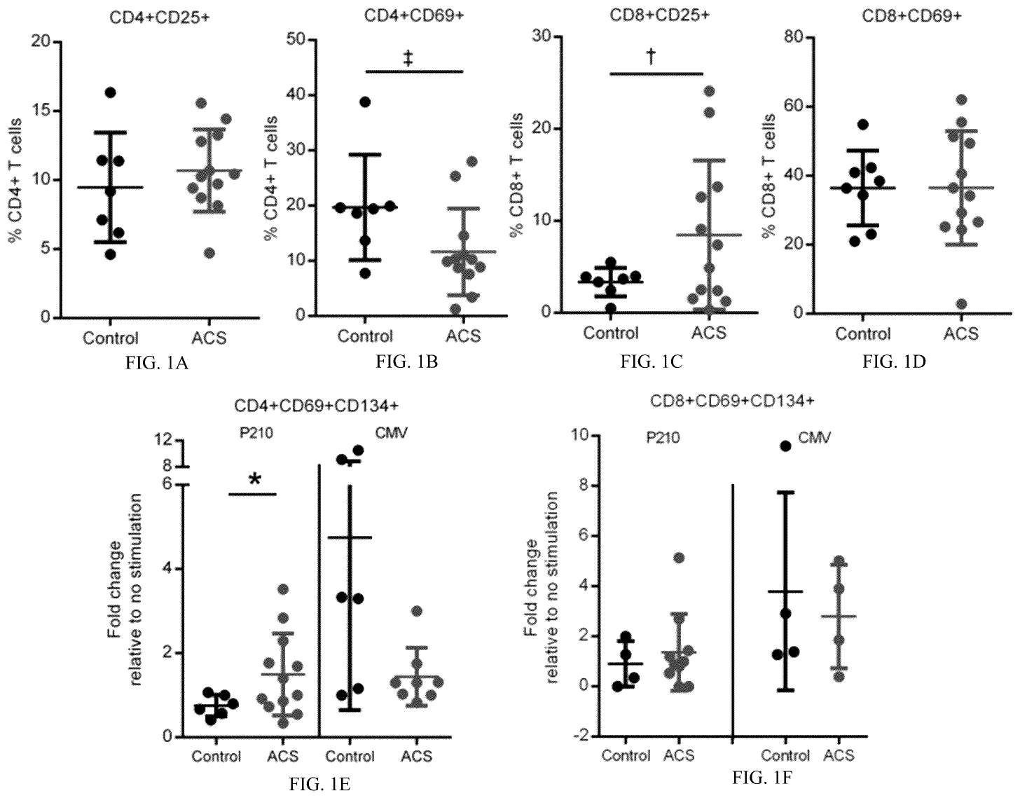

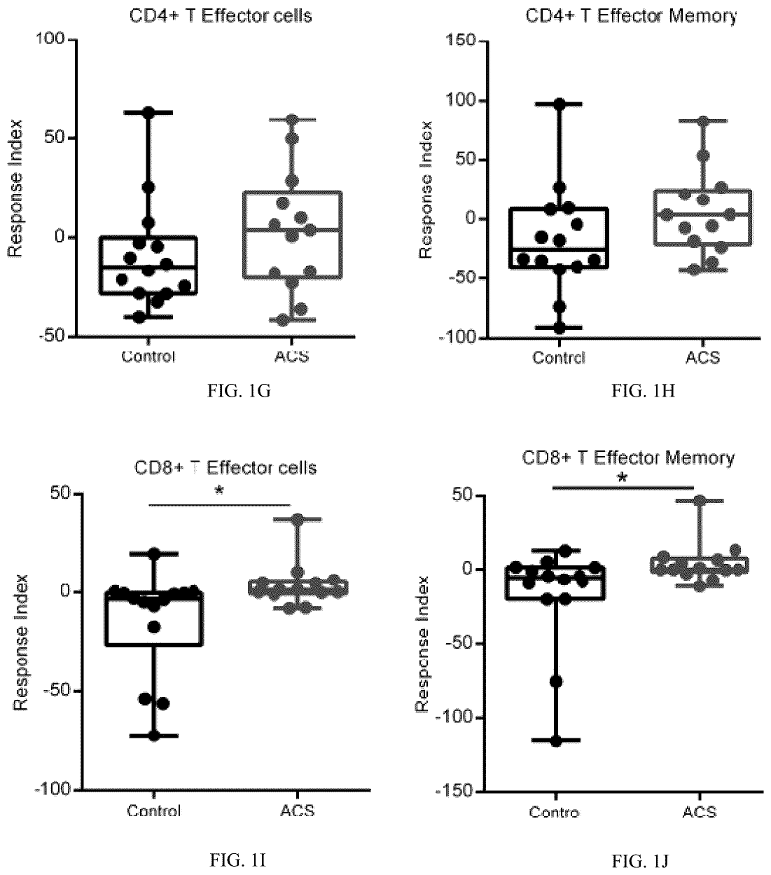

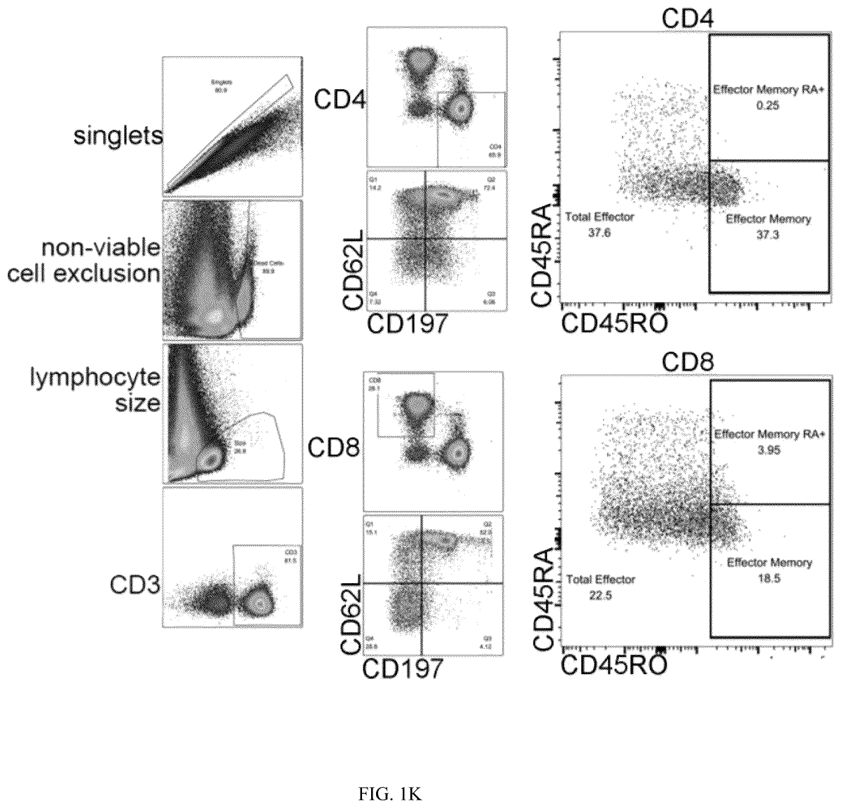

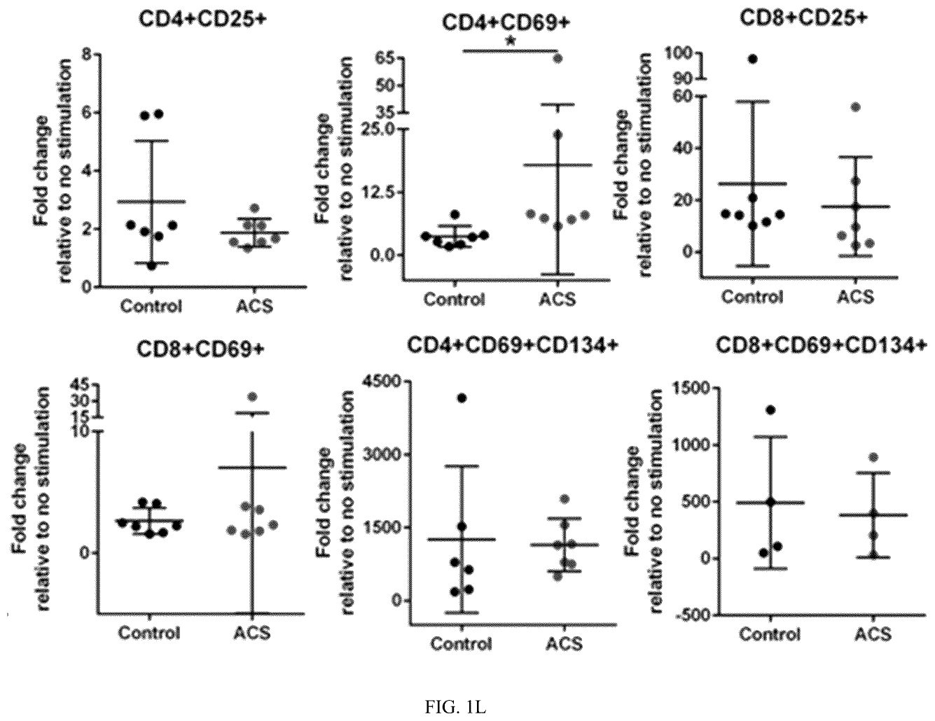

In order to determine if P210 is capable of activating T cells as an antigen, we conducted an Activation Induced Marker (AIM) assay. At baseline, there were fewer CD4⁺CD69⁺ T cells and greater CD8⁺CD25⁺ T cells in PBMCs from ACS patients compared to control subjects, whereas no difference in CD4⁺CD25+ and CD8⁺CD69⁺ T cells between 2 groups were noted (FIG. 1A-1D, P=0.07 for FIG. 1B, P=0.05 for FIG. 1C). AIM assays demonstrated a mean 1.5-fold increase in CD4⁺CD69⁺CD134⁺ T cells after P210 stimulation in ACS patients compared to control subjects while no such increase was observed in CD8⁺CD69⁺CD134⁺ T cells (FIG. 1E, 1F). CMV pooled peptide (right panel in FIG. 1E, 1F) or cell stimulation cocktail (PMA/Ionomycin, FIG. 1L) as positive controls validated the AIM assay.

We did not observe differences in CD25⁺CD134⁺, CD69⁺CD154⁺ or CD134⁺CD137⁺ in either CD4⁺ or CD8⁺ T cells (FIG. 1M). Although a cut-off of 2-fold increase may be appropriate in studying T cell activation to exogenous antigens (infectious or vaccine antigens), T cell responses to intrinsic self-antigens are not expected to be as robust, since the immune-inflammatory response to self-antigens in auto-immune diseases tend to be chronic and low grade.

A hallmark feature of adaptive immune response is the recall response of antigen-experienced T cells to antigen re-exposure. Given ACS patients have definite atherosclerosis, we tested if T cells from ACS patients would generate such recall response to P210 restimulation. CD4⁺ T effector cell response to P210 was not significantly different in the ACS PBMCs compared to controls (FIG. 1G & 1H). However, there was a significant increase in CD8⁺ T effector (FIG. 1I), and CD8⁺ T effector memory (FIG. 1J; gating strategy for T cells in FIG. 1K) response in ACS PBMCs compared to controls, which supports the existence of antigen-experienced, P210-specific T cells in humans with atherosclerosis.

Characteristics of P210 Peptide

The T cell response observed in PBMCs from ACS patients indicated that P210 may be a self-peptide that provokes a self-reactive immune response. It remains unknown how apolipoprotein B-100 (ApoB-100) peptides become immunogenic, but the presence of antibodies against ApoB-100 peptides in patients with ASCVD indicates the potential of antigen presenting cells (APC) to present peptides derived from LDL particles that have undergone oxidation and subsequent breakdown. Indeed, various ApoB-100 peptide fragments, including P210, have been detected in atherosclerotic plaques by mass spectrometry (Mayr, M. et al., Circ. Cardiovasc. Genet. 2009, 2:379-388). However, it remains unknown how ApoB-100 peptides, specifically P210, are able to enter dendritic cells (DCs) to function as intrinsic self-antigens.

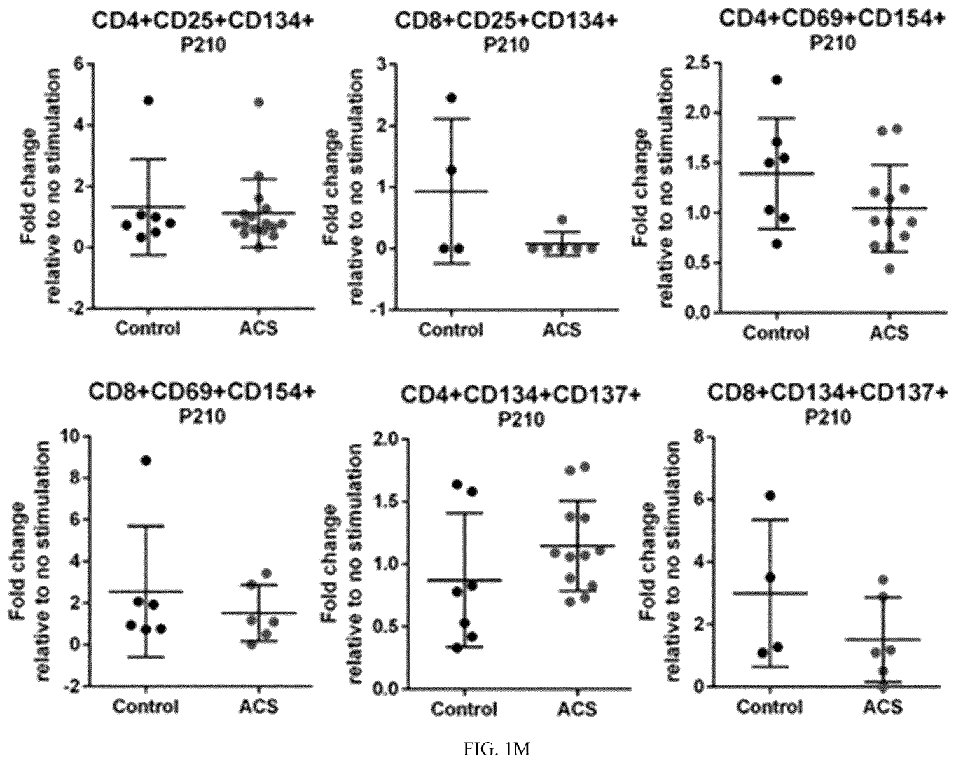

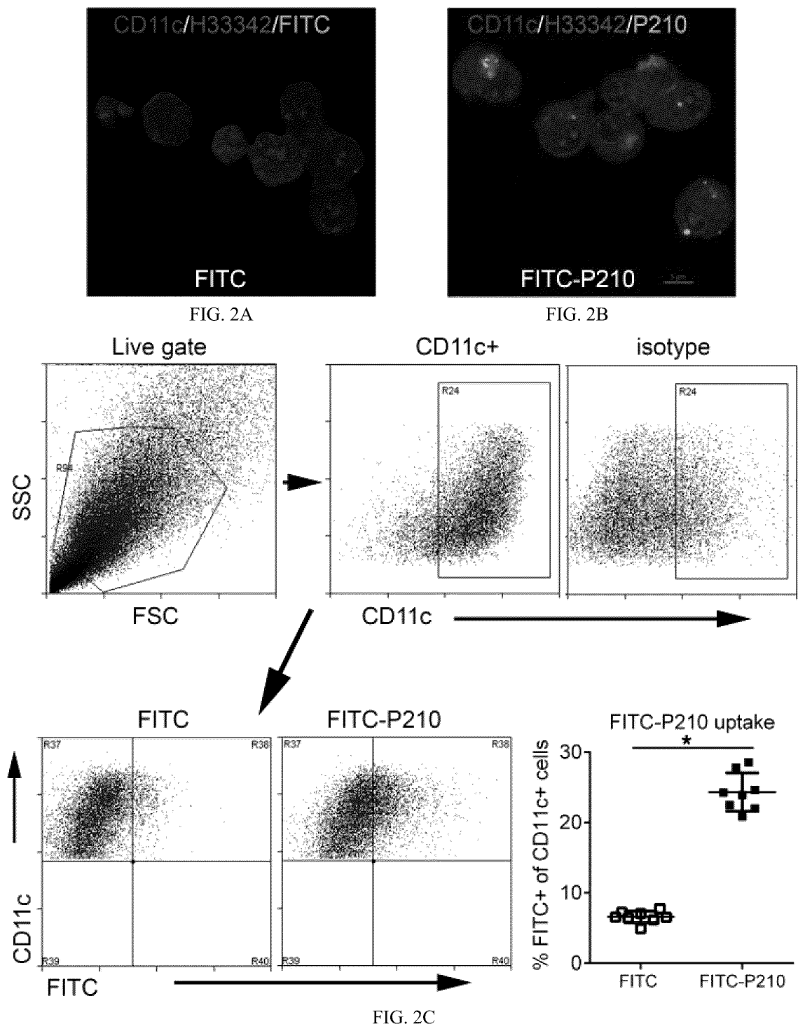

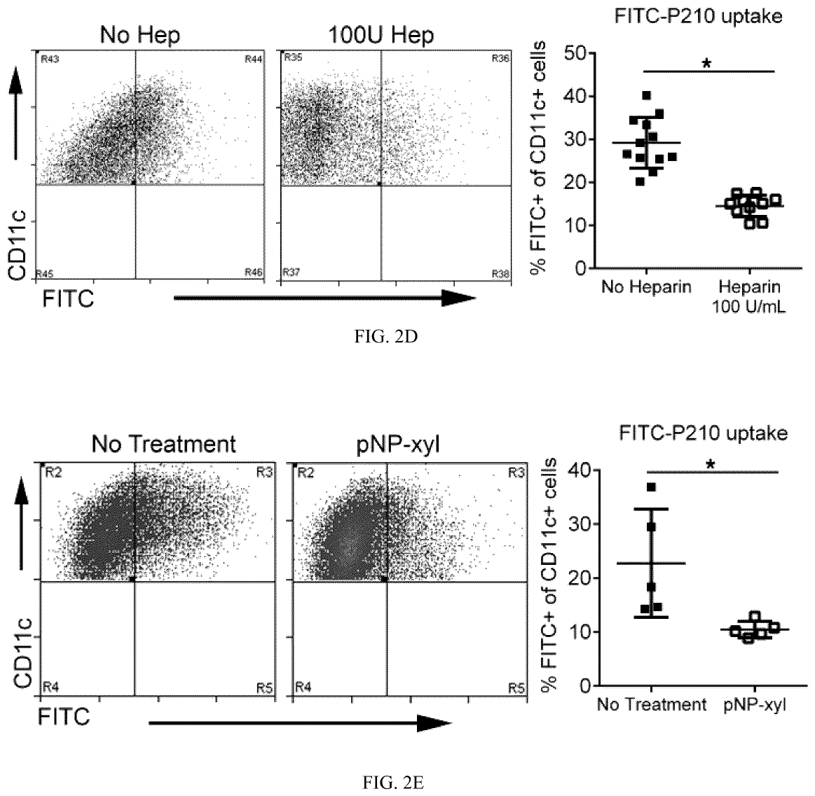

P210 is a cationic peptide fragment that is within the proteoglycan binding domain of ApoB-100 that has the properties of a cell-penetrating peptide (CPP). Cationic CPPs are rich in positively charged Arg and Lys residues, which allows for interaction with negatively charged cell surface proteoglycans. Given the Lys-rich sequence of P210 (KTTKQSFDLSVKAQYKKNKH (SEQ ID NO:1)) and a calculated isoelectric point (p1) of 10.85, we investigated if P210 could enter mouse bone marrow-derived dendritic cells (DCs) through the proteoglycan pathway. To test this, we used confocal microscopy to visualize the uptake of FITC-conjugated P210 peptides (P210-FITC) into CD11c⁺ DCs, and flow cytometric analysis confirmed significantly increased uptake of P210-FITC (FIG. 2A-2C). The proteoglycan binding capacity of P210 was assessed by using heparan to block DC uptake, and P210-FITC entry into DCs was significantly reduced by 100 U/ml of heparan (FIG. 2D). To further confirm that the cellular uptake of P210 is mediated by cell surface proteoglycan binding, DCs were treated with p-nitrophenyl-O-D-xylopyranoside (pNP-xyl), a competitive inhibitor of heparan sulfate chain addition, preventing the synthesis of functional cell surface heparan sulfate proteoglycans. Treatment of DCs with pNP-xyl significantly reduced P210-FITC entry (FIG. 2E), supporting the notion that P210 uptake by DCs is mediated in part through cell surface proteoglycan binding. The results demonstrate that P210 has properties of a CPP that enables its entry into APCs such as DCs and potentially presented to T cells as a self-peptide.

Immune Modulation and Biodistribution of P210 Nanoparticles

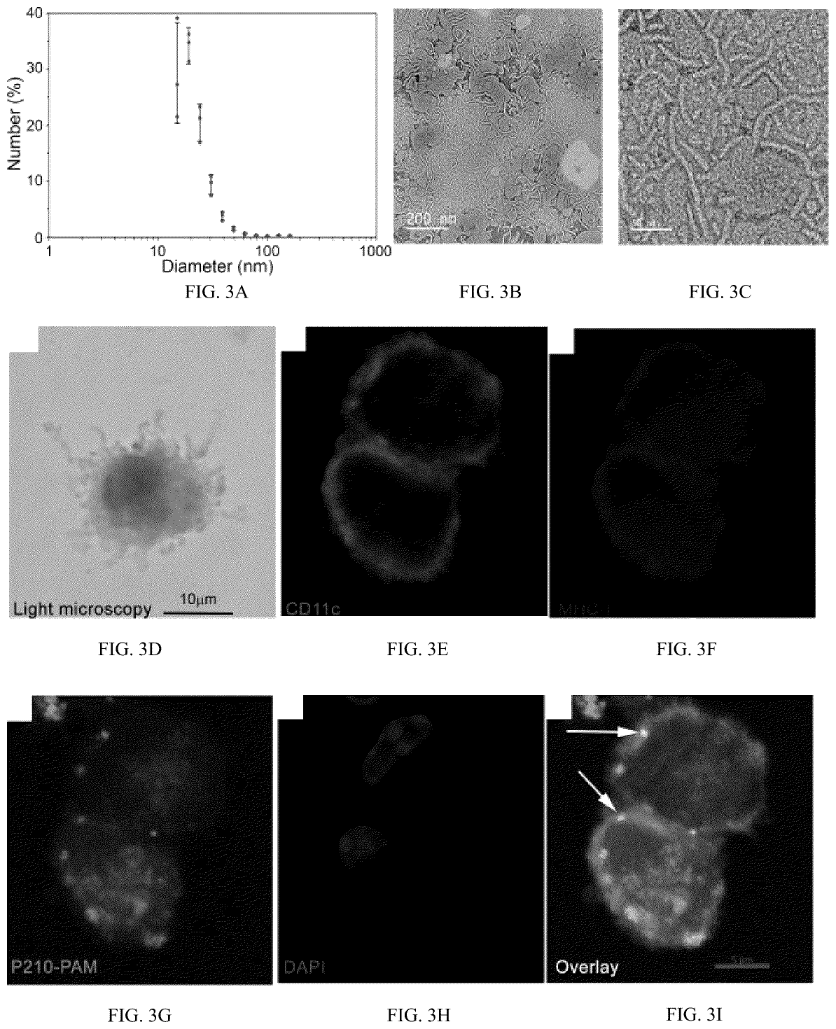

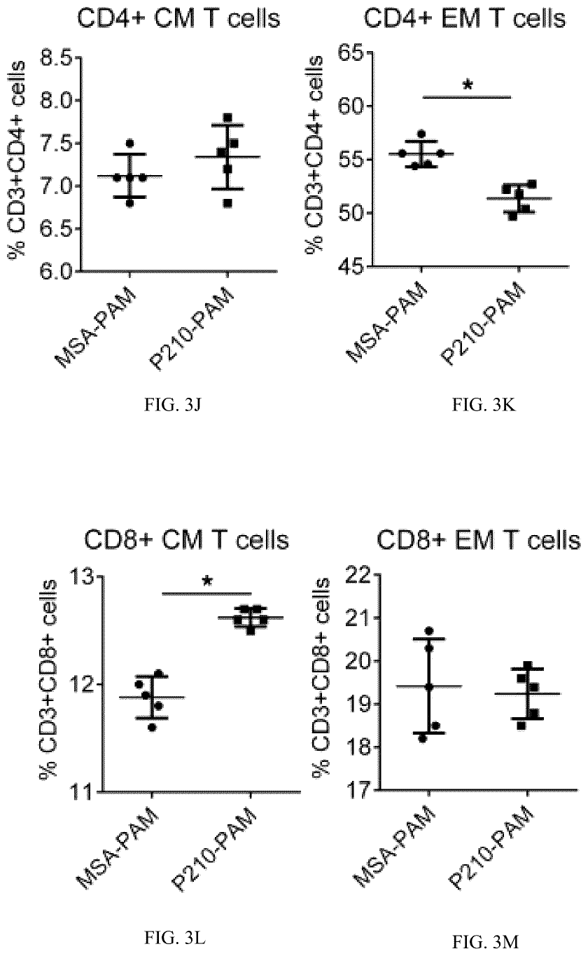

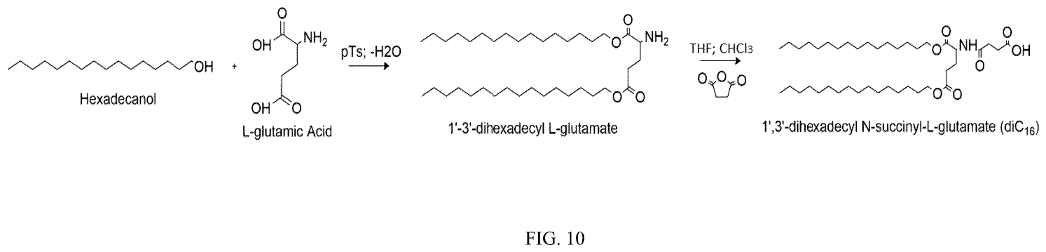

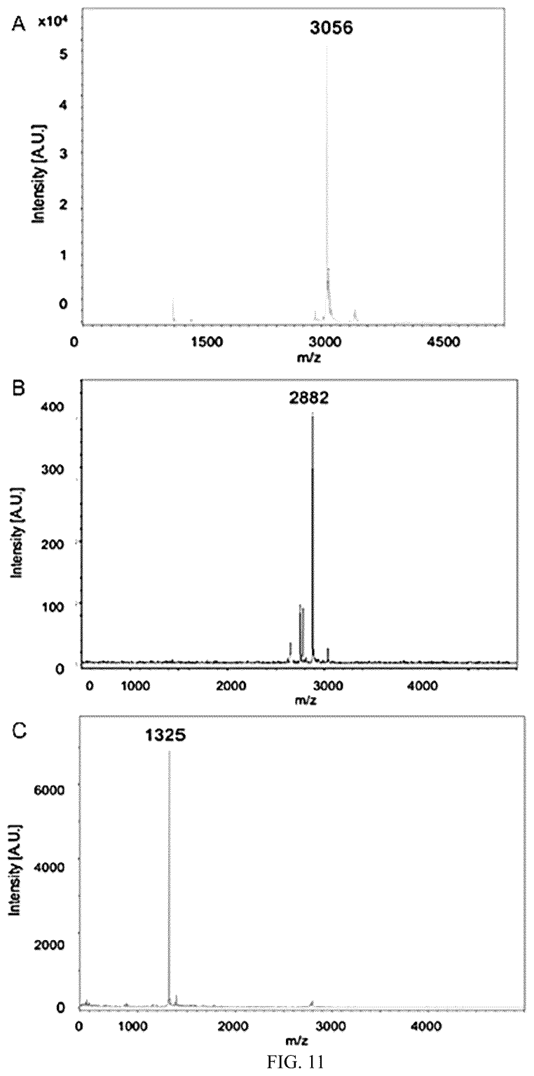

To enable efficient antigen delivery by protecting peptides from protease degradation and clearance and providing a scaffold for increased epitope density, P210 was incorporated into peptide amphiphile micelles (PAMs) through covalent conjugation of the peptide to 1′-3′-dihexadecyl N-succinyl-L-glutamate (diC₁₆) hydrophobic moieties. Hydrophobic interaction induced self-assembly of the diC₁₆-P210 monomers into cylindrical micelles with an average diameter of 21.6±1.1 nm, a polydispersity index of 0.152±0.001 and a zeta potential of 2.7±0.8 mV (FIG. 3A-3C, FIG. 10 and FIG. 11). An average length measured from ten representative PAMs from the T_{EM} image is 87.3±35.9 nm.

First, we tested whether P210-PAM enters DCs and if P210 (or its fragment) can be contained with MHC-I by conducting confocal experiments using FITC-labeled P210-PAM. MHC-I was chosen as the pathway to visualize given prior data indicating the involvement of MHC-I/CD8+ T cell pathway in P210 immunization, consistent with the reported characterization of CPPs to be cross-presented to MHC-I. Confocal microscopy demonstrated costaining of FITC-labelled P210 with MHC-I molecule on the surface of mouse DCs (FIG. 3E-3I).

P210-PAM was then tested for reactivity with T cells of ApoE^{-/-} mice and Mouse Serum Albumin peptide amphiphile micelles (MSA-PAM) were used as a control. There was a significant reduction in CD4+ effector memory T cells and increase in CD8+ central memory T cells treated with P210-PAM when compared to MSA-PAM treated splenocytes of ApoE^{-/-} mice fed high cholesterol diet for 16 weeks (FIG. 3K, 3L). Although central memory CD4⁺ T cells and effector memory CD8⁺ T cells remained unchanged (FIG. 3J, 3M), the results suggest that P210-PAM provokes a memory T cell response in naïve hypercholesterolemic ApoE^{-/-} mice.

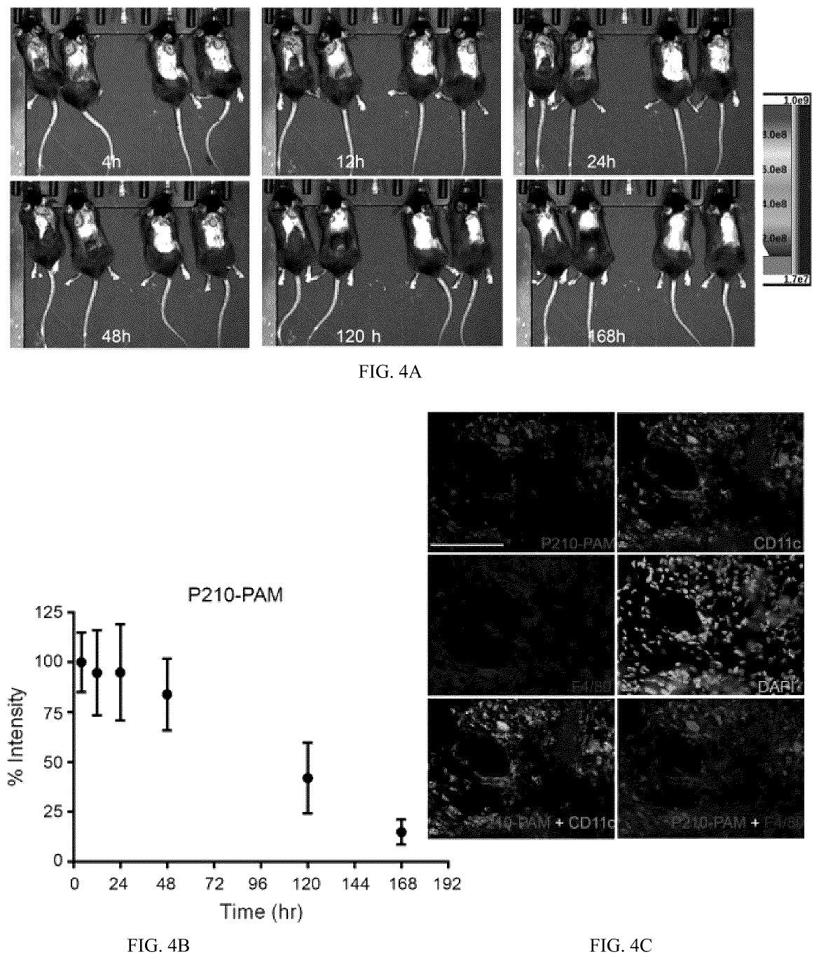

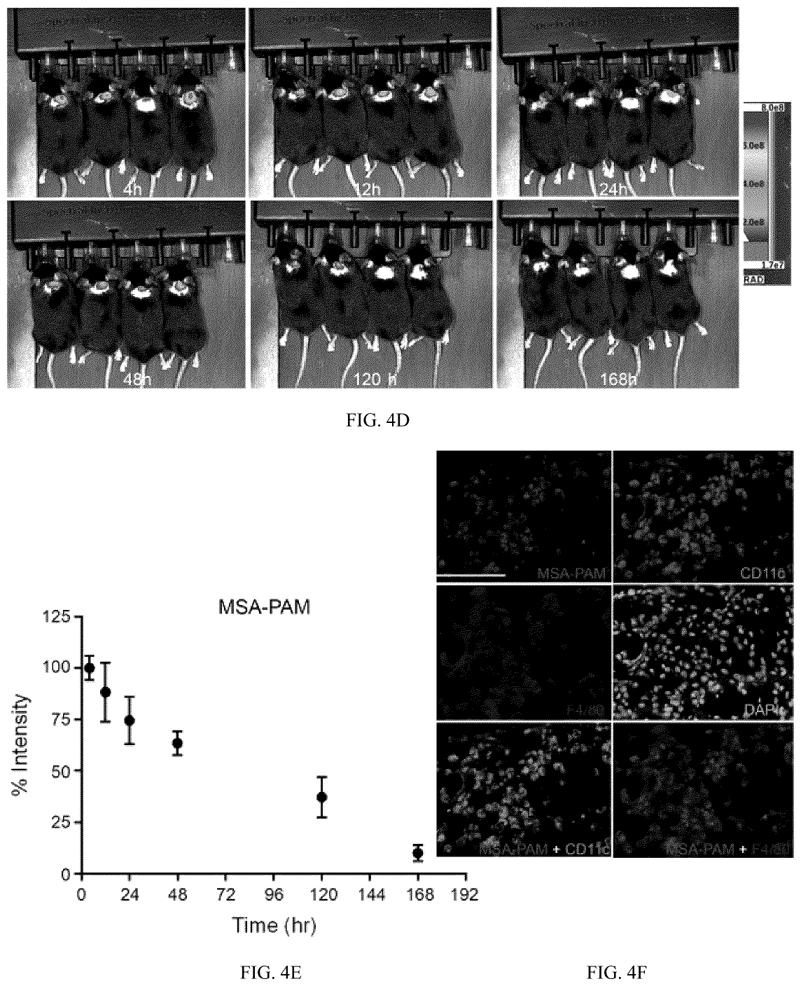

Effective immunization depends not only on the immunogenicity of antigens but also on their retention at the injection site. We hence characterized the biodistribution kinetics of fluorescently labeled P210-PAM injected subcutaneously in wild type mice and imaged over a period of 7 days, showing 80%, 30% and 15% retention in the injection site at 2, 5 and 7 days, respectively with a calculated clearance half-life 79.7±29.2 hrs (FIG. 4A, 4B). Immuno-fluorescent staining of the injection site showed colocalization of P210-PAM with F4/80⁺ macrophages and CD11c⁺ DCs (FIG. 4C). MSA-PAM had percent retention of 67%, 37%, and 11% at 2, 5, and 7 days, respectively, and the clearance half-life of 72.7±29.2 hrs (FIG. 4D-4F).

Nanoparticle-Based Immune Modulation of T Cell Responses to P210-PAM Immunization

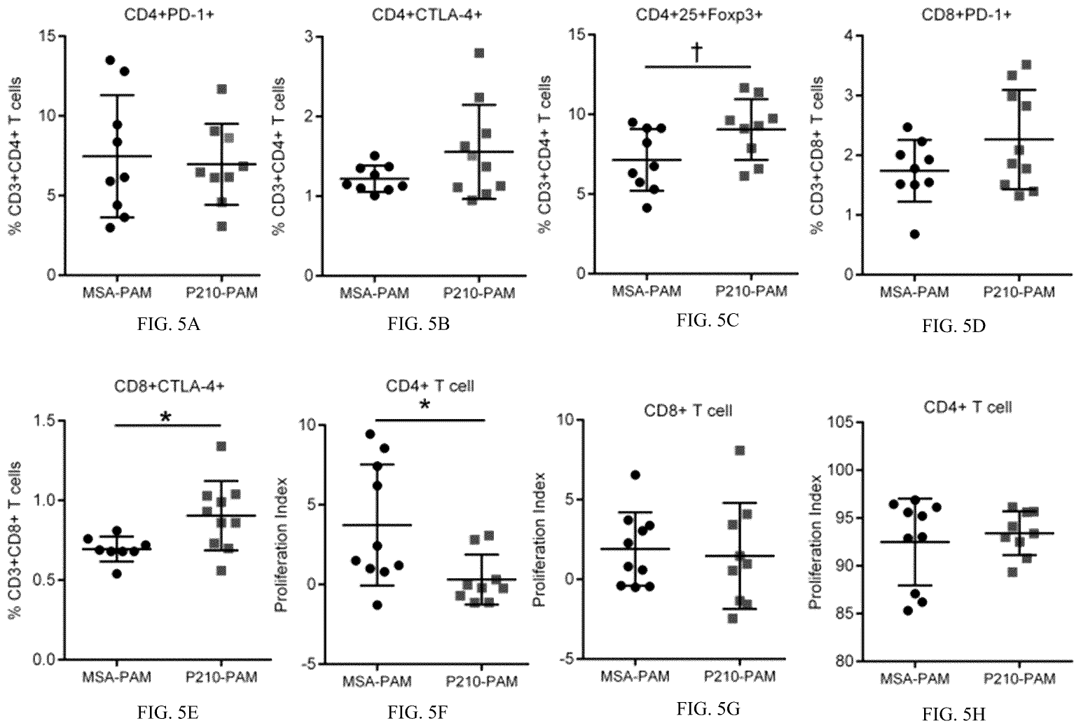

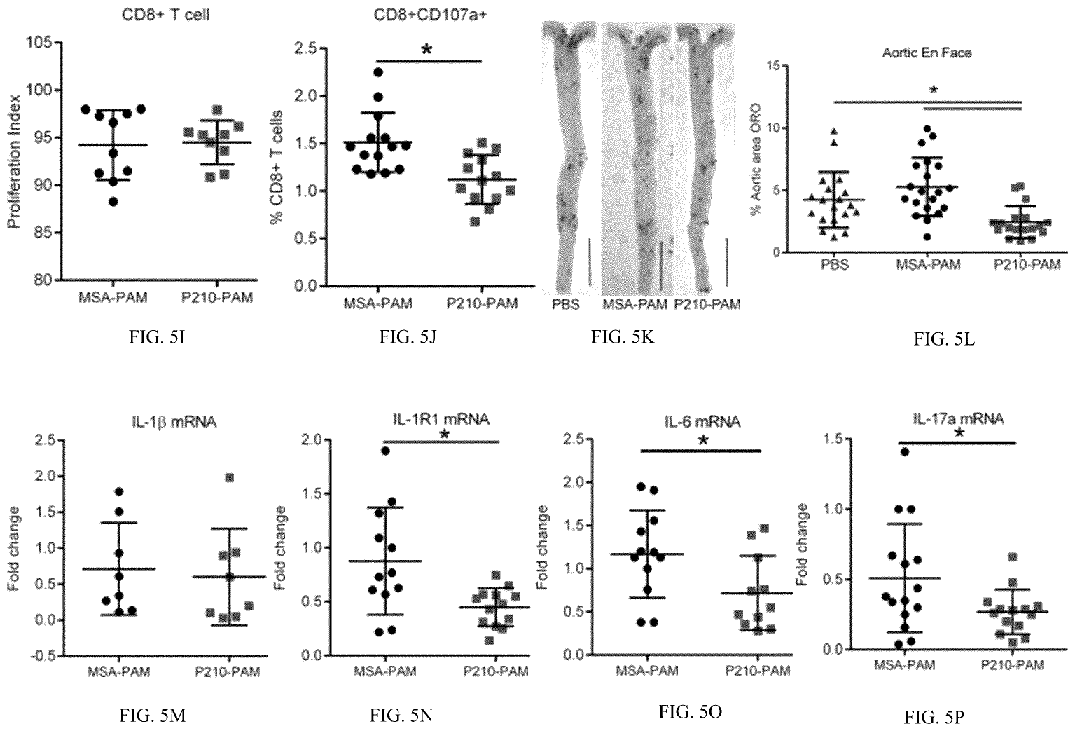

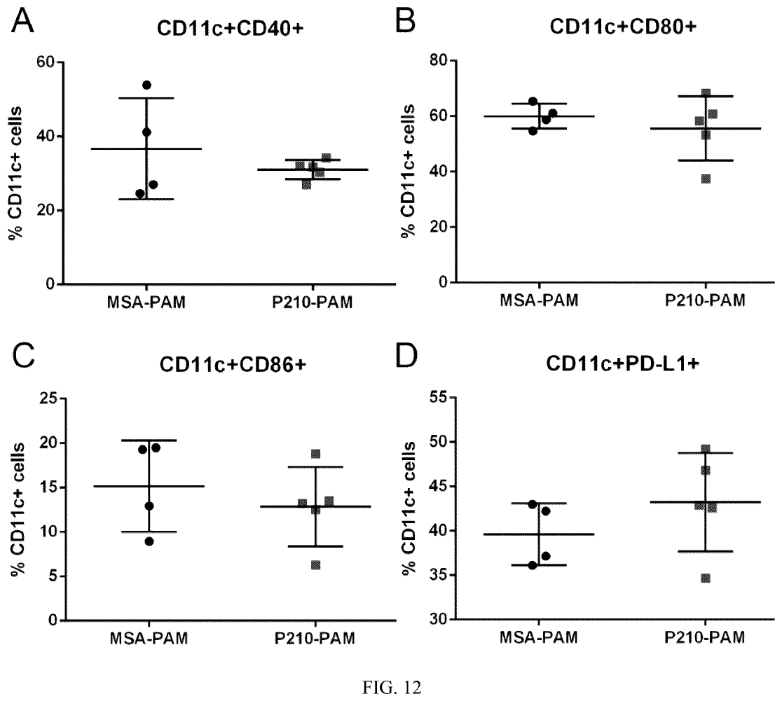

The effect of P210-PAM immunization on immune regulation was then tested in ApoE^{-/-} mice using the MSA-PAM as a control. Immunized male ApoE^{-/-} mice euthanized 1 week after the second booster injection showed no differences in splenic CD4⁺PD-1⁺ and CD4⁺CTLA-4⁺ T cells between P210-PAM and MSA-PAM immunized mice (FIG. 5A, 5B). There was increased CD4⁺CD25⁺FoxP3⁺ T_{reg} cells in P210-PAM immunized mice compared to those immunized with MSA-PAM (FIG. 5C, P=0.05). There were no differences in CD8⁺PD-1⁺ T cell numbers (FIG. 5D) but CD8⁺CTLA-4⁺ T cells were significantly increased in P210-PAM immunized mice compared to MSA-PAM immunized mice (FIG. 5E). CD4⁺ T cells from P210-PAM immunized mice had significantly reduced proliferative response to P210 stimulation compared to CD4⁺ T cells from MSA-PAM immunized mice (FIG. 5F), but this was not observed in CD8⁺ T cells (FIG. 5G). CD4⁺ T cells (FIG. 5H) and CD8⁺ T cells (FIG. 5I) responded to concanavalin A (Con A) stimulation similarly between the 2 groups indicating specificity of the regulation of T cell response. Even though P210-PAM immunization had no effect on CD8⁺ T cells proliferation, there was reduced cytolytic function of CD8⁺ T cells in response to P210 stimulation in P210-PAM immunized mice compared to MSA-PAM immunized mice as determined by CD107a staining (FIG. 5J). Thus, P210-PAM provoked antigen-specific effects as well as regulation of CD4⁺ T cells' proliferation and CD8⁺ T cells' cytolytic function. No differences were observed in dendritic cell phenotypes (FIG. 12).

P210-PAM Immunization Reduced Atherosclerosis in ApoE^{-/-} Mice

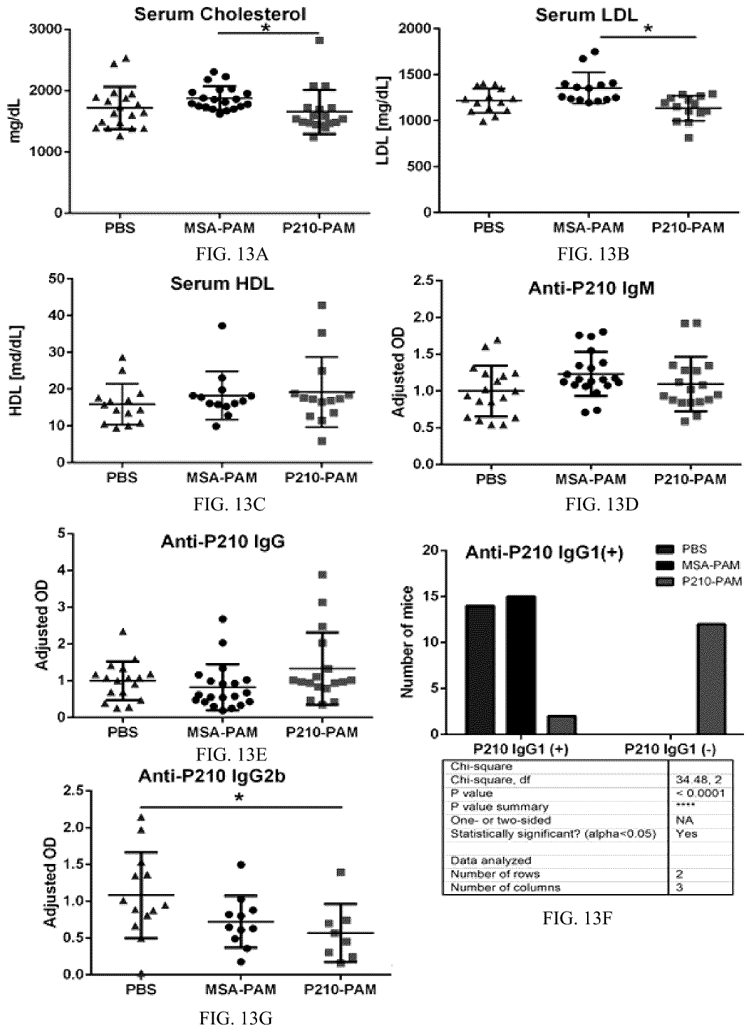

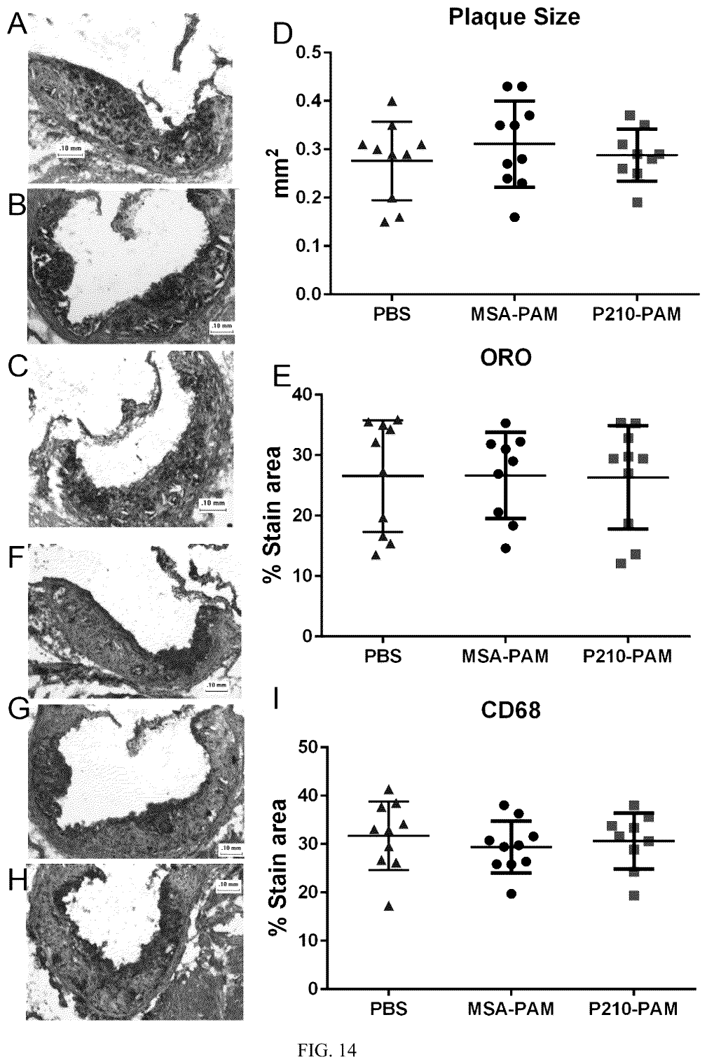

To test the effect of P210-PAM immunization on atherosclerosis, ApoE^{-/-} mice were subjected to the same immunization schedule described above and then fed high cholesterol diet from 13 weeks of age until euthanasia at 25 weeks of age. En face Oil-red-0 staining of the aorta (FIG. 5K) showed significantly reduced aortic atherosclerosis in P210-PAM immunized mice compared to PBS and MSA-PAM immunized mice (FIG. 5L). The mean circulating levels of total cholesterol or LDL-C in P210-PAM immunized mice were lower than those in MSA-PAM immunized mice but similar to the mean levels in PBS mice; whereas there was no difference in circulating level of HDL-C among three groups (FIG. 13A-13C). There was no difference in IgM or IgG level against P210 among groups but P210-PAM immunized group had reduced IgG1 and IgG2b against P210 (FIG. 13D-13G). No differences were observed in the aortic sinus plaque size, lipid stain, and macrophage content (FIG. 14).

P210-PAM Immunization Reduces IL-1R1 Expression and Modulates Macrophage Phenotype

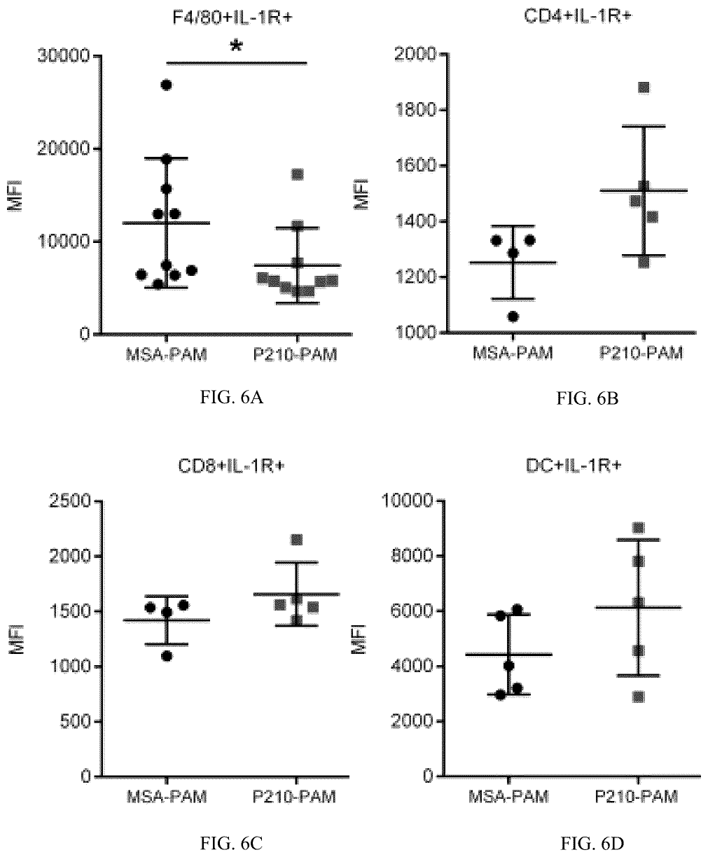

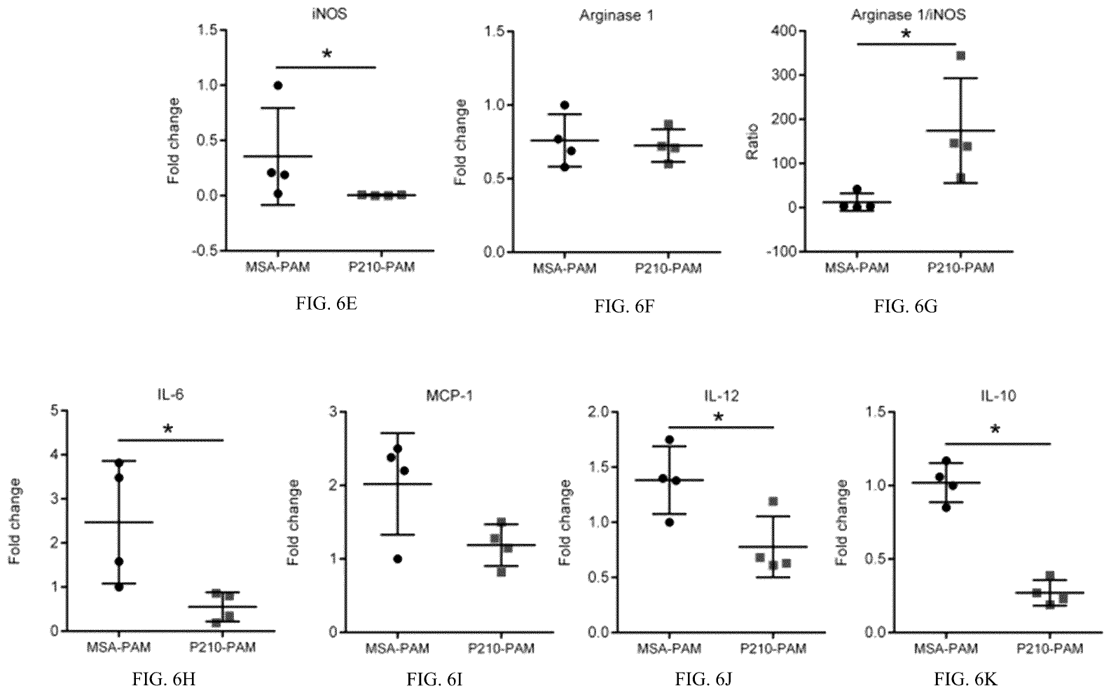

Since P210-PAM immunization elicited an antigen-specific regulation of CD4⁺ and CD8⁺ T cells, we next tested if such regulation involved the IL-1β signaling pathway given the known involvement of this pathway in atherosclerosis. There was a significant reduction in splenic IL-1R1, IL-6 and IL-17a gene expression in P210-PAM immunized mice but no difference in IL-1β gene expression when compared to MSA-PAM immunized mice (FIG. 5M-5P). Interestingly, the reduced IL-1R1 gene expression was primarily due to decreased expression on splenic F4/80⁺ cells, but not on CD4⁺, CD8⁺ T cells or DCs (FIG. 6A-6D), indicating modulation of macrophages by P210-PAM immunization. To delineate this pathway further, we examined the phenotypes of thioglycolate-induced peritoneal macrophages from P210-PAM or MSA-PAM-immunized mice. The mRNA expression of inducible NOS (iNOS), IL-6, IL-12 and IL-10 were all significantly reduced, with a trend toward decreased monocyte chemoattractant protein-1 (MCP-1), in macrophages from P210-PAM immunized mice (FIG. 6E, 6H-6K). Lack of difference in arginase 1 expression between the groups rendered higher arginase 1/iNOS expression ratio in macrophages from P210-PAM immunized mice (FIG. 6F, 6G).

ApOB_{KTTKQSFDL} (SEQ ID NO:2) Pentamer

The results thus far provided evidence that P210-PAM immunization provokes a response that modulates T cell function and macrophage phenotypes and reduces atherosclerosis in ApoE^{-/-} mice, supporting the feasibility of the immunogenic nanoparticle approach to reduce atherosclerosis. Antigen-based immune-modulation depends on the propensity of specific peptides to bind and be presented as immune-antigens by Class-I and Class II MHC. Our previous reports on P210 T cell responses in ApoE^{-/-} mice identified Class-I MHC/CD8⁺ T cell signaling as a mechanism for the protective effects of P210 immunization (Dimayuga, P. C., et al., J. Am. Heart Assoc. 2017, 6:doi: 10.1161/JAHA.116.005318; Chyu, K. Y., et al., PLoS. ONE. 2012, 7:e30780). An approach to bridging the experimental investigation towards translational application was therefore developed by screening Class-I HLA propensity to bind P210.

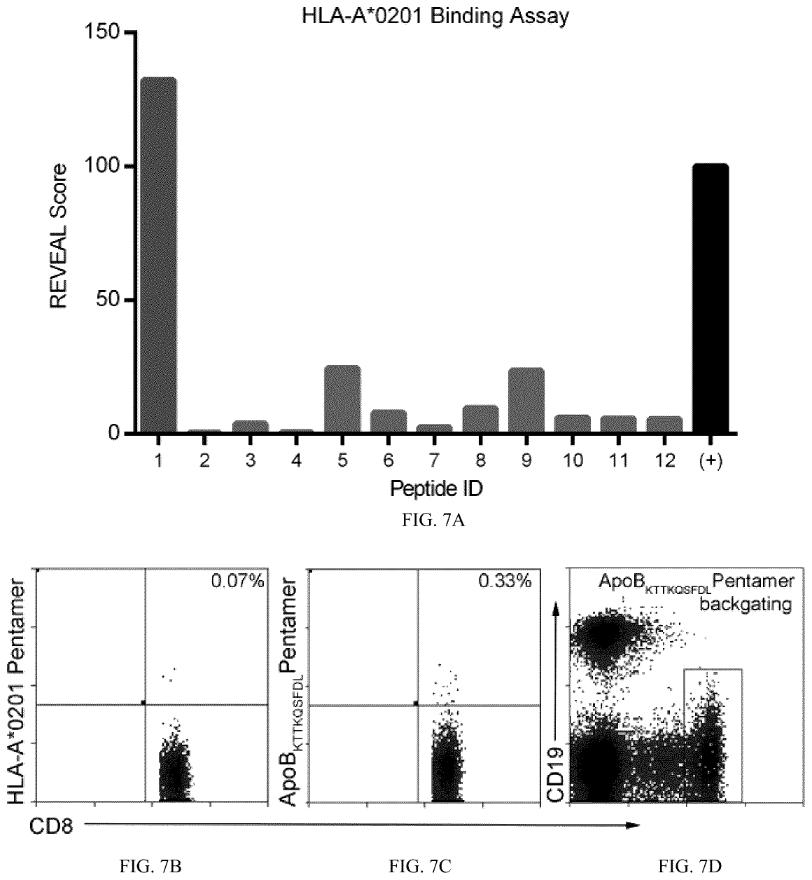

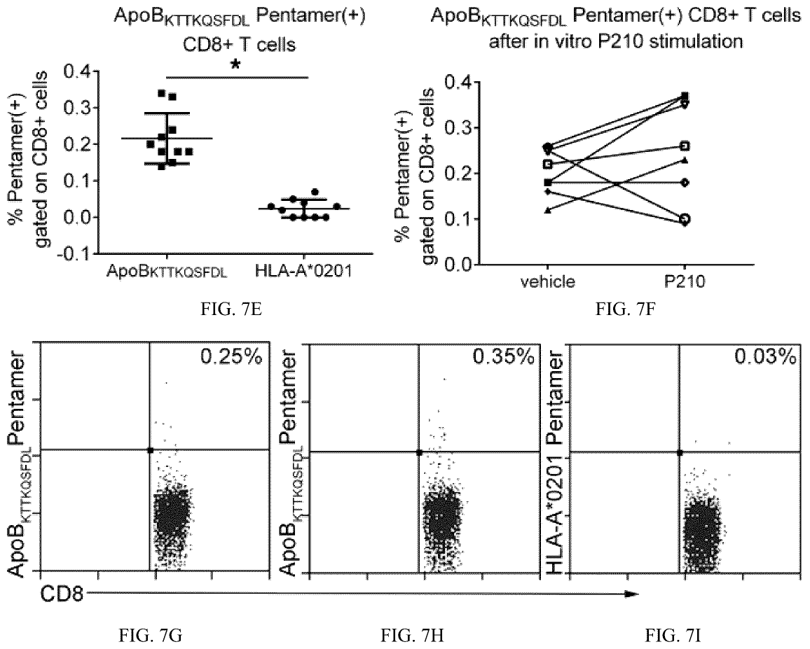

The human Class-I HLA that occurs with the highest frequency in North America is HLA-A*02:01; and P210 epitope binding to HLA-A*02:01 was tested by ProImmune using the REVEAL assay. The REVEAL assay used 9-mer sequential peptides of P210 to assess binding to HLA-A*02:01 (Table 2): a plurality of 9-amino acid fragments, each identical to residues 1-9, 2-10, 3-11, 4-12, 5-13, 6-14, 7-15, 8-16, 9-17, 10-18, 11-19, or 12-20 of P210. The first 9-mer scored well, comparable to the positive control (FIG. 7A), indicating that P210 contains at least one epitope that has the propensity to bind and potentially be presented by HLA-A*02:01, hence a pentamer based on this 9-mer sequence (ApOB_{KTTKQSFDL} (SEQ ID NO:2) pentamer) was generated for testing. ApOB_{KTTKQSFDL} (SEQ ID NO:2) pentamer was able to detect a small but significant population of P210-specific CD8+ T cells in PBMCs from healthy HLA-A*02:01(+) volunteers (FIG. 7B-7E). In 5 out of 8 tested samples, culturing these PBMCs with P210 for 5 days resulted in an increase of pentamer specific CD8⁺ T cells (FIG. 7F-7I).

A2Kb Transgenic ApoE^{-/-} Mice Express Functional Chimeric A2Kb Protein

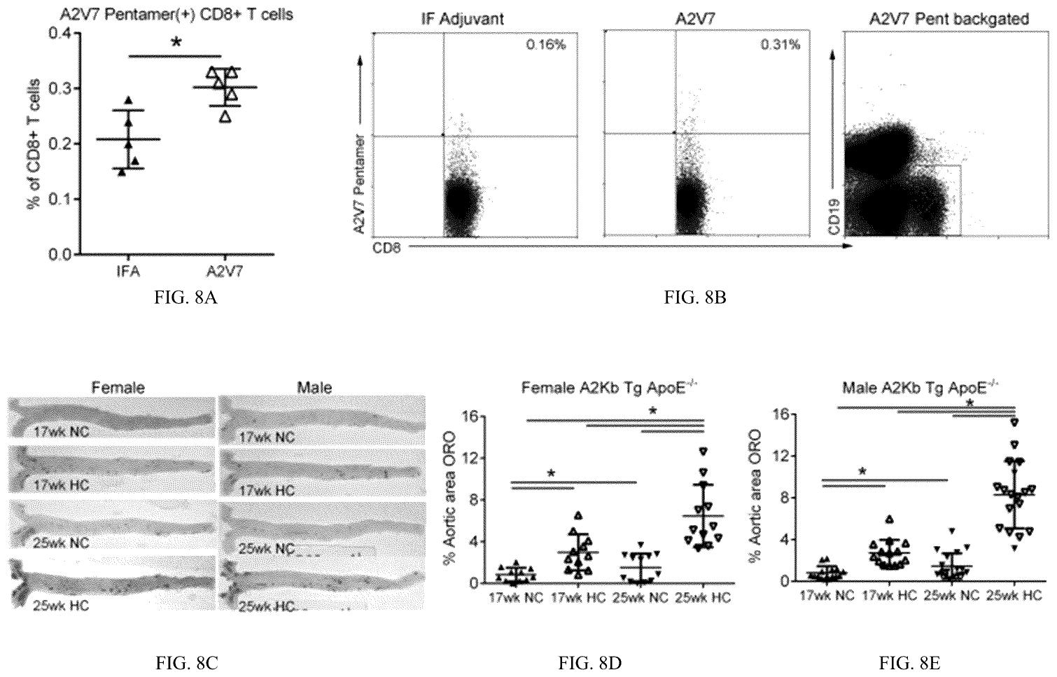

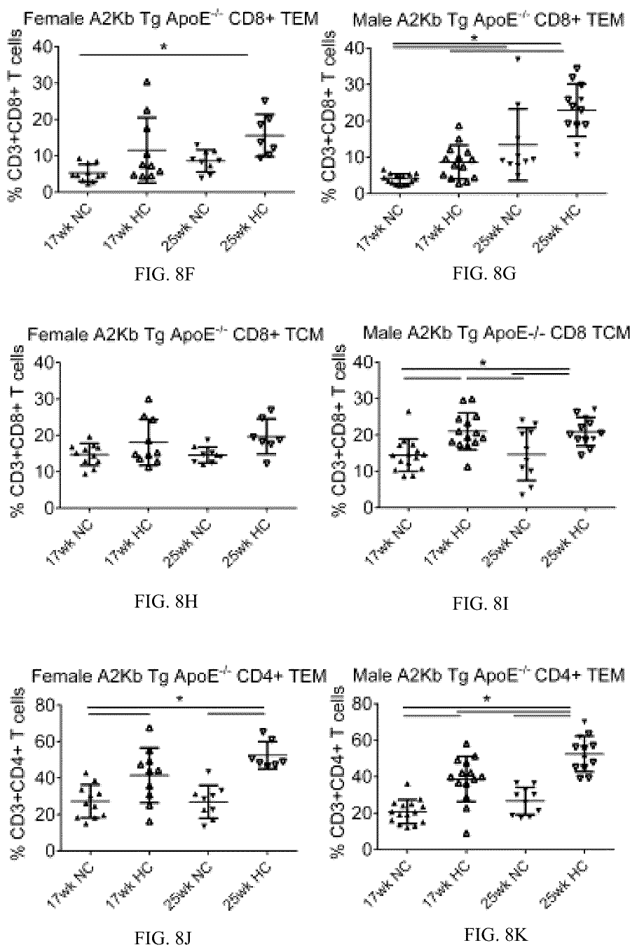

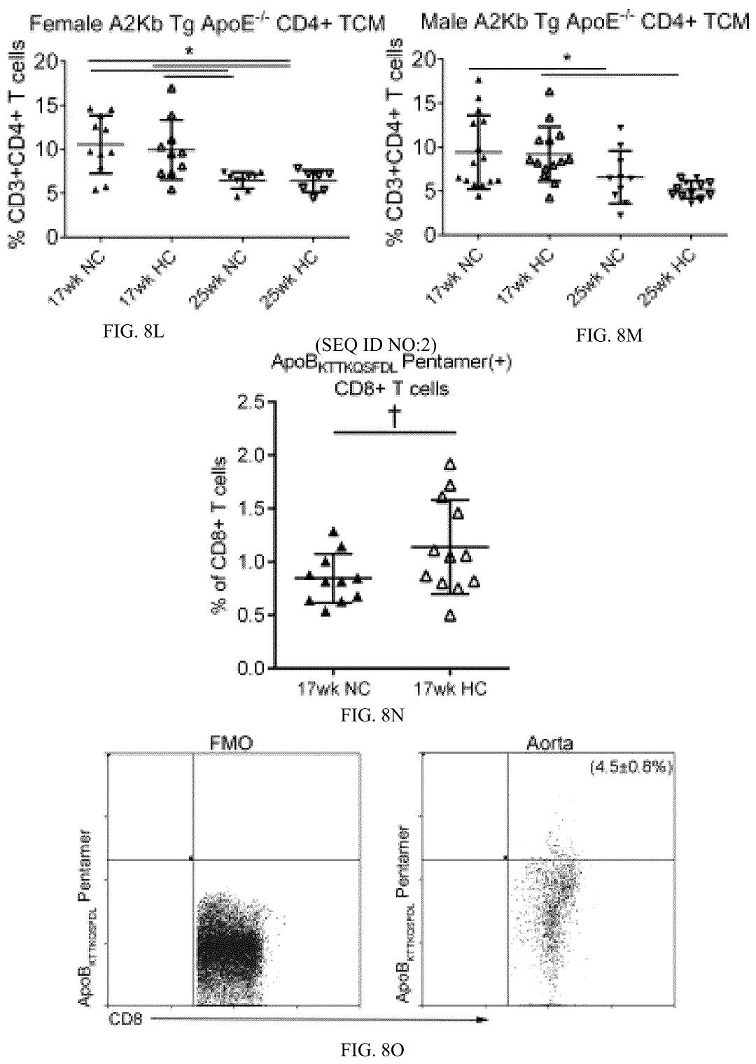

The transgene construct was synthesized for developing the mouse model. After obtaining A2Kb transgenic (Tg) ApoE^{-/-} offspring from breeding, immunization of male mice with an HLA A*02:01-restricted hepatitis C virus (HCV) peptide A2V7 significantly increased A2V7-pentamer⁺ CD8⁺ T cells in the spleen (P<0.05), compared to incomplete Freund's adjuvant (IFA)-injected male mice (FIG. 8A, 8B). The results demonstrated presentation of the HLA-A*02:01 restricted HCV peptide to activate CD8⁺ T cells supporting the functional expression of the chimeric transgene. Colony expansion was then undertaken to characterize atherosclerosis in the chimeric model.

High Cholesterol Diet Induces Atherosclerosis in A2Kb Tg ApoE^{-/-} Mice

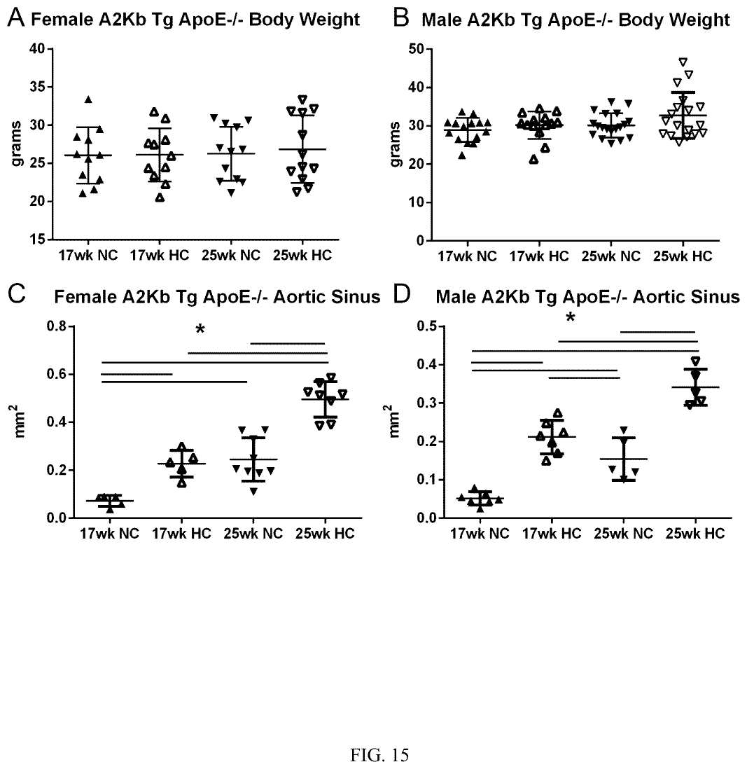

Feeding female A2Kb Tg ApoE^{-/-} mice with high cholesterol diet for 8 weeks starting at 9 weeks of age increased aortic atherosclerosis compared to normal chow feeding (FIG. 8C, 8D; 17 wk HC and 17 wk NC, respectively). High cholesterol diet for 16 weeks significantly increased circulating cholesterol levels (1274±297 mg/dL vs 661±119 mg/dL, P<0.001 by t-test) and aortic atherosclerosis (6.5±3.0% vs 1.5±1.3%, FIG. 8C, 8D; 25 wk HC and 25 wk NC, respectively) in female mice. Body weight was comparable in female mice fed with the two different diets (FIG. 15 panel A). Similarly, in male mice, high cholesterol diet feeding for 8 weeks compared to normal chow significantly increased aortic atherosclerosis (FIG. 8C, 8E).

High cholesterol diet for 16 weeks increased circulating cholesterol levels (1760±475 mg/dL vs 617±114 mg/dL, P<0.001 by t-test) and aortic atherosclerosis (8.3±3.2% vs 1.5±1.2%, FIG. 8C, 8E). Body weight was also comparable in male mice fed with the two different diets (FIG. 15 panel B). Aortic sinus lesion size was also significantly increased in mice fed with high cholesterol diet compared to those fed with normal chow (FIG. 15 panel C, panel D). The results show that aortic atherosclerosis burden is increased by high cholesterol diet in both male and female transgenic mice.

T Cell Profile and P210-Specific T Cells in A2Kb Tg ApoE^{-/-} Mice

Feeding A2Kb Tg ApoE^{-/-} mice with high cholesterol diet for 16 weeks significantly increased CD4⁺ effector memory (EM) T cells without change in central memory (CM) T cells in both female and male mice compared to normal chow feeding (FIG. 8F-8I). CD8⁺ EM T cells were also significantly increased in both high cholesterol diet-fed female and male mice. However, feeding high cholesterol diet increased CD8⁺ CM T cells significantly in male mice only (FIG. 8J-8M).

The results thus far showed that the A2Kb Tg ApoE^{-/-} mouse is a valid experimental model for atherosclerosis. Given that the results indicate responses are comparable between male and female mice, further analysis combined both sexes for the rest of the studies. ApoB_{KTTKQSFDL} (SEQ ID NO:2) pentamer staining showed that P210-specific CD8⁺ T cells were increased in A2Kb Tg ApoE^{-/-} mice fed with high cholesterol diet for 8 weeks compared to mice fed normal mouse diet (FIG. 8N, P=0.06). P210-specific CD8⁺ T cells were also observed in the aortic plaque of high cholesterol diet-fed mice by flow cytometric analysis of digested whole aortic tissue (FIG. 8O). These results support the potential involvement of P210-specific CD8⁺ T cells in atherosclerosis, in agreement with our previous studies, and use of the ApOB_{KTTKQSFDL} (SEQ ID NO:2) pentamer as a tool to assess P210-specific CD8⁺ T cell response in atherosclerosis.

P210-PAM Induced Persistent P210-Specific CD8⁺ T Cells in A2Kb Transgenic Mice and Reduced Atherosclerosis

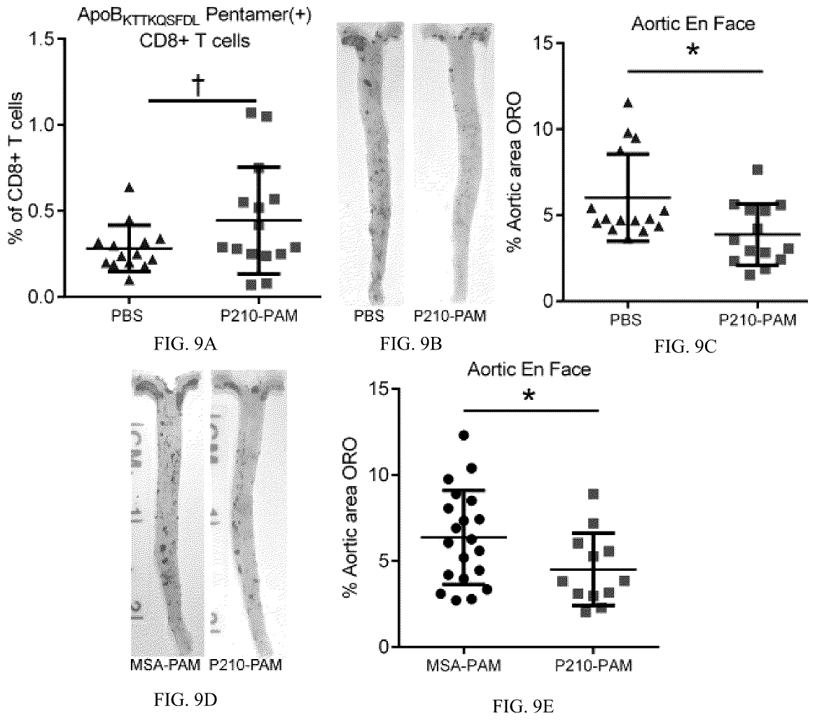

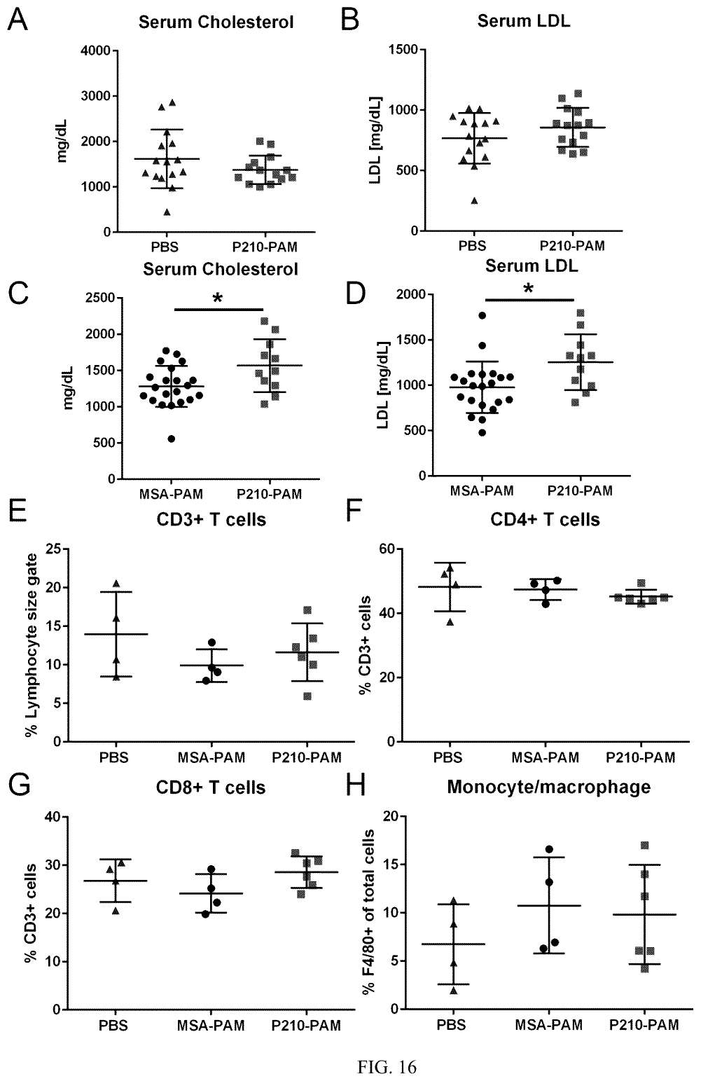

The results thus far show the A2Kb Tg ApoE^{-/-} mouse is a valid humanized atherosclerosis model to investigate translational use of P210-PAM as an antigen-specific immune-modulating therapy. A2Kb Tg ApoE^{-/-} mice were immunized as described and were fed with high cholesterol diet from 13 weeks of age until euthanasia at 25 weeks of age. We first tested if ApOB_{KTTKQSFDL} (SEQ ID NO:2) pentamer would detect P210-specific CD8⁺ T cells 13 weeks after the last booster injection. ApOB_{KTTKQSFDL} (SEQ ID NO:2) pentamer⁺ CD8⁺ T cells were detected in splenocytes of the immunized mice, trending higher compared to control mice injected with PBS (FIG. 9A, P=0.08). Furthermore, A2Kb Tg ApoE^{-/-} mice immunized with P210-PAM had significantly reduced aortic atherosclerosis compared to mice injected with PBS (FIGS. 9B & 9C). An additional group of A2Kb Tg ApoE^{-/-} mice were then immunized with MSA-PAM to determine if amphiphilic micelles with a different self-peptide would affect atherosclerosis in the humanized mouse model. There was no significant effect of MSA-PAM on atherosclerosis compared to PBS control, and P210-PAM immunized mice had significantly reduced atherosclerosis compared to MSA-PAM (FIG. 9D & 9E). There was no difference of circulating levels of total cholesterol or LDL-C between PBS and P210-PAM immunized mice (FIG. 16, panels A and B), whereas circulating levels of total cholesterol and LDL-C in P210-PAM immunized mice were higher than MSA-PAM immunized mice (FIG. 16, panels C and D). No differences were noted in T cell and macrophage infiltration of the aortas of the immunized A2Kb Tg ApoE^{-/-} mice (FIG. 16, panels E-H). The results supported P210-PAM as a viable translational immune-modulation therapy. The persistence of the P210-specific response can be assessed using a pentamer specific for an epitope of P210.

Overall in this study, we report the following new findings: (a) P210 specific T cell responses exist in human subjects with atherosclerotic cardiovascular disease (ASCVD); (b) P210 peptide can be taken up by dendritic cells via proteoglycan binding; (c) P210, when used in a nanoparticle platform (P210-PAM), co-stains with MHC-I and modulates T cells in ApoE^{-/-} mice; (d) In hypercholesterolemic ApoE^{-/-} mice, immunization with P210-PAM dampens P210-specific CD4+ T cell proliferative response and CD8+ T cell cytolytic response, modulates macrophage phenotypes, and significantly reduces aortic atherosclerosis; (e) We successfully developed and characterized a humanized atherosclerosis mouse model with HLA-A*02:01/Kb chimera in ApoE^{-/-} background, serving a translational bridge to potential future human testing; (f) Most importantly, immunization with P210-PAM in the chimeric mice reduced atherosclerosis, indicating P210-PAM is a viable strategy for potential human application. Although P210 has been shown by several investigators as an effective immune-modulation strategy to confer protective effect on atherosclerosis, our studies investigated its use in a nanoparticle formulation, and tested it on chimeric mice to demonstrate potential translational human application.

Investigations on the immune response against various ApoB-100 peptides, including P210, have demonstrated their potential use as peptide antigens for immune modulation therapies. Although P210 humoral immune response has been demonstrated in human ASCVD, information on cellular immune responses against P210 in humans is lacking. One hallmark feature of antigen-experienced T cells is activation upon antigen rechallenge. Given that patients with ACS have underlying atherosclerotic vascular disease, we tested if there is a population of P210-specific T cells that can be activated upon rechallenge of P210. The AIM assay showed induction of CD69⁺CD134⁺ activation markers on CD4⁺ T cells, supporting the existence of P210-experienced T cells in humans with atherosclerosis. Similarly, we found significantly different responses of CD8⁺ effector and effector memory T cells to P210 recall stimulation in PBMCs of patients with ACS when compared with the responses of CD8⁺ effector and effector memory T cells to P210 stimulation in PBMCs of healthy volunteers (FIG. 1I, 1J). Thus, our data support the notion that cellular immune responses to P210 exist in human ASCVD. Although the causal role of such CD8⁺ effector memory T cell response in ASCVD remains to be elucidated, it should be noted that memory T cells are enriched in atherosclerotic plaques, correlated with atherosclerosis in humans and mouse models, and associated with plaque progression and rupture. These observations highlight the involvement of memory T cells in atherosclerosis. To our knowledge, this is the first study to demonstrate P210-specific cellular immune responses in human ASCVD.

It is not clear how an auto-immune response to a self-antigen like P210 is triggered. However, the lysine-rich nature of the peptide may provide some insight. A common property of cell penetrating peptides (CPPs) is their cationic nature due to enrichment with lysine and/or arginine residues within the sequences. CPPs interact with negatively charged cell surface heparin sulfate proteoglycans to gain cell entry. Interestingly, part of the P210 peptide belongs to the proteoglycan binding domain of the ApoB-100 protein and has been shown to be a functioning CPP to generate antigen-specific CD8⁺ T cell response. Our results provided experimental evidence that P210 indeed has properties of a CPP with proteoglycan-binding properties that facilitates its internalization by DCs.

We have previously demonstrated the intrinsic CD8⁺ T cell recall response to P210 stimulation in naïve hypercholesterolemic mice (Dimayuga, P. C., et al., 2017, J Am. Heart Assoc. 6:doi: 10.1161/JAHA.116.005318). However, it is unknown if the immunologic property of P210 changes when formulated as PAM nanoparticles. We first demonstrated that DCs can uptake P210-PAM and P210 (or its fragment) costains with MHC-I using confocal microscopy. Our observation that P210-PAM immunization increased CD4⁺CD25⁺FoxP3⁺ and CD8⁺CTLA-4⁺ T cells indicated an induction of regulatory CD4⁺ and CD8⁺ T cells. This was further confirmed by functional experiments showing antigen specific reduction of CD4⁺ T cell proliferative response and CD8⁺ cytotoxic T cell response to P210. More importantly, P210-PAM immunization significantly reduced aortic atherosclerosis in mice when compared to control groups given phosphate buffered saline (PBS) (FIG. 9B, 9C).

A notable observation is that P210-PAM immunization, in addition to modulating T cells, also modulates macrophages. Interaction between T cells and monocytes/macrophages has been previously reported. CD8⁺ T cells promote bone marrow monocyte production via IFN-γ mediated mechanism in viral infection. Depletion of CD8⁺ T cells reduced atherosclerosis, decreased the number of mature monocytes in the bone marrow and spleen of hypercholesterolemic mice, reduced GM-CSF and IL-6 expression in bone marrow cells but did not affect the recruitment of monocytes to atherosclerotic plaques. In obese tissues, activated CD8⁺ T cells differentiated peripheral blood monocytes into macrophages. CD4⁺CD25⁺FoxP3⁺ T cells have been shown to induce alternatively activated monocytes with reduced inflammatory phenotype. Taken together, our data support the notion that P210-PAM elicits an interaction between T cells and macrophages and reduces the immune-inflammatory responses in atherosclerosis at the level of both innate and adaptive immunity.

The physicochemical properties of nanoparticles play a vital role in determining the immune responses of nanoparticle-based vaccines. Nanoparticles 20-200 nm in diameter are usually internalized by antigen presenting cells to elicit T cell response. Cationic nanoparticles with positive charges facilitate lysosomal escape and cross presentation to MHC-I. Solid core nanoparticles with antigen on the surface elicit stronger CD8⁺ T cell response whereas polymersomes with antigen incorporated inside the core bias toward CD4⁺ T cell response. This differential immune response based on physicochemical properties is not strictly dichotomous as reported data has shown solid core nanovaccines can also induce CD4⁺ T cell response. Our data indicate that cylindrical shaped P210-PAM elicits regulatory responses in both CD4⁺ and CD8⁺ T cells. Previous studies showed that the severity of autoantigen induced experimental autoimmune encephalomyelitis or type 1 diabetes could be reduced by delivering autoantigens via nanoparticles, by a mechanism by promoting differentiation of disease-primed autoreactive CD4⁺ T cells into TR1-like cells or by expanding memory-like antidiabetogenic CD8+ T cells (Clemente-Casares, X. et al., Nature 2016, 530:434-440; Tsai, S. et al., Immunity. 2010, 32:568-580). Given that P210 is potentially an atherogenic autoantigen, the induction of regulatory T cell responses by P210-PAM is consistent with this view. It should be noted that peptide loaded MHC-II or MHC-I complex was a part of nanoparticles used by the Clemente-Casares et al. and the Tsai et al. studies, whereas the P210-PAM in this study does not contain MHC molecules.

The mean reduction of atherosclerosis by P210-PAM immunization in the current study was 42% and 37% in ApoE^{-/-} mice and A2Kb-Tg ApoE^{-/-} mice, respectively. Although the reported athero-reduction effect from using various P210 formulation has been consistent across different studies, the reported immune responses to P210 differ. Some reported athero-reduction was associated with increased P210-related antibody production; some reported induction of regulatory T cell responses. Nevertheless, the reported data support the notion that P210 is capable of eliciting multiple humoral and cellular immune responses albeit each study used different dose, preparation and delivery method of P210.

A few studies have addressed the immune mediators for the athero-reduction effect produced by P210 immunization. Rattik et al. showed B cells pulsed with CTB-P210 (a fusion protein of P210 and the cholera toxin B subunit) reduced atherosclerosis after being transferred into naïve recipients in Vascul. Pharmacol. 2018, 111:54-61, but it is not clear if the B cells functioned as antigen-presenting cells or antibody-producing cells induced by peptide-pulsing. Another study showed that a P210 IgG antibody preparation from rabbits was able to reduce murine atherosclerosis in a passive immunization fashion. We previously reported P210 immunization was able to mount antibody response and a CD8 biased T cell response: using a cell transfer strategy, we demonstrated that CD8⁺ T cells, not B cells or CD4⁺CD25⁺ T cells, were the mediators responsible for the athero-protective effect of P210 immunization (Chyu, K. Y. et al., PLoS. ONE. 2012, 7:e30780).

The involvement of P210-specific CD8⁺ T cells described above prompted our investigation to transition towards translational studies. The first step to potentially translate our immunization strategy for clinical testing is to establish if this immunization strategy can elicit immune response in human subjects. To achieve this, it is necessary to develop tools and models to detect antigen specific T cells and for preclinical end-point testing, respectively. An HLA-A*02:01 based P210 related pentamer, named ApOB_{KTTKQSFDL} (SEQ ID NO:2) pentamer, was generated to track P210-specific CD8+ T cells as a marker for cellular immune response. Using this pentamer, we demonstrated the existence of a small but significant number of antigen specific CD8+ T cells that responded to P210 rechallenge in human PBMCs. We also generated an animal model with a prevalent human MHC-I allele, HLA-A*02:01, to produce proof-of-concept data before advancing this strategy to human testing. We chose HLA-A*0201 as a representative human MHC-I allele due to its high frequency in the population and generated a new animal model with transgenic expression of human HLA-A*02:01 in ApoE^{-/-} mouse on a C57BL/6J background. These mice mounted antigen specific CD8+ T cell response to the CD8 restricted peptide A2V7 from human hepatitis C virus as assessed by pentamer after immunization, indicating a functional HLA-A*02:01 allele. With P210-PAM immunization, these mice elicited higher splenic HLA-ApOB_{KTTKQSFDL} (SEQ ID NO:2) pentamer(+) CD8+ T cells when compared to non-immunized mice. P210-PAM immunization significantly reduced aortic atherosclerosis when compared to control groups, supporting the potential use of P210-PAM for human testing. Given the same genetic background between ApoE^{-/-} mouse and chimeric mouse, we speculate P210-PAM immunization modulates macrophages, CD4+ and CD8+ T cells in A2Kb-Tg ApoE^{-/-} mice similarly to ApoE^{-/-} mouse. However, this remains to be confirmed.

The concept of using active immunization strategies to reduce atherosclerosis has progressed in the past three decades. The search for suitable antigens has evolved from using the whole LDL molecule as an antigen to subunits of lipoprotein such as ApoB-100 peptides. In murine atherosclerosis, immune responses to LDL or its related ApoB-100 peptides are present, and modulation of such responses by active immunization with LDL or ApoB-100 peptides confers athero-protective effects. If the same analogy applies to humans, given the existence of immune responses to LDL or ApoB-100 peptides in humans, we hypothesize similar athero-protective effect from active immunization in humans. Here we demonstrate physicochemical and immunological properties of P210-PAM and its effects on T cell responses and atherosclerosis, supporting the use of P210-PAM as an immune-modulation strategy targeting atherosclerosis. Such nanoparticle platforms are suitable for human application. More importantly, our successful use of P210-PAM in chimeric mice with human MHC-I allele provided proof-of-concept data showing potential efficacy in human immune system and paves the way for future testing in humans.

### Example 2. Materials and Techniques

TABLE 1
Characteristics of human subjects.

| | Control | ACS |
|---|---|---|
| | (N = 14) | (N = 13) |
| Mean age | 58.2 ± 10.4 | 58.1 ± 14.6 |
| Male sex | 71% | 77% |
| Female sex | 29% | 23% |
| Mean LDL cholesterol (mg/dL) | N/A | 109.6 ± 40.3 |
| Use of cholesterol lowering medication* | N/A | 46% |

ACS: acute coronary syndrome;
N/A: not available;
*determined at time of admission, 1 patient was on Praluent.

TABLE 2
P210 epitope analysis.

| Peptide ID | Sequence | SEQ ID |
|---|---|---|
| 1 | KTTKQSFDL* | SEQ ID NO: 2 |
| 2 | TTKQSFDLS | SEQ ID NO: 3 |
| 3 | TKQSFDLSV | SEQ ID NO: 4 |
| 4 | KQSFDLSVK | SEQ ID NO: 5 |
| 5 | QSFDLSVKA | SEQ ID NO: 6 |
| 6 | SFDLSVKAQ | SEQ ID NO: 7 |
| 7 | FDLSVKAQY | SEQ ID NO: 8 |
| 8 | DLSVKAQYK | SEQ ID NO: 9 |
| 9 | LSVKAQYKK | SEQ ID NO: 10 |
| 10 | SVKAQYKKN | SEQ ID NO: 11 |
| 11 | VKAQYKKNK | SEQ ID NO: 12 |
| 12 | KAQYKKNKH | SEQ ID NO: 13 |

Sequential 9-mer P210 peptides analyzed for binding to HLA-A*02:01.
*High binding score as depicted in corresponding REVEAL binding assay result in FIG. 7A.

Human PBMC

The protocols were approved by the Cedars-Sinai Institutional Review Board (IRB). Peripheral blood mononuclear cells (PBMCs) were isolated from blood collected from 13 patients with ACS within 72 hours of admission to the Cedars-Sinai Cardiac Intensive Care Unit. Patients were consented under the approved IRB protocol Pro48880. Exclusions were inability to give informed consent, age less than 18 years old, active cancer treated with chemotherapy or radiation, patients taking immune-suppressive drugs, and pregnant women. The data collected was limited to age, sex, LDL levels, and use/non-use of cholesterol-lowering medication. PBMCs were isolated using Ficoll density gradient centrifugation and cryo-preserved in commercially available cryogenic solution (Immunospot) in liquid nitrogen. Cryo-preserved PBMCs from healthy controls (N=14) were purchased from a commercial source (Immunospot).

Activation Induced Marker Assay (AIM Assay) in Human PBMC

Cryo-preserved PBMCs were thawed, rinsed in anti-aggregation solution (Immunospot), and seeded in culture plates at a density of 3×10⁶ cells per ml of RPMI 1640 medium supplemented with 10% heat-inactivated pooled human serum and 1× antibiotic/antimycotic. After resting for 4 hours, cells were preincubated with 0.5 mg/ml anti-CD40 antibody for 15 minutes then stimulated with 20 μg/ml P210 peptide, 0.5× T cell stimulation cocktail containing PMA and ionomycin (Thermo Fisher), or CMV (pp65) Peptide Pool (StemCell Tech) as a non-relevant antigen control, whereas cells without treatment served as non-stimulated control. Cells were harvested 16 hours after seeding, stained for viability (LIVE/DEAD Fixable Aqua Dead Stain Kit, Thermo Fisher), and subjected to cell surface staining for flow cytometry using the following antibodies: CD4, CD8, CD25, CD69, OX40 (CD134), CD137 (4-1 BB) and CD154 (CD40L). Isotypes were used as staining control and eFluor506 labelled CD14, CD16 and CD19 antibodies were used as dump staining to exclude B cells, dendritic cells, macrophages, granulocytes, eosinophil cells and neutrophil cells. The results are expressed as fold change (ratio between the signal in the antigen stimulated condition and the signal in the unstimulated condition) for each subject, consistent with the reported AIM assay. Antibodies used in AIM assay are listed in Table 3.

TABLE 3
Exemplary antibodies used in AIM assay.

| Marker | Fluorochrome | Clone |
|---|---|---|
| CD4 | BUV395 | SK3 |
| CD8a | eFluor450 | RPA-T8 |
| CD25 | FITC | M-A251 |
| CD69 | PE | FN50 |
| CD134 (OX40) | APC | ACT35 |
| CD137 | APC-eFluor 780 | 4B4 (4B4-1) |
| CD154 (CD40L) | BV711 | 24-31 |
| CD14 | eFluor506 | 61D3 |
| CD16 | eFluor506 | eBioCB16 |
| CD19 | eFluor506 | HIB19 |

Peptide Stimulation of Human PBMC

Cryo-preserved PBMCs were thawed, rinsed and cultured as in AIM assay but without resting. Cells were stimulated with 20 μg/ml P210 peptide or 0.5× T cell stimulation cocktail containing phorbol 12-myristate 13-acetate (PMA) and ionomycin (Thermo Fisher) with non-treated cells serving as negative control. Culture medium was added at ⅓ of the starting volume 48 hours later to replenish the nutrients in the medium. Cells were harvested 72 hours after seeding, stained for viability (LIVE/DEAD Fixable Aqua Dead Stain Kit, ThermoFisher), and subjected to cell surface staining for flow cytometry using the following antibodies: CD3, CD4, CD8, CD45RA, CD45RO, CD62L, and CD197 (CCR7). Isotypes were used as staining control. CD4⁺ or CD8⁺ T Effector cells were gated on CD45RO⁺CD62L⁻CD197⁻. T Effector Memory cells were CD45RO⁺CD45RA⁻CD62L⁻CD197⁻. Antibodies used are listed in Table 4.

TABLE 4
Exemplary antibodies used for human effector memory T cells.

| | Fluorochrome | Clone |
|---|---|---|
| Viability | Aqua Blue | |
| CD3 | eF450 | UCHT1 |
| CD4 | BUV395 | SK3 |
| CD8a | APC | SK1 |
| CD45RA | FITC | L48 |
| CD45RO | APC-eF780 | UCHL1 |
| CD62L | BV711 | DREG-56 |
| CD197 | PE | 150503 |

Results were Tabulated as Response Index Using the Following Calculation:

(% peptide stimulation−% no stimulation)/(% cocktail stimulation)×100.

The results are expressed as Response Index to account for inherent variations introduced by culturing cells in vitro over time, controlled for by assessing response relative to baseline cell phenotype (% no stimulation) and maximal stimulation (% cocktail stimulation) for each subject PBMC. Each data point represents one subject.

Animals

All mice were maintained under standard animal housing conditions with a 12-h light-dark cycle and were fed ad libitum with a regular chow diet (5015, PMI Nutrition International, USA) unless mentioned otherwise. All animal procedures were done in compliance with National Institutes of Health guidelines and were approved by IACUC. B6.129P2-ApoetmlUnc/J (ApoE−/−) mice were purchased from Jackson Lab (Stock No: 002052, Bar Harbor, Me). A2Kb transgenic CB6F1-Tg(HLA-A*02:01/H2-Kb)A*0201 mice were purchased from Taconic Biosciences (Model 9659).

Amphiphile Synthesis, Assembly and Characterization

Amphiphile synthesis: Peptide amphiphiles were synthesized by conjugating peptides to the 1′-3′-dihexadecyl N-succinyl-L-glutamate (diC16) hydrophobic tail (Joo J et al. Molecules. 2018; 23:2786). DiC16 was synthesized by first mixing hexadecanol (22.4 g, 0.092 mol), L-glutamic acid (6.8 g, 0.047 mol), and para-toluenesulfonic acid (10.5 g, 0.051 mol) to yield 1 ‘-3’-dihexadecyl L-glutamate, which was then purified through Buchner funnel filtration through acetone and identified through 1H-NMR. This was then mixed with succinic anhydride in 1:1 tetrahydrofuran:chloroform to yield 1′-3′-dihexadecyl N-succinyl-L-glutamate (diC16). The crude diC16 was then crystallized overnight at 4° C., purified through Buchner funnel filtration through diethyl ether, and identified via 1H-NMR.

One mmol of P210 or mouse serum albumin (MSA; QTALAELVKHKPKATAEQLK (SEQ ID NO:47)) peptides were synthesized on an automated peptide synthesizer (PS3, Protein Technologies, Tucson, AZ, USA) with Fmoc-mediated solid phase peptide synthesis. Then peptides were conjugated to 1 mmol diC₁₆ overnight through a peptide bond using N,N-diisopropylethylamine (1.25 mmol) and O-benzotriazole-N,N,N′,N′-tetramethyl-uronium-hexafluoro-phosphate (1.125 mmol). Peptide amphiphiles were then cleaved from the solid phase resin by shaking in a 95:2.5:2.5% volume trifluoroacetic acid:triisopropylsilane:water solution for 2 hours, precipitated in ice-cold diethyl ether, and lyophilized. Peptide amphiphiles (PA) were purified using reverse-phase, high-pressure liquid chromatography (RP-HPLC, Prominence, Shimadzu, Columbia, MD, USA) on a Luna C4 column (Phenomenex, Torrance, CA, USA) at 55° C. with 0.1% formic acid in water and acetonitrile mixtures as mobile phases. The purity of eluted peptide amphiphiles was characterized using matrix-assisted laser desorption/ionization time-of-flight mass spectroscopy (MALDI-TOF-MS, Bruker, MA, USA). As shown in FIG. 11 panel A, the expected mass peak for the P210 PA is 3058 g/mol, and as shown in FIG. 11 panel B, the expected mass peak for the MSA PA is 2882 g/mol. Fluorescently labeled diC16-cy7 amphiphiles were synthesized by reacting diC16 with cy7-amine, 1-ethyl-3-(3-dimethylaminopropyl)-carbodiimide (EDC), N-hydroxysuccinimide (NETS), and triethanolamine (TEA) in dimethyl sulfoxide (DMSO) at a 1:1.5:4:1:1 ratio diC16:cy7-amine:EDC:NHS:TEA. The EDC, NETS, and TEA were divided into five aliquots, with the first four aliquots added sequentially 2 h after the previous aliquot, while the fifth aliquot was added 12 h after the fourth aliquot. Afterwards, the reaction was stirred for an additional 24 h before purification through RP-HPLC. The expected mass peak for the diC16-cy7 is 1326 g/mol.

Micelle assembly: Micelles were prepared through thin-film hydration as previously reported (Joo J et al. Molecules. 2018; 23:2786). Briefly, peptide amphiphiles were dissolved and sonicated in methanol, before evaporation under a nitrogen stream into thin films. Films were hydrated in water or PBS, sonicated and heated to 80° C. for 30 minutes before cooling to room temperature. Fluorescently labeled P210 or MSA PAMS were synthesized by mixing P210 or MSA PAs with diC16-cy7 at a 90:10 molar ratio.

Micelle characterization: The shape and morphology of micelles were characterized through transmission electron microscopy (T_{EM}). Seven μL of 100 μM P210 PAMs was placed onto 400 mesh carbon grids (Ted Pella, Redding, CA, USA) for 5 minutes, before excess liquid was wicked, and the grids were washed with water. The grids were then stained 2% uranyl acetate, washed again with water, and dried before imaging on a JEOL JEM 2100-F T_{EM} (JEOL, Tokyo, Japan). Micelle size, polydispersity, and zeta potential were characterized using a Dynapro Nanostar system (Wyatt, Santa Barbara, CA, USA). One hundred μM of micelles were suspended in water and placed in a quartz cuvette with a platinum dip probe (n=3) for size, polydispersity, and zeta potential analysis.

Dendritic Cell Uptake of FITC-Labeled P210 and P210-PAM

P210 peptide (Euro-Diagnostica AB, Sweden; KTTKQSFDLSVKAQYKKNKH (SEQ ID NO:1)) was labeled with FITC using a commercially available kit (Thermo Fisher). To prepare FITC-P210-PAM, P210 peptide was first labeled with FITC on the last lysine on C-terminal when the peptide was synthesized, then the labelled P210-FITC were assembled to FITC-P210-PAM using methods described above for micelle assembly.

Bone marrow derived dendritic cells (BMDCs) were prepared using BM cells from femurs and tibiae of male ApoE^{-/-} mice. After depletion of erythrocytes with lysis buffer, BM cells were cultured in 10 cm dishes with 10 ml complete RPMI-1640 medium supplemented with 20 ng/ml GM-CSF and 10 ng/ml IL-4. On Day 2, 10 ml fresh culture medium was added to each dish, then 10 ml medium was replaced with fresh medium on day 4 and 6. On day 8, non-adherent immature dendritic cells were harvested into new culture medium containing 100 μg/ml P210-FITC or FITC-P210-PAM in complete RPMI-1640 medium. After a 4 h incubation for P210-FITC or 6 h incubation for FITC-P210-PAM, cells were collected and stained with antibodies to CD11c (N418, Invitrogen) or CD11c and H2-Kb (AF6-88.5.5.3, Invitrogen), respectively. Cells were washed and fixed in 4% paraformaldehyde followed by washing and staining with the fluorescent nuclear stain Hoechst 33342 (Thermofisher) or DAPI (Invitrogen). Washed cells were then smeared on a slide, briefly air-dried in the dark, and fixed in cold acetone. Photographs were then taken on a Leica or Zeiss confocal microscope visualized with liquid fluorescent mounting medium. Untreated DC were collected and smeared on slides, air dried, then stained with Giemsa staining reagent (Beckman Coulter) according to the kit instruction with photos taken using light microscope to demonstrate dendrites.

For flow cytometric experiments, P210-FITC uptake was assessed after a 2 h incubation and staining for CD11c. For heparin binding experiments, 100 μg/mL P210-FITC was pre-incubated with 100 U/mL heparin for 30 minutes at room temperature and centrifuged at 1000×g for 5 min. The supernatant was carefully removed and added to the cell culture. Cells were collected after 2 h and stained for CD11c for flow cytometry. In a separate experiment, DCs were treated with p-nitrophenyl-β-D-xylopyranoside (pNP-xyl), a competitive inhibitor of heparan sulfate chain addition, for 18 hours at a final concentration of 3 mM. DCs were then incubated with P210-FITC for 2 hours, collected, and stained with anti-CD11c (N418) for flow cytometry.

PAM Biodistribution In Vivo

The in vivo biodistribution of P210-PAM or MSA-PAM was evaluated by injecting 1 mM cy7-labeled PAMs in 100 μl volume subcutaneously into the scruff of the neck in C57BL/6J mice (n=4). After injection, mice were shaved and imaged over 7 days (168 h) using an AMI HTX imaging system (Spectral Instruments Tucson, AZ, USA). A separate group of mice was euthanized 48 hrs after injection to harvest injection site for immunostaining.

T Cell Immune Response to P210-PAM in Naïve Hypercholesterolemic Mice

Splenocytes were collected from 25 week-old ApoE^{-/-} mice euthanized after 16 weeks of high cholesterol diet feeding consisting of 0.15% cholesterol, 21% fat (TD.88137, Envigo). RBC lysed splenocytes were incubated with 20 μg/ml P210-PAM in complete RPMI-1640 medium for 48 h then stained with CD3e (145-2C11), CD4 (GK1.5), CD8b (H35-17.2, eBioscience), CD44 (IM7) and CD62L (MEL-I4) antibodies for T effector/memory cell profiling using flow cytometry.

Immunization with P210-PAM and Phenotyping Atherosclerotic Lesions

Seven week-old ApoE^{-/-} mice fed normal chow received a subcutaneous injection of one of the following: P210-PAM, MSA-PAM, or PBS. PAM dose used was 100 μg/mouse. Booster injections were administered at 10 and 12 weeks of age. Some mice were euthanized one week after the second booster for immune profiling. The rest of the mice were fed high cholesterol diet for 12 weeks and euthanized at 25 weeks of age. Whole aortas were cleaned, processed and stained with Oil-red-0 to assess the extent of atherosclerosis en face. Frozen heart bases embedded in OCT (Optimum Cutting Temperature, Tissue-Tek) were cryo-sectioned starting from the appearance of 3 complete aortic valves. Three slides with 2 sections on each slide at 4-5 slides intervals were grouped for aortic sinus histomorphometry. Plaque sizes and lipid content were accessed by Oil-Red-O staining using standard protocol. Macrophage in atherosclerotic lesions in the aortic sinus was assessed by immunohistochemistry staining with anti-CD68 (FA-11, BioLegend) antibody, following with incubation with appropriate secondary antibody using standard protocol. Computer-assisted morphometric analysis was performed by a blinded observer using ImagePro (ImagePro Plus, version 4.0, Media Cybernetics Inc., Rockville, Maryland). Serum levels of total cholesterol, LDL-C and HDL-C were measured using commercially available kits according to manufacturer's instruction (Wako).

ELISA for P210 Antibodies

Flat-bottomed 96-well polystyrene plates (MaxiSorp, Germany) were pre-coated with 100 μl P210 (20 μg/ml) in Na₂CO₃—NaHCO₃ buffer (pH9.6) overnight at 4° C. to assess antibody levels using standard protocol. The coating concentration and serum dilution was optimized in pilot experiments. Goat anti-mouse HRP-IgG (Pierce), IgM (Southern Biotech), rat anti mouse-IgG1-HRP (Invitrogen) and goat anti mouse-IgG2b-HRP (Southern Biotech) were used as detecting antibodies and the bound antibodies were detected by developing in ABTS (Southern Biotech) as substrate and optical density values were recorded at 405 nm. Given there is no purified P210 antibody that can be used for standardization, OD of individual mouse in each group was normalized against the mean OD from PBS group and presented as “adjusted O.D.” in the figures.

Immune Profile of P210-PAM Immunized Mice

Splenocytes of immunized ApoE^{-/-} mice that were euthanized at 13 weeks of age (1 week after second booster) were subjected to RBC lysis. An aliquot of splenocytes were stained for CD4 (GK1.5, BD Bioscience), CD8 (YTS156.7.7, BioLegend), CD25 (PC61.5, eBioscience), CTLA-4 (UC10-4B9, BioLegend), FoxP3 (R16-715, BD Bioscience), and PD-1 (29F 1A12, BioLegend) and analyzed by flow cytometry excluding non-viable cells. A second aliquot was used to assess cytolytic activity using CD107a (1D4B) staining. Briefly, splenocytes were incubated in complete RPMI-1640 medium with 2.5 μg/ml fluorescent CD107a antibody and 5 μg/ml P210 for 1 h.

Monensin (lx) was added and the cells incubated for another 4 hours. Cells were then collected and stained with fluorescent CD3e (145-2C11, BD Pharmingen) and CD8b (H35-17.2, Invitrogen) antibodies. The cells were analyzed by flow cytometer excluding non-viable cells. T cell proliferation was assessed using BrdU. Briefly, splenocytes were cultured in complete RPMI-1640 medium at 2.5×10⁶ cells/ml and stimulated with P210 (20 μg/ml). Cells stimulated with Concanavalin A (2.5 μg/ml) served as positive control. Untreated cells served as baseline controls. After 48 h, BrdU was added at a final concentration of 10 μM. Cells were collected after 24 h and stained for CD3e (BM10-37, BD Bioscience), CD4 (GK1.5, BD Bioscience), CD8b (H35-17.2, Invitrogen) and BrdU (3D4, BD Pharmingen) according to manufacturer's instructions (BrdU Flow Kit, BD Pharmingen) then analyzed by flow cytometry. Proliferation index was calculated as [(% BRDU⁺ cells in P210 peptide stimulation−% BRDU⁺ cells in no stimulation)/(% BRDU⁺ cells in Con A stimulation)]×100.

Induction of Peritoneal Macrophages

Seven weeks old ApoE^{-/-} mice fed normal chow were immunized as previously described. At 13 weeks of age (1 week after second booster), mice received peritoneal injection of 1 ml 3% thioglycollate medium (in PBS) and cells from peritoneal cavity were harvested 72 hrs after injection. Cells were seeded to culture dish and incubated at 37° C. for 4 hrs to obtain attached peritoneal macrophages.

qPCR

Total RNA was extracted from spleens or peritoneal macrophages enriched from peritoneal exudate by pre-attaching to culture plates using TRIzol (Thermo Fisher). cDNA synthesis and quantitative real-time PCR were then performed using SuperScript VILO cDNA Synthesis Kit (Thermo Fisher), and iTaq Universal SYBR Green Supermix and iQ5 Real-Time PCR Detection System (Bio-Rad), respectively, per manufacturers' protocols. GAPDH served as the reference gene and results were expressed as fold-change relative to non-treated cells of each sample using the CtΔΔ method. Primer sequences used for qPCR are listed in Table 5.

TABLE 5
Primers used in qPCR.

| Gene | Forward | Reverse |
|---|---|---|
| GAPDH | atcactgccacccagaa | cacattgggggtagga |
| | gac | acac |
| | (SEQ ID NO: 14) | (SEQ ID NO: 15) |
| arginase 1 | ggcagaggtccagaaga | gccagagatgcttgga |
| | atg | actg |
| | (SEQ ID NO: 16) | (SEQ ID NO: 17) |
| iNOS | agtggtccaacctgcag | ctgatgttgccattgt |
| | gtc | tggt |
| | (SEQ ID NO: 18) | (SEQ ID NO: 19) |
| MCP-1 | cagccagatgcagttaa | gcctactcattgggat |
| | cgc | catcttg |
| | (SEQ ID NO: 20) | (SEQ ID NO: 21) |
| IL-6 | ctgcaagagacttccat | agtggtatagacaggt |
| | ccag | ctgttgg |
| | (SEQ ID NO: 22) | (SEQ ID NO: 23) |
| IL-10 | tttgaattccctgggtg | acaggggagaaatcga |
| | agaa | tgaca |
| | (SEQ ID NO: 24) | (SEQ ID NO: 25) |
| IL-12 | cacgctacctcctcttt | cagcagtgcaggaata |
| | ttg | atgtt |
| | (SEQ ID NO: 26) | (SEQ ID NO: 27) |
| IL-1ß | gggcctcaaaggaaaga | ttgcttgggatccaca |
| | atc | ctct |
| | (SEQ ID NO: 28) | (SEQ ID NO: 29) |
| IL-1R1 | caggagaagtcgcagga | tggaacagagccagtg |
| | agt | tcag |
| | (SEQ ID NO: 30) | (SEQ ID NO: 31) |
| IL-17a | tctctgatgctgttgct | cgtggaacggttgagg |
| | gct | tagt |
| | (SEQ ID NO: 32) | (SEQ ID NO: 33) |

Detection of ApOB_{KTTKQSFDL} (SEQ ID NO:2) Pentamer (+) CD8+ T Cells in Human PBMCs

Proimmune was contracted to screen for potential binding epitopes in P210 to HLA-A*02:01. First 9-mer sequence in P210 was found to have high binding score and an HLA-A*02:01 pentamer based on this 9-mer sequence, named ApOB_{KTTKQSFDL} (SEQ ID NO:2) pentamer, was then purchased from Proimmune. For pentamer staining, commercially available HLA-A*02:01 typed cryo-preserved PBMCs (Immunospot) were thawed, rinsed in anti-aggregation solution (Immunospot) and divided into 2×10⁶ cell aliquots. ApOB_{KTTKQSFDL} (SEQ ID NO:2) pentamer staining was performed according to manufacturer's instruction, with the HLA-A*02:01 Negative Pentamer (ProImmune) as negative control. Each sample stained for ApOB_{KTTKQSFDL} (SEQ ID NO:2) pentamer had its corresponding negative control stain. Cells were washed and then stained for CD8 (LT8) andddCD19 (HIB19). Cells were again washed after staining and resuspended in 1% paraformaldehyde in 1% BSA/0.1% sodium azide and analyzed. ApOB_{KTTKQSFDL} (SEQ ID NO:2) pentamer positive cells for each sample were determined based on the corresponding Negative Pentamer.

A2Kb Transgenic ApoE^{-/-} Mice

A2Kb transgenic ApoE^{-/-} (A2Kb Tg ApoE^{-/-}) mice were generated as briefly described: A 3867 bp full-length chimeric A2Kb gene was cloned into pCR-XL-TOPO T vector (Thermo Fisher) and the amplified recombinant plasmids containing A2Kb gene were digested with restriction enzymes to yield ˜3.9-kb fragments containing the chimeric A2Kb gene for fertilized ApoE^{-/-} eggs microinjection by the Cedars Sinai Rodent Genetics Core. Germline-transmitted A2Kb chimeras obtained were screened by PCRs detecting HLA A*02:01 fragments and flow cytometric analysis of A2Kb protein expression on the surface of peripheral blood mononuclear cells (PBMCs).

A transgenic ApoE^{-/-} male mouse was identified and crossbred with female ApoE^{-/-} mice. The A2Kb transgenic offspring selected by flow cytometric analysis of chimeric A2Kb protein expression on peripheral blood cells were used for further breeding or experiments.

Functional Expression of A2Kb Transgene

Male A2Kb Tg ApoE^{-/-} mice were immunized with the HLA-A*02:01-restricted peptide A2V7 from human hepatitis C virus (HCV NS5a 1987-1995, VLSDFKTWL (SEQ ID NO:34); ProImmune) emulsified in incomplete Freund's adjuvant (IFA; MP Biomedicals) at 9 and 10 weeks of age by subcutaneous injection at a dose of 20 μg/100 μl. Mice immunized with 100 μl IFA alone served as control. Mice were euthanized at 11 weeks of age. HLA-A*02:01 restricted antigen specific immune response was evaluated by flow cytometric analysis of splenocytes stained with CD19 (6D5), CD8a (KT15), and PE-conjugated HLA-A*02:01/A2V7-pentamer (ProImmune).

Atherosclerosis in A2Kb Tg ApoE^{-/-} Mice

A2Kb Tg ApoE^{-/-} mice were divided into two groups and fed normal chow or high cholesterol diet starting at 9 weeks of age until euthanasia at 17 or 25 weeks of age. RBC lysed splenocytes were stained for T effector/memory cell profile.

Another cohort of high cholesterol diet fed mice were euthanized at 17 weeks of age and the splenocytes stained with CD19 (6D5), CD8a (KT15), and PE-conjugated ApOB_{KTTKQSFDL} (SEQ ID NO:2) pentamer (ProImmune). A third cohort of female A2Kb Tg ApoE^{-/-} mice aged 66-68 weeks were fed high cholesterol diet for 4 weeks and euthanized to collect the whole aorta for enzymatic digestion with 0.25 mg/ml Collagenase, 0.125 mg/ml Elastase, and 60 U/ml Hyaluronidase (Sigma-Aldrich) in sterile RPMI 1640 medium for 20 minutes at 37° C. Single cell suspensions were then stained for ApOB_{KTTKQSFDL} (SEQ ID NO:2) pentamer and flow cytometric analysis as described above.

Immunization with P210-PAM in A2Kb Transgenic Mice

The first cohort of A2Kb Tg ApoE^{-/-} mice received either PBS or P210-PAM according to the same immunization protocol described prior for ApoE^{-/-} mice. Mice were sacrificed at 25 weeks of age and splenocytes were subject to flow cytometric analysis of ApOB_{KTTKQSFDL} (SEQ ID NO:2) pentamer (+) CD8+ T cells and their aorta for morphometric analysis of Oil-red-0 (+) plaques. To have a proper control for P210-PAM immunization, a second cohort of A2Kb Tg ApoE^{-/-} mice were immunized with MSA-PAM or P210-PAM using the same protocol and aorta analyzed for Oil-red-O (+) plaques.

Statistics

Data are presented as mean±SD. Number of animals in each group and statistical methods are listed in text, figures or figure legend. P<=0.05 was considered as statistically significant but trending data were also noted.

Preparation of Chimeric A2Kb Gene DNA Fragments for Fertilized Eggs Microinjection

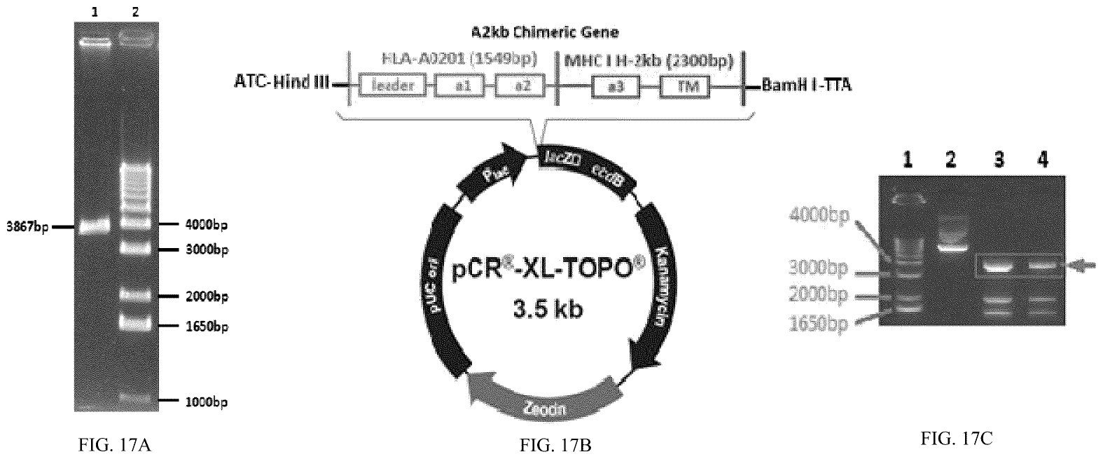

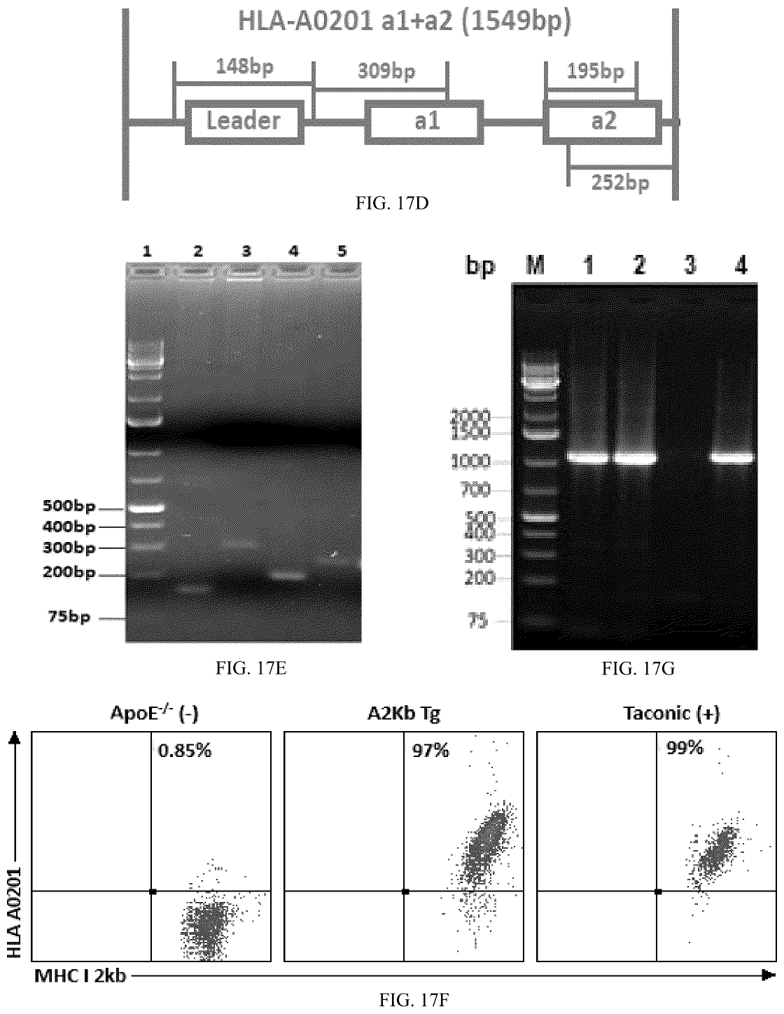

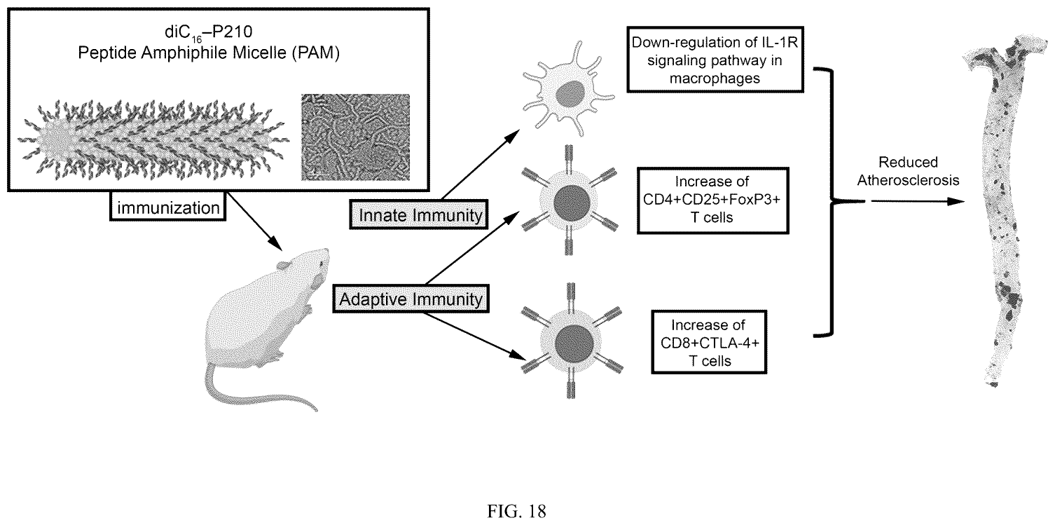

A 3867 bp full-length chimeric A2Kb gene containing sequence coding the leader sequence, α1 and α2 domains of HLA-A*02:01 and α3, transmembrane and cytoplasmic domains of the mouse MHC I H-2Kb gene (intron 3 to intron 8) was cloned by PCR using 35 cycles of 94° C. for 50 s, 56° C. for 50 s, and 68° C. for 4 min, with the genomic DNA from A2Kb transgenic CB6F1-Tg(HLA-A*02:01/H2-Kb)A*02:01 mouse as template (FIG. 17A). The following primers were used: F 5′-ATCAAGCTTACTCTCTGGCACCAAAC-3′ (SEQ ID NO:35), R 5′-TAAGGATCCCTAGTTGAGTCTCTGA-3′ (SEQ ID NO:36).

A2Kb PCR products purified with MONARCH® DNA Gel Extraction Kit (New England Biolabs, Cat #T1020S) were ligated with pCR-XL-TOPO T vectors (FIG. 17B) and transformed into competent E. coli cells following the kit's protocol (Thermo Fisher, Cat #K4750-10).

Recombinant plasmid clone with the right chimeric A2Kb sequence (sequenced by Laragen, Culver City, CA 90232) was amplified and purified by using Qiagen Plasmid Midi Kit (Cat #12143), following the protocol. Chimeric A2Kb fragments for microinjection were prepared by digesting 10-20 μg of the purified recombinant plasmids with restriction enzymes Hind III, BamH I and Hinc II (New England Biolabs, Cat #R0104S, R0136S, R0103S), at 37° C. for 4 hrs, followed by purification of the resulted A2Kb DNA fragments.

Generation of A2Kb Transgenic Founder by Fertilized Eggs Microinjection and Selection of A2Kb Transgenic Offspring for Experiments

Purified A2Kb fragments (˜3.9-kb, FIG. 17C) obtained as described were then microinjected into ApoE^{-/-} fertilized eggs by Rodent Genetics Core at Cedars-Sinai Medical Center. Germline-transmitted A2Kb chimeras obtained were screened by PCRs detecting 148 bp, 309 bp, 252 bp, 195 bp fragments coding part of leading peptide, α1, α2 domains of HLA-A*02:01 (FIGS. 17D & 17E) using toe genomic DNA (prepared by QuickExtract™ DNA Extraction Solution, Epicentre, Cat #QE09050) as template. Primers used for PCR are listed as following: HLA A*02:01 leader (148 bp): F 5′-ACTCAGATTCTCCCCAGACGC-3′ (SEQ ID NO:37) and R 5′-CCGTTGCTTCTCCCCACAGAG-3′ (SEQ ID NO:38); HLA A*0201 a1 (309 bp): F 5′-TGTGGGGAGAAGCAACGGG (SEQ ID NO:39) and R 5′-GAGTGGGCCTTCACTTTCCG (SEQ ID NO:40); HLA A*0201 a2 (195 bp): F 5′-GTTCTCACACCGTCCAGAGGAT-3′ (SEQ ID NO:41) and R 5′-ACTGCTCCGCCACATGGGCCGC-3′ (SEQ ID NO:42); HLA A*0201 a2 (252 bp): F 5′-TACCACCAGTACGCCTACGA-3′ (SEQ ID NO:43) and R 5′-ATCTACAGGCGATCAGGGAG-3′ (SEQ ID NO:44).

PCR results revealed one male chimera's genomic DNA might carry the A2Kb chimeric gene. A2Kb protein expression on the surface of peripheral blood mononuclear cells (PBMCs) in this mouse was further verified by flow cytometric analysis of cells stained with anti-human HLA-A2 (FITC, Clone: BB7.2, BD Bioscience, Cat #551285) and anti-mouse MHC-I H2Kb (PE, Clone AF6-88.5, BD Biosciences, Cat #553570). PBMCs from Taconic A2Kb Tg mice or ApoE^{-/-} mice were used as positive and negative control respectively (FIG. 17F).

The identified A2Kb transgenic ApoE^{-/-} male mouse then crossbred with female ApoE−/− mice, the A2Kb transgenic offspring used for further breeding and experiments were selected by flow cytometric analyzing the expression of chimeric A2Kb protein on surface of PBMCs. RT-PCR detecting a 1092 bp fragment of A2Kb mRNA (1113 bp of full-length) expression in splenic total RNAs of such A2Kb (+) offspring was further used to verify integration of the full-length A2Kb gene into the mouse genome (FIG. 17G), following a PCR program of 35 cycles of 940 C, 1 min; 620 C, 45 sec; 720 C, 1.5 min. Primers used for RT-PCR are F 5′-AACCCTCGTCCTGCTACTCT-3′ (SEQ ID NO:45) and R 5′-CACGCTAGAGAATGAGGGTCA-3′ (SEQ ID NO:46).

### Example 3. Immune Responses Against P210-PAM Vaccine in HLA-A*0201 Transgenic Mice

3.1 Immunization with P210-PAM in A2Kb-Tg ApoE^{-/-} Mice for Immune-Phenotyping

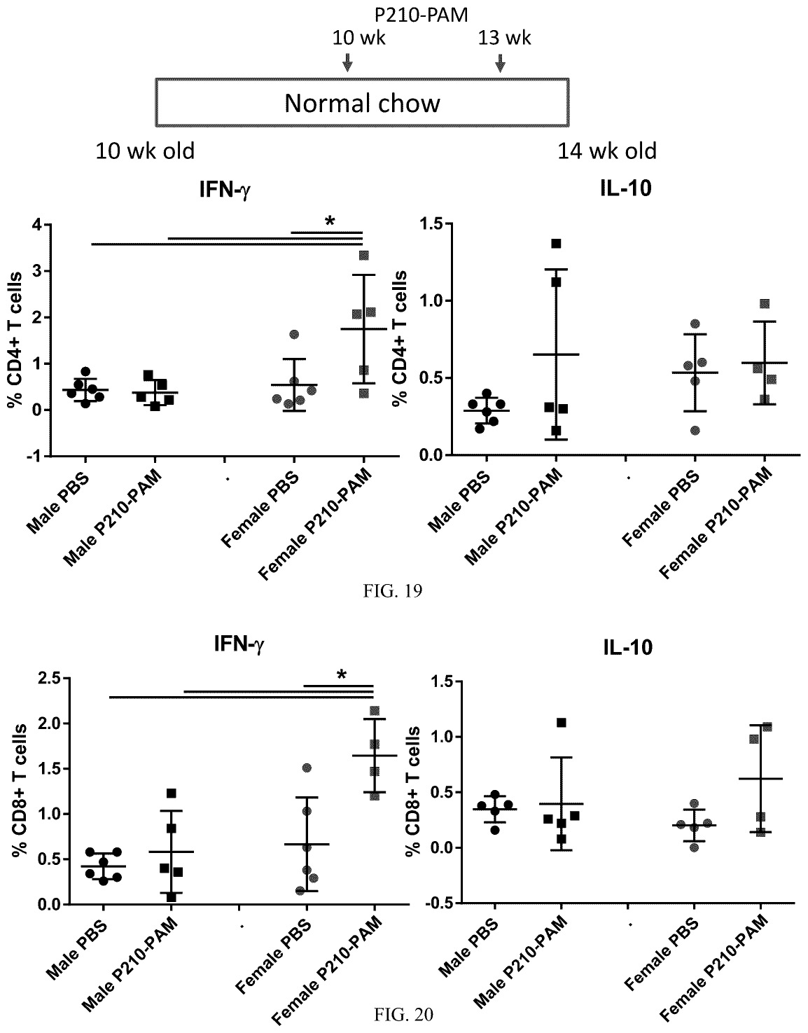

For the data presented in FIGS. 19-22, P210-PAM was administered twice to mice, at 10, 13 weeks old, respectively. Ten week-old male and female A2Kb-Tg ApoE^{-/-} mice fed normal chow received a subcutaneous injection of P210-PAM at the dose of 100 μg/100 ul/mouse. Booster injections were administered at 13 weeks of age. Mice were euthanized at 14 weeks of age, splenocytes were collected and t0 phenotype CD4⁺ and CD8⁺ T cells by flow cytometry.

FIG. 19 shows P210-PAM significantly increased CD4⁺ IFN-γ⁺ T cells in vaccinated female mice compared to control female and both groups of male mice. There was no difference in CD4⁺IL-10⁺ T cells.

FIG. 20 shows P210-PAM significantly increased CD8⁺IFN-γ⁺ T cells in vaccinated female mice compared to control female and both groups of male mice. There was no difference in CD8⁺IL-10⁺ T cells.

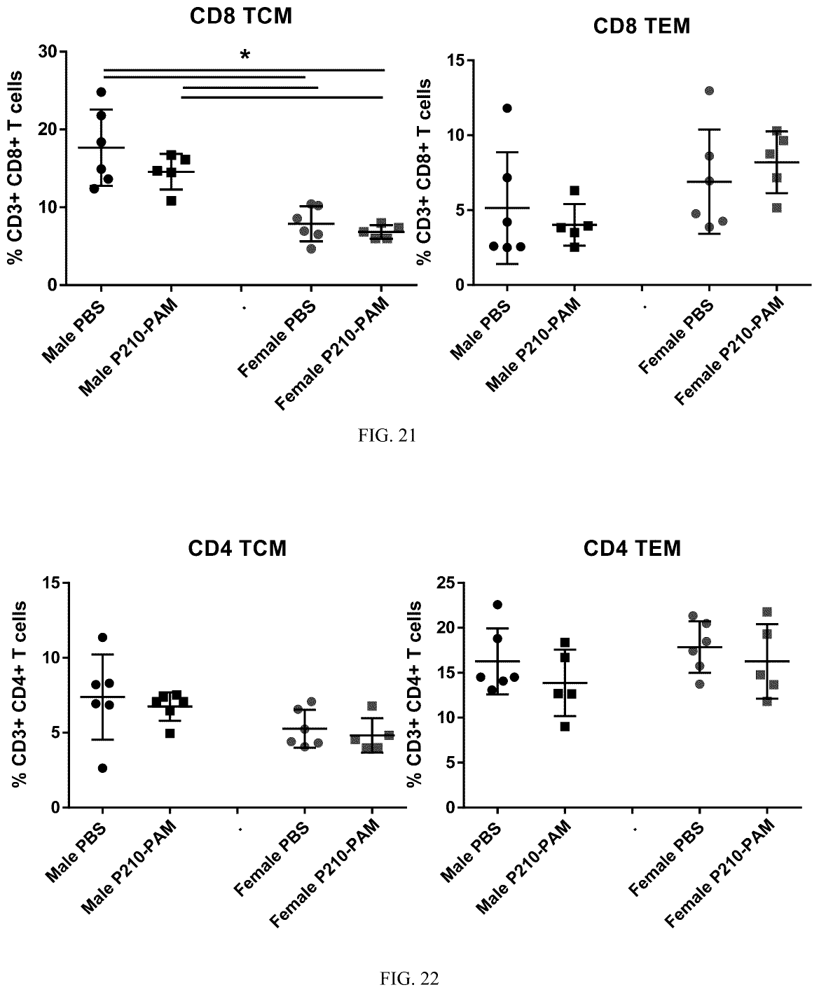

FIG. 21 shows P210-PAM significantly reduced CD8⁺ T_{CM} cells in female compared to male mice in both the control and P210-PAM vaccinated mice.

FIG. 22 shows there was no sex dependent difference in CD4⁺ T_{EM} and T_{CM} cells.

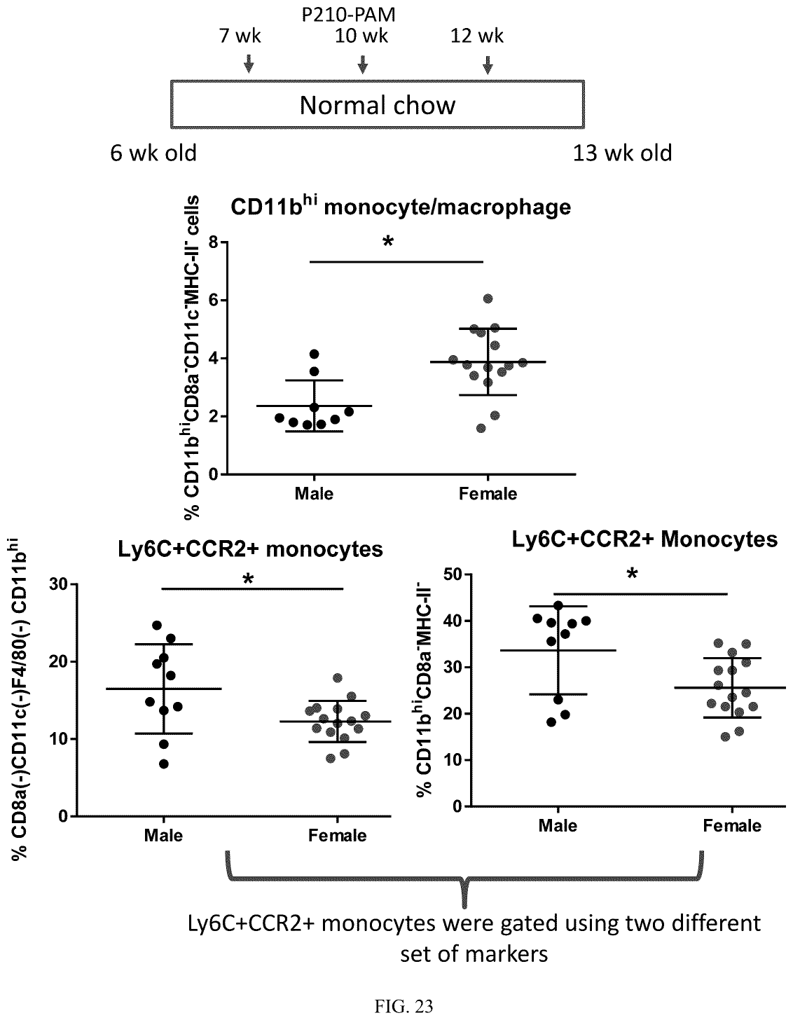

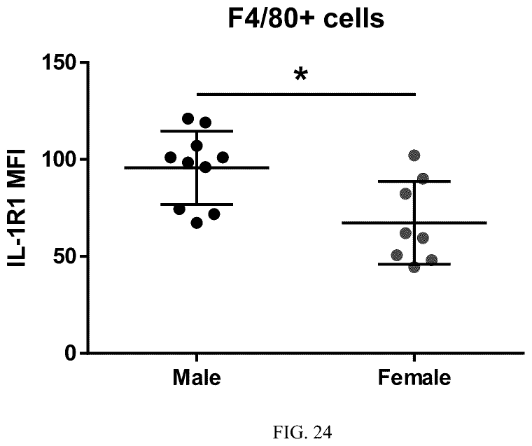

For FIGS. 23 and 24, P210-PAM were administered for three times, i.e., at 7, 10 and 13 weeks old. Seven-week-old male and female A2Kb-TgApoE^{-/-} mice fed with normal chow received a subcutaneous injection of P210-PAM at the dose of 100 μg/100 ul/mouse. Booster injections were administered at 10 and 12 weeks of age. Mice were euthanized at 13 weeks of age, splenocytes were collected and then stained with aqua fluorescent reactive dye and antibodies, to phenotype dendritic cells and monocytes/macrophages.

As seen in FIG. 23, P210-PAM significantly increased splenic CD11b^{hi} monocytes/macrophages in female compared to male mice, and P210-PAM significantly reduced Ly6C⁺CCR2⁺ monocytes in female compared to male mice.

FIG. 24 shows P210-PAM significantly reduced surface IL-1R1 expression on splenic F4/80+ macrophages in female compared to male mice.

Hence, P210-PAM immunization elicited preferential IFN-gamma response in female mice, indicating sex-dependent response to vaccine. The effect of significantly reduced splenic CD8⁺ T_{CM} cells in female mice with or without immunization when compared to male mice was not seen in CD4 T cells. Preferential changes on monocyte/macrophages in female mice indicates sex-dependent innate immune responses. Although this mouse experiment appeared to favor female mice, we conceive that P210-PAM may have effects on human subjects, both male and female.

3.2 Immunization with P210-PAM in A2Kb-Tg ApoE^{-/-} Mice on Different High Cholesterol Diet Feeding Protocols: Athero-Protection by P210-PAM Vaccine in Mice and its Interaction with Cholesterol-Lowering.

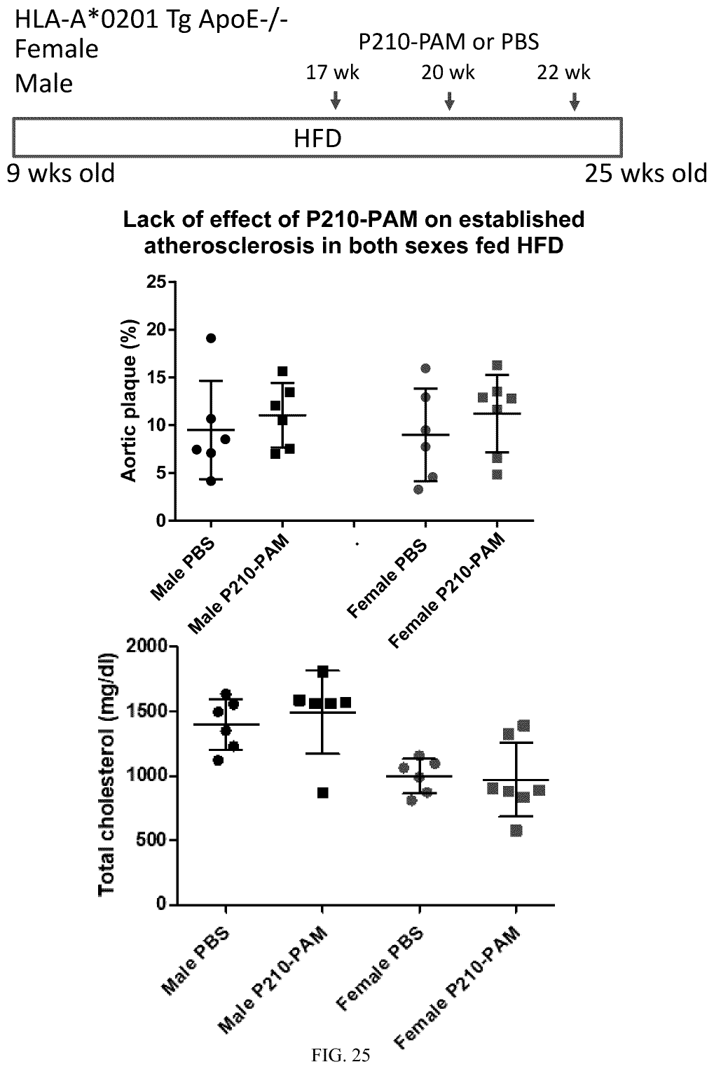

Protocol 1 (exemplified in FIG. 25): A2Kb-Tg ApoE^{-/-} mice were fed high cholesterol diet (Envigo, TD.88137) starting at 9 weeks of age until their euthanization. Each group of mice received 3 times subcutaneous injection of P210-PAM (100 μg/100 ul/mouse) at 17, 20 and 22 weeks of age; and mice receiving PBS served as control. Mice were euthanized at 25 weeks of age, whole aortas were cleaned, processed and stained with Oil-red-0 to assess the extent of atherosclerosis en face.

FIG. 25 shows P210-PAM immunization did not lead to any significant difference in established atherosclerosis, quantified by aortic plaque percentage, compared to PBS, in either A2Kb-Tg ApoE^{-/-} male mice or A2Kb-Tg ApoE^{-/-} female mice, when mice were fed with high fat diet throughout a period from at least 8 weeks prior to first dose of P210-PAM until at least 3 weeks after the last dose of P210-PAM.

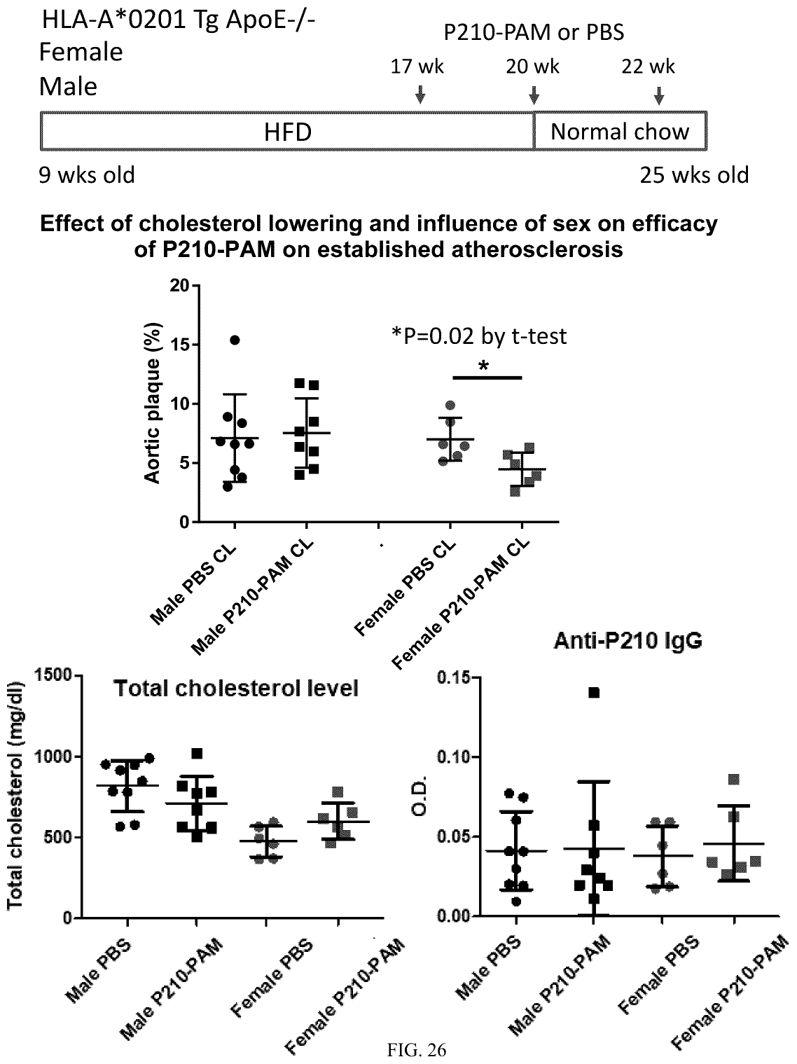

Protocol 2 (exemplified in FIG. 26): to investigate the effect of cholesterol lowering by diet on P210-PAM immunized mice, separate groups of A2Kb-Tg ApoE^{-/-} mice immunized with P210-PAM or PBS with the same immunization protocol were fed on high cholesterol diet from 9 weeks to 20 weeks of age and then switched to normal chow until euthanization at 25 weeks of age.

FIG. 26 shows the effect of cholesterol lowering by diet and the influence of sex on the efficacy of P210-PAM on established atherosclerosis. P210-PAM immunization significantly reduced established atherosclerosis, quantified by aortic plaque percentage, compared to PBS, in female A2Kb-Tg ApoE^{-/-} mice with a lowered cholesterol intake (e.g., fed with normal chow in place of high fat diet) for a period beginning from at least the first booster shot of P210-PAM through at least the second booster shot.

Hence, cholesterol lowering in conjunction with P210-PAM immunization reduced established atherosclerosis only in female mice. This indicated sex dependent immune functions in atherosclerosis in the context of immune modulation with an ApoB-100 antigen in mice. Although this mouse experiment appeared to favor female mice, we conceive that P210-PAM may have effects on human subjects, both male and female.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A peptide-amphiphile complex, comprising a lipophilic portion covalently bonded to a peptide portion at the amino-terminal end of the peptide portion, wherein the peptide portion comprises a sequence of KTTKQSFDLSVKAQYKKNKH (SEQ ID NO:1) or a fragment of SEQ ID NO:1 capable of binding a human leukocyte antigen (HLA).

2. The peptide-amphiphile complex of claim 1, wherein the lipophilic portion comprises two linear alkyl chains.

3. The peptide-amphiphile complex of claim 2, wherein each linear alkyl chain has 6 to 20 carbon atoms.

4. A peptide-amphiphile complex, comprising a lipophilic portion and a peptide portion, the peptide-amphiphile complex having the following structure: 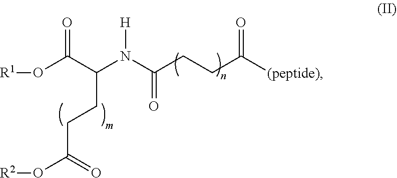
or a variant of (II), wherein the variant has any one or more of —O— or ═O in (II) be independently substituted with another atom than oxygen;
wherein R¹ and R² are each independently C₆-C₂₀ substituted or unsubstituted hydrocarbyl groups; m and n are independently a positive integer or 0, representing the number or absence of repeats of unsubstituted or substituted —CH₂—CH₂—; and the (peptide) refers to a sequence of KTTKQSFDLSVKAQYKKNKH (SEQ ID NO:1) or a fragment of SEQ ID NO:1 capable of binding a human leukocyte antigen (HLA).

5. The peptide-amphiphile complex of claim 4, wherein R¹ and R² are independently C₁₂-C₁₆ hydrocarbyl groups, and the (peptide) refers to the sequence of SEQ ID NO:1.

6. The peptide-amphiphile complex of claim 1, further comprising a detectable label, optionally a fluorescent label.

7. The peptide-amphiphile complex of claim 1, which is in the form of a micelle or a vesicle in a pharmaceutically acceptable medium.

8. A pharmaceutical composition, comprising (a) nanoparticles each comprising a quantity of the peptide-amphiphile complex of claim 1, and (b) a pharmaceutically acceptable excipient.

9. The pharmaceutical composition of claim 8, wherein the nanoparticle is a micellar nanofiber formed from the quantity of the peptide-amphiphile complex, and the peptide-amphiphile complex has a structure of: 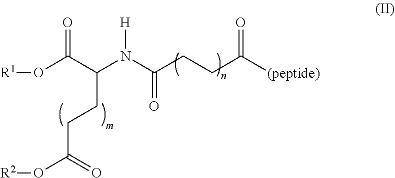
wherein R¹ and R² are each independently C₁₂-C₁₆ hydrocarbyl groups; m and n are independently a positive integer or 0, representing the number or absence of repeats of unsubstituted or substituted —CH₂—CH₂—; and the (peptide) comprises the sequence of SEQ ID NO:1.

10. The pharmaceutical composition of claim 8, wherein the pharmaceutically acceptable excipient comprises one or more pharmaceutically innocuous fillers and/or adjuvants.

11. An immunogenic composition for eliciting an immune response in a mammal having an ischemic cardiovascular disease, comprising the pharmaceutical composition of claim 8, wherein the nanoparticles comprise an immunogenically effective amount of the peptide-amphiphile complex, and the pharmaceutical composition optionally further comprises an adjuvant.

12. The immunogenic composition of claim 11, wherein the nanoparticles are micelles; and wherein the pharmaceutical composition does not include a major histocompatibility complex (MHC) molecule.

13. A method for eliciting an immune response or providing a therapeutic treatment in a subject having atherosclerosis or an ischemic cardiovascular disease, comprising administering to the subject a therapeutically effective amount of the pharmaceutical composition of claim 8.

14. The method of claim 13, wherein the therapeutically effective amount reduces cytolytic activity of CD8⁺ T cell, reduces proliferative activity of CD4⁺ T cell, reduces aortic atherosclerosis, or a combination thereof, in the subject.

15. The method of claim 13, for eliciting a therapeutic treatment by reducing amount of plaques in the cardiovasculature of the subject, wherein the subject has acute coronary syndrome or an atherosclerotic cardiovascular disease before the administration.

16. The method of claim 15, wherein the subject is a human.

17. The method of claim 13, for eliciting a protective immune response, wherein the subject does not have acute coronary syndrome or atherosclerotic cardiovascular disease.

18. The method of claim 13, wherein the pharmaceutical composition is administered in a series comprising a first dose and one or more booster doses.

19. A method of making a micellar composition composed of the peptide-amphiphile complex of claim 1, comprising the steps of:
(a) drying a liquid film comprising the peptide-amphiphile complex having been dissolved in an organic solvent, to result in a lipid film comprising the peptide-amphiphile complex;
(b) hydrating the lipid film comprising the peptide-amphiphile complex in an aqueous medium, wherein the aqueous medium is heated to a temperature above a gel-liquid crystal transition temperature of the peptide-amphiphile complex, thereby obtaining a hydrated lipid suspension comprising the peptide-amphiphile complex; and
(c) subjecting the hydrated lipid suspension comprising the peptide-amphiphile complex to sonication or extrusion, so as to obtain a micellar composition composed of the peptide-amphiphile complex.

20. The method of claim 19, wherein the peptide-amphiphile complex has a structure of: 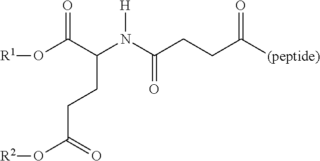
wherein each R¹ and R² are independently C₁₂-C₁₆ hydrocarbyl groups, and the (peptide) comprises a contiguous sequence of SEQ ID NO:1; and
wherein the micellar composition is a cylindrical nanofiber comprising the peptide-amphiphile complex.


SEQUENCE LISTING (47 sequences)

SEQ ID NO: 1 - protein, 20 aa, Homo sapiens
KTTKQSFDLSVKAQYKKNKH

SEQ ID NO: 2 - protein, 9 aa, Homo sapiens
KTTKQSFDL

SEQ ID NO: 3 - protein, 9 aa, Homo sapiens
TTKQSFDLS

SEQ ID NO: 4 - protein, 9 aa, Homo sapiens
TKQSFDLSV

SEQ ID NO: 5 - protein, 9 aa, Homo sapiens
KQSFDLSVK

SEQ ID NO: 6 - protein, 9 aa, Homo sapiens
QSFDLSVKA

SEQ ID NO: 7 - protein, 9 aa, Homo sapiens
SFDLSVKAQ

SEQ ID NO: 8 - protein, 9 aa, Homo sapiens
FDLSVKAQY

SEQ ID NO: 9 - protein, 9 aa, Homo sapiens
DLSVKAQYK

SEQ ID NO: 10 - protein, 9 aa, Homo sapiens
LSVKAQYKK

SEQ ID NO: 11 - protein, 9 aa, Homo sapiens
SVKAQYKKN

SEQ ID NO: 12 - protein, 9 aa, Homo sapiens
VKAQYKKNK

SEQ ID NO: 13 - protein, 9 aa, Homo sapiens
KAQYKKNKH

SEQ ID NO: 14 - DNA, 20 bp, synthetic construct
  source 1..20: mol_type = other DNA
atcactgccacccagaagac

SEQ ID NO: 15 - DNA, 20 bp, synthetic construct
  source 1..20: mol_type = other DNA
cacattgggggtaggaacac

SEQ ID NO: 16 - DNA, 20 bp, synthetic construct
  source 1..20: mol_type = other DNA
ggcagaggtccagaagaatg

SEQ ID NO: 17 - DNA, 20 bp, synthetic construct
  source 1..20: mol_type = other DNA
gccagagatgcttggaactg

SEQ ID NO: 18 - DNA, 20 bp, synthetic construct
  source 1..20: mol_type = other DNA
agtggtccaacctgcaggtc

SEQ ID NO: 19 - DNA, 20 bp, synthetic construct
  source 1..20: mol_type = other DNA
ctgatgttgccattgttggt

SEQ ID NO: 20 - DNA, 20 bp, synthetic construct
  source 1..20: mol_type = other DNA
cagccagatgcagttaacgc

SEQ ID NO: 21 - DNA, 23 bp, synthetic construct
  source 1..23: mol_type = other DNA
gcctactcattgggatcatcttg

SEQ ID NO: 22 - DNA, 21 bp, synthetic construct
  source 1..21: mol_type = other DNA
ctgcaagagacttccatccag

SEQ ID NO: 23 - DNA, 23 bp, synthetic construct
  source 1..23: mol_type = other DNA
agtggtatagacaggtctgttgg

SEQ ID NO: 24 - DNA, 21 bp, synthetic construct
  source 1..21: mol_type = other DNA
tttgaattccctgggtgagaa

SEQ ID NO: 25 - DNA, 21 bp, synthetic construct
  source 1..21: mol_type = other DNA
acaggggagaaatcgatgaca

SEQ ID NO: 26 - DNA, 20 bp, synthetic construct
  source 1..20: mol_type = other DNA
cacgctacctcctctttttg

SEQ ID NO: 27 - DNA, 21 bp, synthetic construct
  source 1..21: mol_type = other DNA
cagcagtgcaggaataatgtt

SEQ ID NO: 28 - DNA, 20 bp, synthetic construct
  source 1..20: mol_type = other DNA
gggcctcaaaggaaagaatc

SEQ ID NO: 29 - DNA, 20 bp, synthetic construct
  source 1..20: mol_type = other DNA
ttgcttgggatccacactct

SEQ ID NO: 30 - DNA, 20 bp, synthetic construct
  source 1..20: mol_type = other DNA
caggagaagtcgcaggaagt

SEQ ID NO: 31 - DNA, 20 bp, synthetic construct
  source 1..20: mol_type = other DNA
tggaacagagccagtgtcag

SEQ ID NO: 32 - DNA, 20 bp, synthetic construct
  source 1..20: mol_type = other DNA
tctctgatgctgttgctgct

SEQ ID NO: 33 - DNA, 20 bp, synthetic construct
  source 1..20: mol_type = other DNA
cgtggaacggttgaggtagt

SEQ ID NO: 34 - protein, 9 aa, synthetic construct
VLSDFKTWL

SEQ ID NO: 35 - DNA, 26 bp, synthetic construct
  source 1..26: mol_type = other DNA
atcaagcttactctctggcaccaaac

SEQ ID NO: 36 - DNA, 25 bp, synthetic construct
  source 1..25: mol_type = other DNA
taaggatccctagttgagtctctga

SEQ ID NO: 37 - DNA, 21 bp, synthetic construct
  source 1..21: mol_type = other DNA
actcagattctccccagacgc

SEQ ID NO: 38 - DNA, 21 bp, synthetic construct
  source 1..21: mol_type = other DNA
ccgttgcttctccccacagag

SEQ ID NO: 39 - DNA, 19 bp, synthetic construct
  source 1..19: mol_type = other DNA
tgtggggagaagcaacggg

SEQ ID NO: 40 - DNA, 20 bp, synthetic construct
  source 1..20: mol_type = other DNA
gagtgggccttcactttccg

SEQ ID NO: 41 - DNA, 22 bp, synthetic construct
  source 1..22: mol_type = other DNA
gttctcacaccgtccagaggat

SEQ ID NO: 42 - DNA, 22 bp, synthetic construct
  source 1..22: mol_type = other DNA
actgctccgccacatgggccgc

SEQ ID NO: 43 - DNA, 20 bp, synthetic construct
  source 1..20: mol_type = other DNA
taccaccagtacgcctacga

SEQ ID NO: 44 - DNA, 20 bp, synthetic construct
  source 1..20: mol_type = other DNA
atctacaggcgatcagggag

SEQ ID NO: 45 - DNA, 20 bp, synthetic construct
  source 1..20: mol_type = other DNA
aaccctcgtcctgctactct

SEQ ID NO: 46 - DNA, 21 bp, synthetic construct
  source 1..21: mol_type = other DNA
cacgctagagaatgagggtca

SEQ ID NO: 47 - protein, 20 aa, Mus musculus
QTALAELVKHKPKATAEQLK